(19) United States
(12) Patent Application Publication
(10) Pub. No.: US 2020/0075866 A1
(43) Pub. Date: Mar. 5, 2020

(54) COMPOSITION FOR ORGANIC OPTOELECTRONIC DEVICE AND ORGANIC OPTOELECTRONIC DEVICE AND DISPLAY DEVICE

(71) Applicants: SAMSUNG SDI CO., LTD., Yongin-si (KR); SAMSUNG ELECTRONICS CO., LTD., Suwon-si (KR)

(72) Inventors: Byoungkwan LEE, Suwon-si (KR); Dong Min KANG, Suwon-si (KR); Jun Seok KIM, Suwon-si (KR); Bang-Lin LEE, Suwon-si (KR); Sangshin LEE, Suwon-si (KR); Eun Sun YU, Suwon-si (KR); Namheon LEE, Suwon-si (KR); Ho Kuk JUNG, Suwon-si (KR); Youngkyoung JO, Suwon-si (KR); Bowon CHOI, Suwon-si (KR)

(21) Appl. No.: 16/558,550

(22) Filed: Sep. 3, 2019

(30) Foreign Application Priority Data
Sep. 4, 2018 (KR) 10-2018-0105491

(51) Int. Cl. H01L 51/00; C07D 307/77; C09K 11/06; C07D 405/12; C07D 307/91; C07D 409/14; C07D 333/50; C07D 251/24; C07D 405/04

(52) U.S. Cl. CPC H01L 51/0061; H01L 51/5016; C09K 11/06; H01L 51/006; C07D 405/12; C07D 307/91; C07D 409/14; C07D 333/50; C07D 251/24; H01L 51/0067; H01L 51/0054; C07D 405/04; H01L 51/0073; H01L 51/0058; C07D 307/77

(57) ABSTRACT

A composition, an organic optoelectronic device, and a display device, the composition including a first compound represented by a combination of Chemical Formula 1 and Chemical Formula 2, and a second compound represented by Chemical Formula 3:

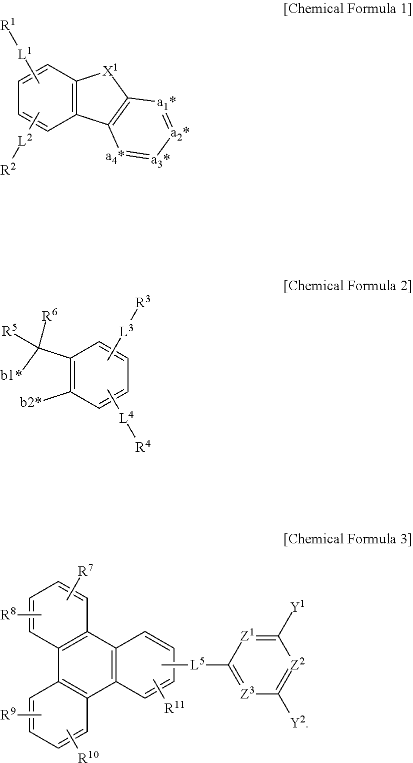

### CROSS-REFERENCE TO RELATED APPLICATION

Korean Patent Application No. 10-2018-0105491, filed on Sep. 4, 2018, in the Korean Intellectual Property Office, and entitled: “Composition for Organic Optoelectronic Device and Organic Optoelectronic Device and Display Device,” is incorporated by reference herein in its entirety.

### BACKGROUND

### 1. Field

Embodiments relate to a composition for an organic optoelectronic device, an organic optoelectronic device, and a display device are disclosed.

### 2. Description of the Related Art

An organic optoelectronic device is a device that converts electrical energy into photoenergy, and vice versa.

An organic optoelectronic device may be classified as follows in accordance with its driving principles. One is a photoelectric device where excitons generated by photoenergy are separated into electrons and holes and the electrons and holes are transferred to different electrodes respectively and electrical energy is generated, and the other is a light emitting device to generate photoenergy from electrical energy by supplying a voltage or a current to electrodes.

Examples of the organic optoelectronic device may include an organic photoelectric device, an organic light emitting diode, an organic solar cell, and an organic photo conductor drum.

Among them, the organic light emitting diode (OLED) has recently drawn attention due to an increase in demand for flat panel displays. The organic light emitting diode may convert electrical energy into light, and the performance of organic light emitting diode may be influenced by the organic materials between electrodes.

### SUMMARY

The embodiments may be realized by providing a composition for an organic optoelectronic device, the composition including a first compound represented by a combination of Chemical Formula 1 and Chemical Formula 2, and a second compound represented by Chemical Formula 3:

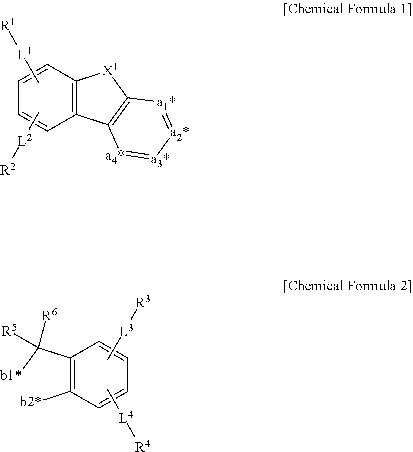

wherein, in Chemical Formula 1 and Chemical Formula 2, X¹ is O or S, adjacent two of a₁* to a₄* are C linked with b₁* and b₂* respectively, the other two of a₁* to a₄* not linked with b₁* and b₂* are each independently C-L^{a}-R^{a}, L^{a} and L¹ to L⁴ are each independently a single bond, a substituted or unsubstituted C6 to C20 arylene group, a substituted or unsubstituted C2 to C20 heterocyclic group, or a combination thereof, R^{a} and R¹ to R⁶ are each independently hydrogen, deuterium, a cyano group, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, and at least one of R¹ to R⁴ is a substituted amine group represented by Chemical Formula a,

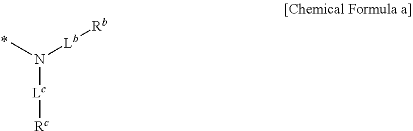

wherein, in Chemical Formula a, L^{b} and L^{c} are each independently a single bond, a substituted or unsubstituted C6 to C20 arylene group, a substituted or unsubstituted C2 to C20 heterocyclic group, or a combination thereof, R^{b} and R^{c} are each independently a substituted or unsubstituted C6 to C30 aryl group, a substituted or unsubstituted C2 to C30 heterocyclic group, or a combination thereof, and * is a linking point with L¹, L², L³, or L⁴;

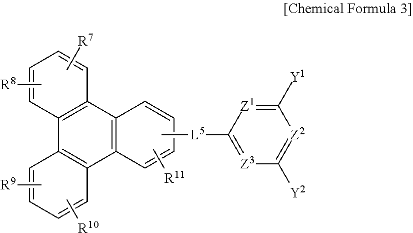

wherein, in Chemical Formula 3, Z¹ to Z³ are each independently N or CR^{d}, at least two of Z¹ to Z³ are N, Y¹ and Y² are each independently hydrogen, deuterium, a substituted or unsubstituted C1 to C30 alkyl group, a substituted or unsubstituted C6 to C30 aryl group, a substituted or unsubstituted C2 to C30 heterocyclic group, a halogen, a cyano group, or a combination thereof, L⁵ is a single bond or a substituted or unsubstituted C6 to C20 arylene group, and R⁷ to R¹¹ and R^{d} are each independently hydrogen, deuterium, a substituted or unsubstituted C1 to C30 alkyl group, a substituted or unsubstituted C6 to C30 aryl group, a halogen, a cyano group, or a combination thereof.

The first compound may be represented by one of Chemical Formula 1A to Chemical Formula 1F:

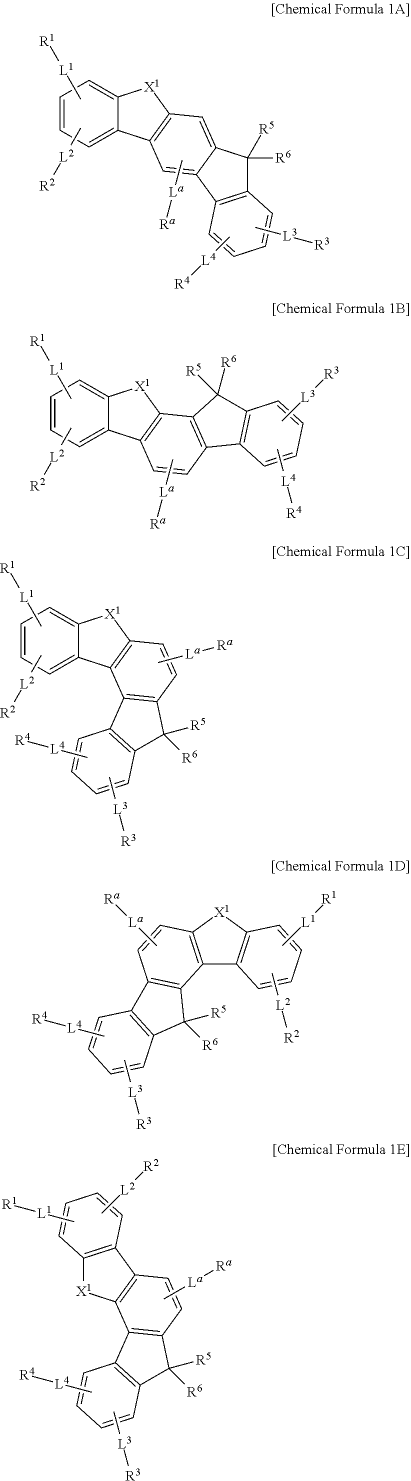

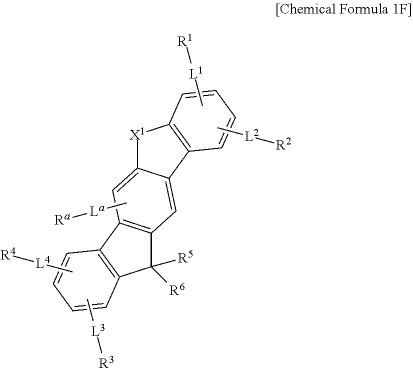

wherein, in Chemical Formula 1A to Chemical Formula 1F, X¹ is O or S, L^{a} and L¹ to L⁴ are each independently a single bond, a substituted or unsubstituted C6 to C20 arylene group, a substituted or unsubstituted C2 to C20 heterocyclic group, or a combination thereof, R^{a} and R¹ to R⁶ are each independently hydrogen, deuterium, a cyano group, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, and at least one of R¹ to R⁴ is a substituted amine group represented by Chemical Formula a,

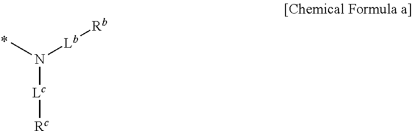

wherein, in Chemical Formula a, L^{b} and L^{c} are each independently a single bond, a substituted or unsubstituted C6 to C20 arylene group, a substituted or unsubstituted C2 to C20 heterocyclic group, or a combination thereof, R^{b} and R^{c} are each independently a substituted or unsubstituted C6 to C30 aryl group, a substituted or unsubstituted C2 to C30 heterocyclic group, or a combination thereof, and * is a linking point with L¹, L², L³, or L⁴.

The first compound may be represented by Chemical Formula 1E-1-1 or Chemical Formula 1E-2-2:

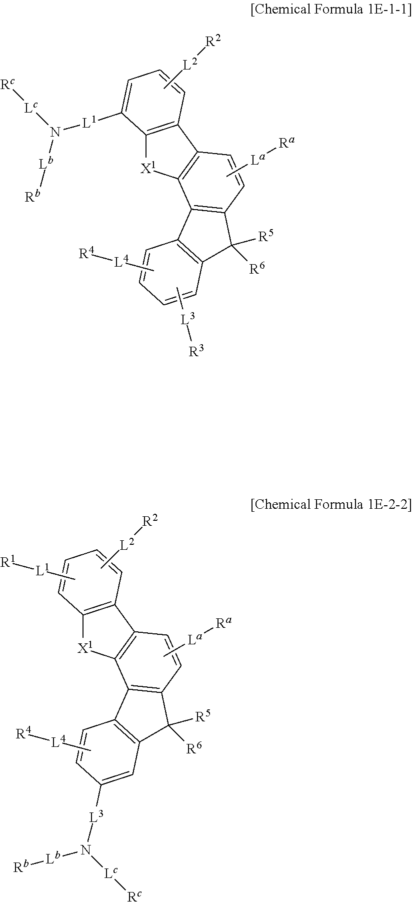

wherein, in Chemical Formula 1E-1-1 and Chemical Formula 1E-2-2, X¹ is O or S, L^{a} and L¹ to L⁴ are each independently a single bond, a substituted or unsubstituted C6 to C20 arylene group, a substituted or unsubstituted C2 to C20 heterocyclic group, or a combination thereof, R^{a} and R¹ to R⁶ are each independently hydrogen, deuterium, a cyano group, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, L^{b} and L^{c} are each independently a single bond, a substituted or unsubstituted C6 to C20 arylene group, a substituted or unsubstituted C2 to C20 heterocyclic group, or a combination thereof, and R^{b} and R^{c} are each independently a substituted or unsubstituted phenyl group, a substituted or unsubstituted biphenyl group, a substituted or unsubstituted terphenyl group, a substituted or unsubstituted anthracenyl group, a substituted or unsubstituted naphthyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted dibenzofuranyl group, a substituted or unsubstituted dibenzothiophenyl group, or a monovalent fused ring group of a compound represented by a combination of Chemical Formula 1 and Chemical Formula 2.

The second compound may be represented by Chemical Formula 3A or Chemical Formula 3B:

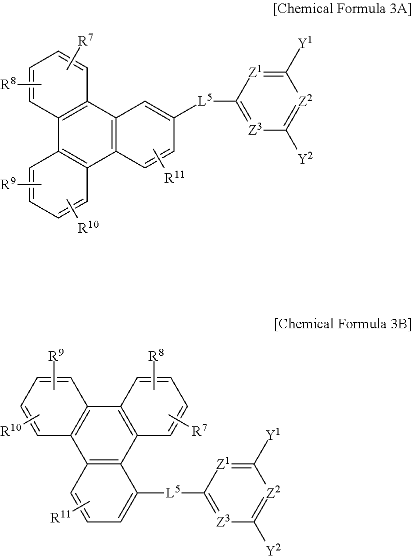

wherein, in Chemical Formulae 3A and 3B, Z¹ to Z³ are each independently N or CR^{d}, at least two of Z¹ to Z³ are N, Y¹ and Y² are each independently hydrogen, deuterium, a substituted or unsubstituted C1 to C30 alkyl group, a substituted or unsubstituted C6 to C30 aryl group, a substituted or unsubstituted C2 to C30 heterocyclic group, a halogen, a cyano group, or a combination thereof, L⁵ is a single bond or a substituted or unsubstituted C6 to C20 arylene group, and R⁷ to R¹¹ and R^{d} are each independently hydrogen, deuterium, a substituted or unsubstituted C1 to C30 alkyl group, a substituted or unsubstituted C6 to C30 aryl group, a halogen, a cyano group, or a combination thereof.

Y¹ and Y² may each independently be a substituted or unsubstituted phenyl group, a substituted or unsubstituted biphenyl group, a substituted or unsubstituted terphenyl group, a substituted or unsubstituted naphthyl group, a substituted or unsubstituted triphenylene group, a substituted or unsubstituted pyridinyl group, a substituted or unsubstituted pyrimidinyl group, a substituted or unsubstituted triazinyl group, a substituted or unsubstituted dibenzofuranyl group, a substituted or unsubstituted dibenzothiophenyl group, or a combination thereof.

L⁵ may be a single bond, a substituted or unsubstituted phenylene group, a substituted or unsubstituted biphenylene group, or a substituted or unsubstituted terphenylene group.

L⁵ may be a single bond, an unsubstituted phenylene group, an unsubstituted biphenylene group, an unsubstituted terphenylene group, a phenylene group substituted with a phenyl group or a cyano group, a biphenylene group substituted with a phenyl group or a cyano group, or a terphenylene group substituted with a phenyl group or a cyano group.

L⁵ may be a single bond or a linking group of the following Group I,

### [Group I]

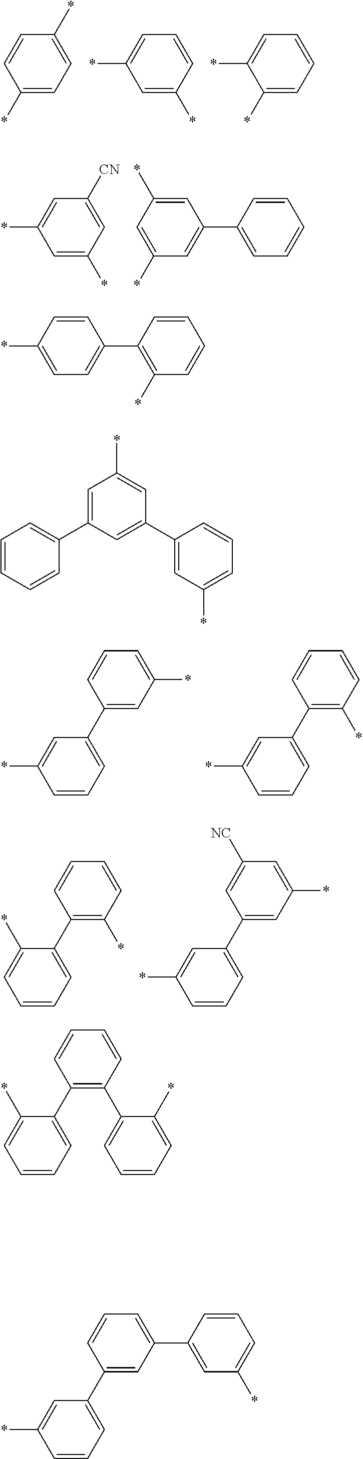

wherein, in Group I, each * is a linking point.

The moiety

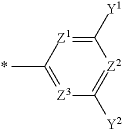

of Chemical Formula 3 may be a moiety of the following Group II,

### [Group II]

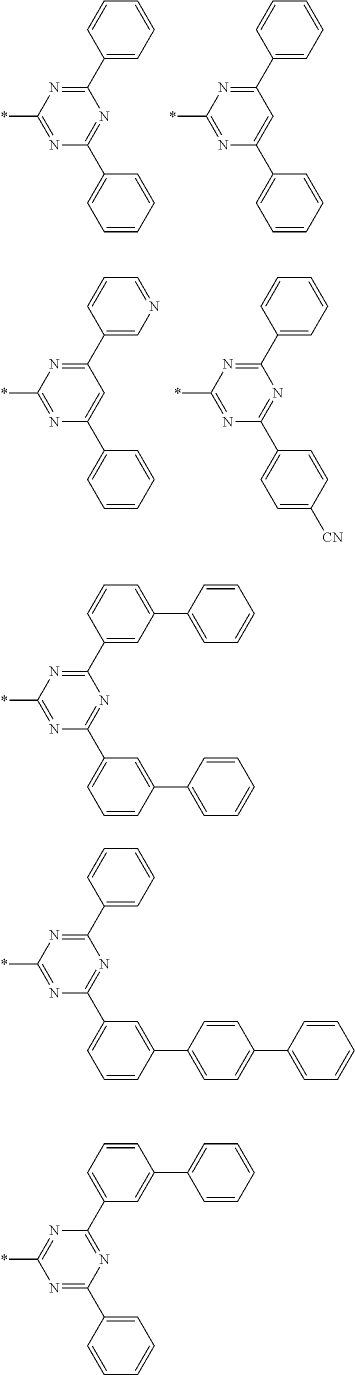

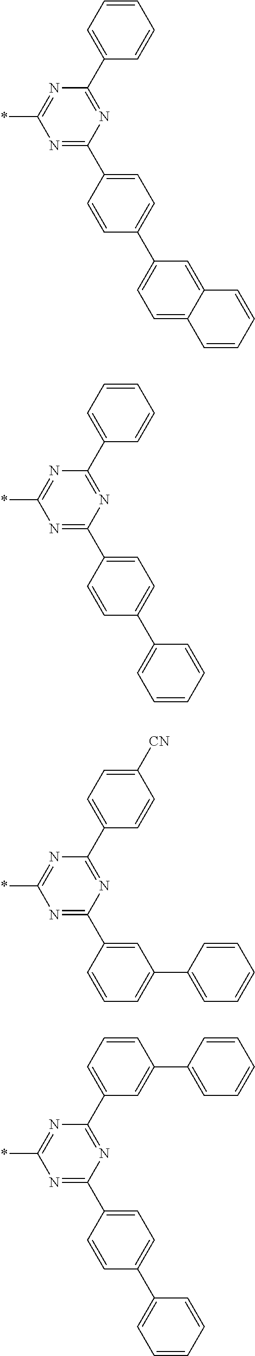

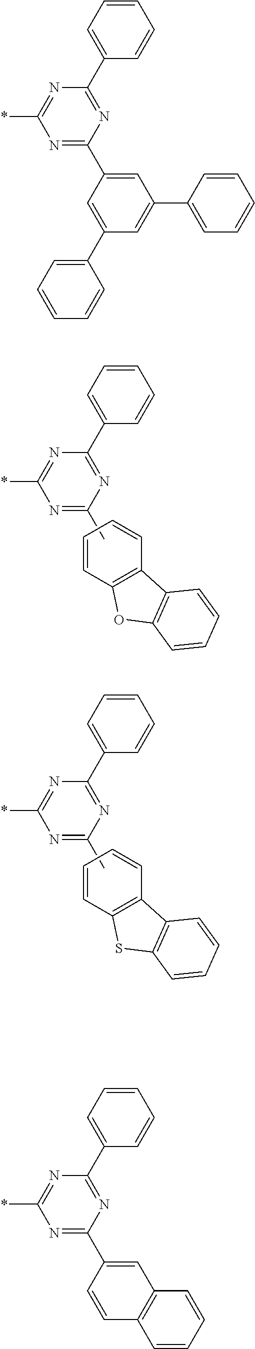

wherein, in Group II, * is a linking point.

The first compound may be represented by Chemical Formula 1E-2-2, and the second compound may be represented by Chemical Formula 3A-1:

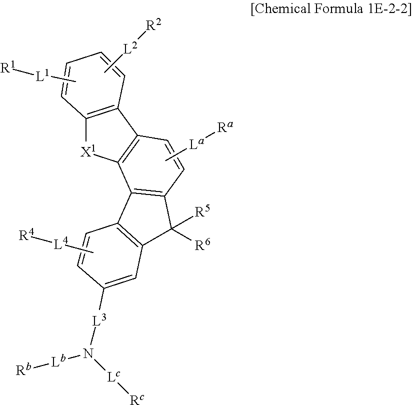

wherein, in Chemical Formula 1E-2-2, X¹ is O or S, L^{a}, L^{b}, L^{c}, and L¹ to L⁴ are each independently a single bond, a substituted or unsubstituted phenylene group, a substituted or unsubstituted biphenylene group, a substituted or unsubstituted terphenylene group, or a substituted or unsubstituted naphthylene group, R^{a}, R¹, R², and R⁴ are each independently hydrogen, deuterium, a cyano group, a substituted or unsubstituted C1 to C30 alkyl group, a substituted or unsubstituted C6 to C30 aryl group, a substituted or unsubstituted C2 to C30 heterocyclic group, or a combination thereof, R⁵ and R⁶ are each independently a substituted or unsubstituted C1 to C10 alkyl group or a substituted or unsubstituted C6 to C20 aryl group, and R^{b} and R^{c} are each independently a substituted or unsubstituted phenyl group, a substituted or unsubstituted biphenyl group, a substituted or unsubstituted anthracenyl group, a substituted or unsubstituted naphthyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted dibenzofuranyl group, a substituted or unsubstituted dibenzothiophenyl group, or a monovalent fused ring group of a compound represented by a combination of Chemical Formula 1 and Chemical Formula 2;

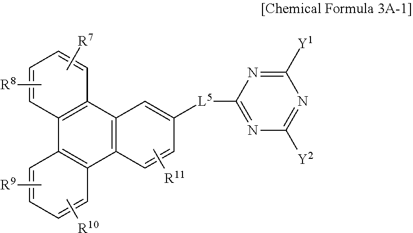

wherein, in Chemical Formula 3A-1, Y¹ and Y² are each independently hydrogen, deuterium, a substituted or unsubstituted C1 to C30 alkyl group, a substituted or unsubstituted C6 to C30 aryl group, a substituted or unsubstituted C2 to C30 heterocyclic group, a halogen, a cyano group, or a combination thereof, L⁵ is a single bond or a substituted or unsubstituted C6 to C20 arylene group, and R⁷ to R¹¹ are each independently hydrogen, deuterium, a substituted or unsubstituted C1 to C30 alkyl group, a substituted or unsubstituted C6 to C30 aryl group, a halogen, a cyano group, or a combination thereof.

The composition may further include a dopant.

The embodiments may be realized by providing an organic optoelectronic device including an anode and a cathode facing each other, at least one organic layer between the anode and the cathode, wherein the organic layer includes the composition for an organic optoelectronic device according to an embodiment.

The organic layer may include a light emitting layer, and the light emitting layer may include the composition.

The first compound and the second compound may be phosphorescent hosts of the light emitting layer.

The composition may be a red light emitting composition.

The embodiments may be realized by providing a display device including the organic optoelectronic device according to an embodiment.

### BRIEF DESCRIPTION OF THE DRAWINGS

Features will become apparent to those of skill in the art by describing in detail exemplary embodiments with reference to the attached drawings in which:

FIGS. 1 and 2 illustrate cross-sectional views showing organic light emitting diodes according to embodiments.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figures, the dimensions of layers and regions may be exaggerated for clarity of illustration. It will also be understood that when a layer or element is referred to as being “on” another layer or element, it can be directly on the other layer or element, or intervening layers may also be present. In addition, it will also be understood that when a layer is referred to as being “between” two layers, it can be the only layer between the two layers, or one or more intervening layers may also be present. Like reference numerals refer to like elements throughout.

As used herein, when a definition is not otherwise provided, “substituted” may refer to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In an implementation, “substituted” may refer to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, or a C2 to C30 heteroaryl group. In addition, in specific examples of the present invention, “substituted” refers to replacement of at least one hydrogen of a substituent or a compound by deuterium, a C1 to C20 alkyl group, a C6 to C30 aryl group, or a C2 to C30 heteroaryl group. In addition, in specific examples of the present invention, “substituted” refers to replacement of at least one hydrogen of a substituent or a compound by deuterium, a C1 to C5 alkyl group, a C6 to C18 aryl group, a pyridinyl group, a quinolinyl group, an isoquinolinyl group, a dibenzofuranyl group, a dibenzothiophenyl group, or a carbazolyl group. In addition, in specific examples of the present invention, “substituted” refers to replacement of at least one hydrogen of a substituent or a compound by deuterium, a C1 to C5 alkyl group, a C6 to C18 aryl group, a dibenzofuranyl group, or a dibenzothiophenyl group. In addition, in specific examples of the present invention, “substituted” refers to replacement of at least one hydrogen of a substituent or a compound by deuterium, a methyl group, an ethyl group, a propanyl group, a butyl group, a phenyl group, a biphenyl group, a terphenyl group, a naphthyl group, a triphenyl group, a dibenzofuranyl group, or a dibenzothiophenyl group.

As used herein, when a definition is not otherwise provided, “hetero” may refer to one including one to three heteroatoms selected from N, O, S, P, and Si, and remaining carbons in one functional group.

In the present specification, “aryl group” may refer to a group including at least one hydrocarbon aromatic moiety, and may include a group in which all elements of the hydrocarbon aromatic moiety have p-orbitals which form conjugation, for example a phenyl group, a naphthyl group, and the like, a group in which two or more hydrocarbon aromatic moieties may be linked by a sigma bond, for example a biphenyl group, a terphenyl group, a quarterphenyl group, and the like, and a group in which two or more hydrocarbon aromatic moieties are fused directly or indirectly to provide a non-aromatic fused ring, for example a fluorenyl group, and the like.

The aryl group may include a monocyclic, polycyclic or fused ring polycyclic (i.e., rings sharing adjacent pairs of carbon atoms) functional group.

In the present specification, “heterocyclic group” is a generic concept of a heteroaryl group, and may include at least one heteroatom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” may refer to an aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

For example, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, or a combination thereof.

For example, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzothiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted dibenzofuranyl group, or a substituted or unsubstituted dibenzothiophenyl group, or a combination thereof.

In the present specification, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a composition for an organic optoelectronic device according to an embodiment is described.

The composition for an organic optoelectronic device may include, e.g., a first compound (for an organic optoelectronic device) having hole characteristics and a second compound (for an organic optoelectronic device) having electron characteristics.

The first compound may be represented by a combination of Chemical Formula 1 and Chemical Formula 2.

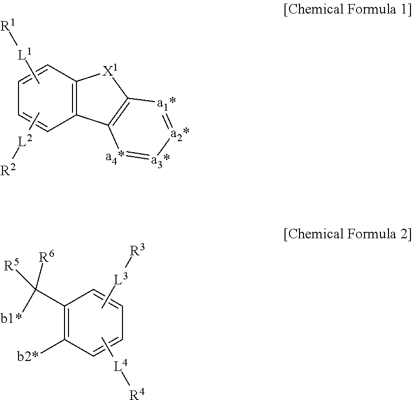

In Chemical Formula 1 and Chemical Formula 2,

X¹ may be, e.g., O or S,

adjacent two of a₁* to a₄* may be C linked with b₁* and b₂* respectively,

the remainders (e.g., the other two of) of a₁* to a₄* not linked with b₁* and b₂* may each independently be, e.g., C-L^{a}-R^{a},

L^{a} and L¹ to L⁴ may each independently be or include, e.g., a single bond, a substituted or unsubstituted C6 to C20 arylene group, a substituted or unsubstituted C2 to C20 heterocyclic group, or a combination thereof, and

R^{a} and R¹ to R⁶ may each independently be or include, e.g., hydrogen, deuterium, a cyano group, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof. For example, remaining carbon atoms of the rings that include -L¹-R¹, -L²-R², -L³-R³, and -L⁴-R⁴ thereon may have a hydrogen atom bonded thereto.

In an implementation, at least one of R¹ to R⁴ may be, e.g., a (substituted amine) group represented by Chemical Formula a,

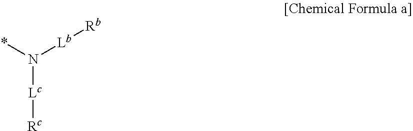

In Chemical Formula a,

L^{b} and L^{c} may each independently be or include, e.g., a single bond, a substituted or unsubstituted C6 to C20 arylene group, a substituted or unsubstituted C2 to C20 heterocyclic group, or a combination thereof.

R^{b} and R^{c} may each independently be or include, e.g., a substituted or unsubstituted C6 to C30 aryl group, a substituted or unsubstituted C2 to C30 heterocyclic group, or a combination thereof (e.g., a monovalent group of a fused ring represented by a combination of Chemical Formula 1 and Chemical Formula 2).

* is a linking point with L¹ to L⁴.

The first compound may have a structure of an amine (substituted with an aryl group and/or a heteroaryl group) linked to a fused heterocycle of 6 membered ring-5 membered ring-6 membered ring-5 membered ring-6 membered ring and may have high HOMO energy. For example, a HOMO electron cloud may expand from the amine to the fused heterocycle, and the compound may exhibit excellent hole injection and transfer characteristics.

In addition, the fused heterocycle of 6 membered ring-5 membered ring-6 membered ring-5 membered ring-6 membered ring may have relatively high HOMO energy compared with bicarbazole and indolocarbazole, and a device having a low driving voltage may be realized by applying the structure of the amine linked to the fused heterocycle.

In addition, bicarbazole and indolocarbazole may have high T1 energy and may not be appropriate or suitable as a red host. In contrast, the structure of the amine linked to the fused heterocycle (e.g., according to an embodiment) may have appropriate T1 energy as a red host.

The first compound may include the fused heterocycle and may exhibit a decreased symmetry in the molecule, and crystallization among the compounds may be suppressed. For example, a dark spot generated due to the crystallization of the compounds during deposition of a material in a process of manufacturing a device may be suppressed, and accordingly, a life-span of the device may be improved.

For example, a device manufactured with the first compound according to an embodiment may realize high efficiency/long life-span characteristics.

In an implementation, the first compound may be included with the second compound, may exhibit satisfactory interface characteristics and transportation capability of holes and electrons and accordingly, and may lower a driving voltage of a device manufactured by applying the same.

In an implementation, L^{b} and L^{c} may each independently be, e.g., a single bond or a substituted or unsubstituted C6 to C12 arylene group.

In an implementation, L^{b} and L^{c} may each independently be, e.g., a single bond, a substituted or unsubstituted phenylene group, or a substituted or unsubstituted biphenylene group.

In an implementation, R^{b} and R^{c} may each independently be or include, e.g., a substituted or unsubstituted phenyl group, a substituted or unsubstituted biphenyl group, a substituted or unsubstituted terphenyl group, a substituted or unsubstituted anthracenyl group, a substituted or unsubstituted naphthyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted dibenzofuranyl group, a substituted or unsubstituted dibenzothiophenyl group, or a monovalent group of a fused ring represented by a combination of Chemical Formula 1 and Chemical Formula 2. For example, R^{b} and/or R^{c} may be a monovalent group that is a fused combination of a substituted or unsubstituted C6 to C30 aryl group and a substituted or unsubstituted C2 to C30 heterocyclic group that forms a monovalent fused ring group of the compound represented by a combination of Chemical Formula 1 and Chemical Formula 2. For example, R^{b} and/or R^{c} may be a monovalent group of a fused heterocycle of 6 membered ring-5 membered ring-6 membered ring-5 membered ring-6 membered ring. For example, a hydrogen of the combination of Chemical Formula 1 and Chemical Formula 2 may be replaced with a bonding location to L^{b}, L^{c}, and/or N in order to form a monovalent group for R^{b} and/or R^{c}.

In an implementation, R^{b} and R^{c} may each independently be or include, e.g., a substituted or unsubstituted phenyl group, a substituted or unsubstituted biphenyl group, a substituted or unsubstituted naphthyl group, a substituted or unsubstituted fluorenyl group, a substituted or unsubstituted carbazolyl group, a substituted or unsubstituted dibenzofuranyl group, a substituted or unsubstituted dibenzothiophenyl group, or a fused ring represented by a combination of Chemical Formula 1 and Chemical Formula 2.

In an implementation, R^{b} and R^{c} may each independently be or include, e.g., a substituted or unsubstituted phenyl group, a substituted or unsubstituted biphenyl group, a substituted or unsubstituted naphthyl group, or a substituted or unsubstituted fluorenyl group.

In an implementation, L^{a} and L¹ to L⁴ may each independently be or include, e.g., a single bond or a substituted or unsubstituted C6 to C20 arylene group.

In an implementation, L^{a} and L¹ to L⁴ may each independently be or include, e.g., a single bond, a substituted or unsubstituted phenylene group, a substituted or unsubstituted biphenylene group, or a substituted or unsubstituted naphthylene group.

In an implementation, L^{a} and L¹ to L⁴ may each independently be or include, e.g., a single bond or a substituted or unsubstituted p-phenylene group.

In an implementation, R^{a} and R¹ to R⁴ may each independently be or include, e.g., hydrogen, deuterium, a cyano group, a substituted or unsubstituted C1 to C10 alkyl group, or a substituted or unsubstituted C6 to C20 aryl group.

In an implementation, R^{a} and R¹ to R⁴ may each be, e.g., hydrogen.

In an implementation, R⁵ and R⁶ may each independently be or include, e.g., a substituted or unsubstituted C1 to C10 alkyl group or a substituted or unsubstituted C6 to C20 aryl group.

In an implementation, R⁵ and R⁶ may each independently be or include, e.g., a substituted or unsubstituted C1 to C4 alkyl group or a substituted or unsubstituted C6 to C12 aryl group.

In an implementation, the first compound may be a compound represented by one of Chemical Formula 1A to Chemical Formula 1F (e.g., according to a combination point of Chemical Formula 1 and Chemical Formula 2).

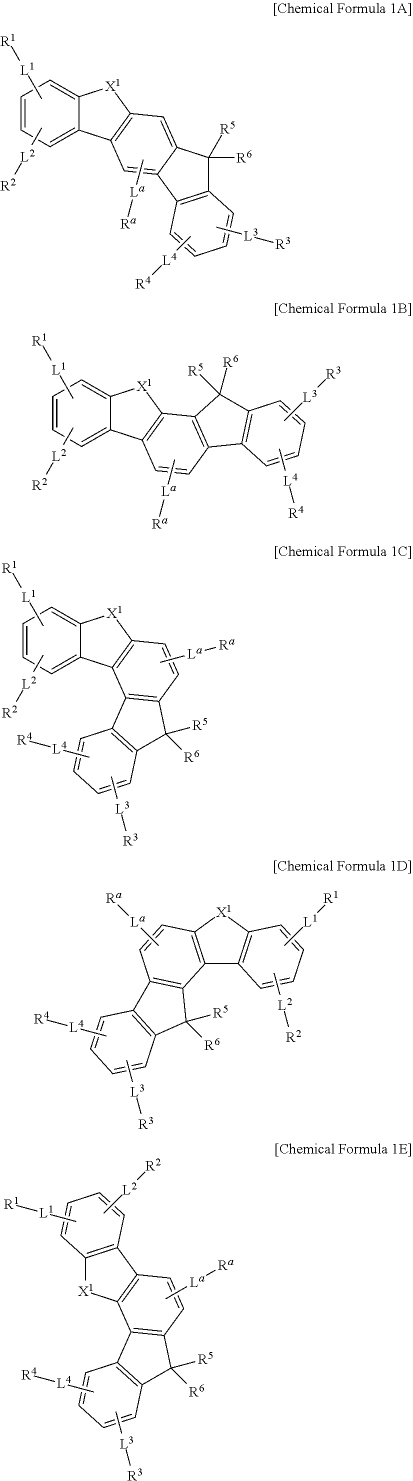

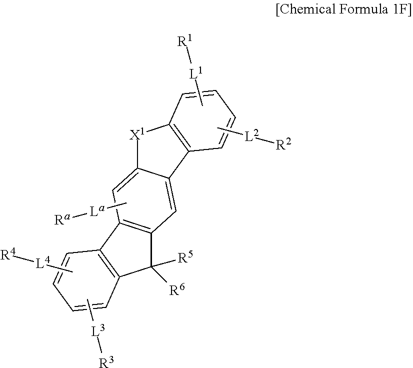

In Chemical Formula 1A to Chemical Formula 1F, X¹, L^{a} and L¹ to L⁴ and R^{a} and R¹ to R⁶ may be defined the same as those of Chemical Formulae 1 and 2.

In an implementation, the first compound represented by Chemical Formula 1A may be represented by Chemical Formula 1A-1 or Chemical Formula 1A-2 (e.g., according to a substitution direction of the group represented by Chemical Formula a).

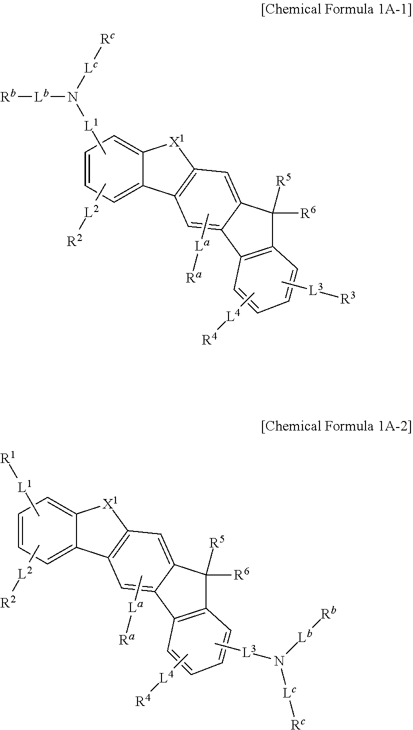

In Chemical Formula 1A-1 and Chemical Formula 1A-2, X¹, L^{a}, L^{b}, L^{c}, L¹ to L⁴, R^{a}, R¹ to R⁶, R^{b}, and R^{c} may be defined the same as those of Chemical Formulae 1 and 2.

In an implementation, the first compound represented by Chemical Formula 1A-1 may be represented by one of Chemical Formula 1A-1-1 to Chemical Formula 1A-1-4 (e.g., according to a specific substitution position of the group represented by Chemical Formula a).

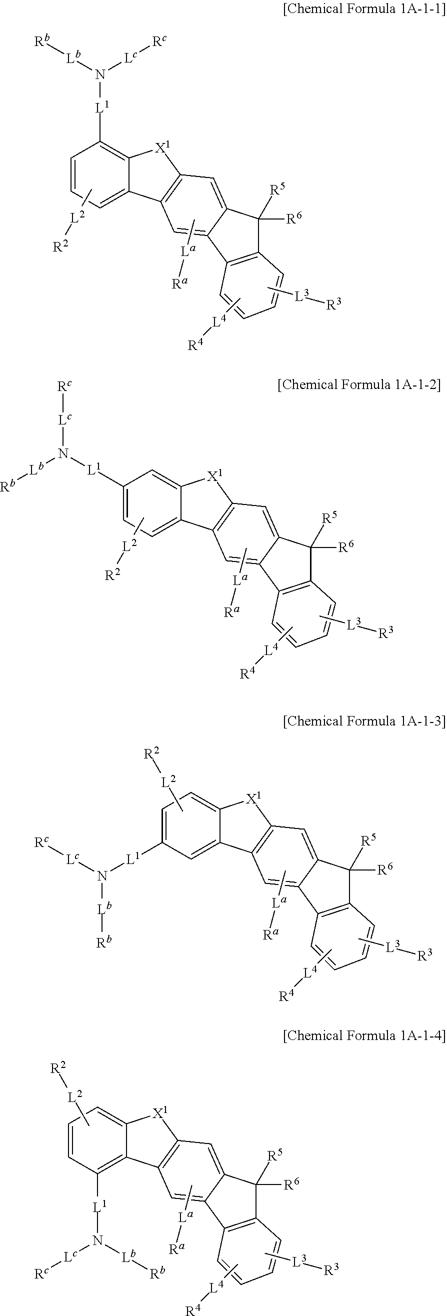

In Chemical Formula 1A-A-1-1 to Chemical Formula A-1-4, X¹, L^{a}, L^{b}, L^{c}, L¹ to L⁴, R^{a}, R¹ to R⁶, R^{b}, and R^{c} may be defined the same as those of Chemical Formulae 1 and 2.

In an implementation, the first compound represented by Chemical Formula 1A-2 may be represented by one of Chemical Formula 1A-2-1 to Chemical Formula 1A-2-4 (e.g., according to a specific substitution position of the group represented by Chemical Formula a).

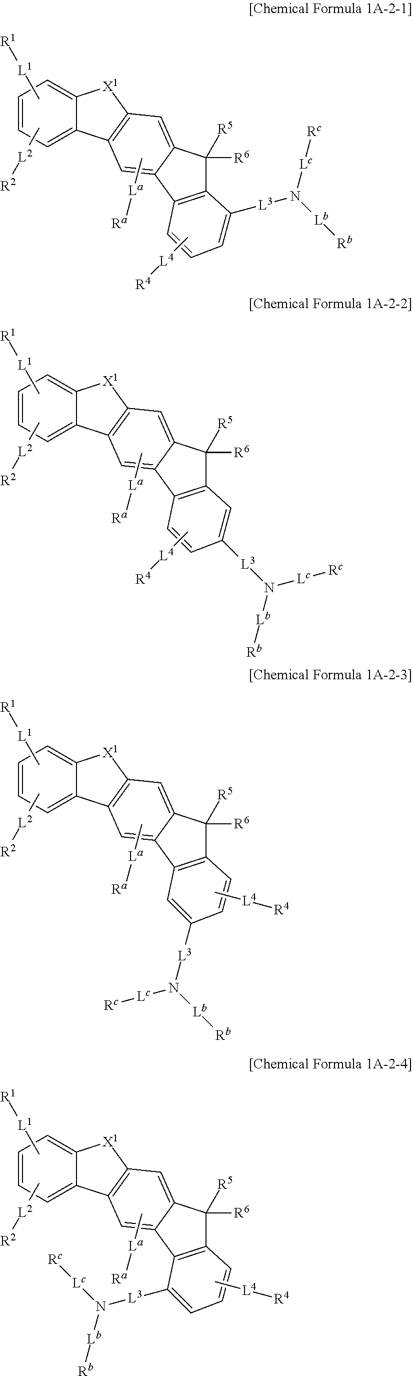

In Chemical Formula 1A-2-1 to Chemical Formula 1A-2-4, X¹, L^{a}, L^{b}, L^{c} and L¹ to L⁴ and R¹ to R⁶ and R^{b} and R^{c} may be defined the same as those of Chemical Formulae 1 and 2.

In an implementation, the first compound represented by Chemical Formula 1A may be represented by one of Chemical Formula 1A-1-1, Chemical Formula 1A-2-2, and Chemical Formula 1A-2-3.

In an implementation, the first compound represented by Chemical Formula 1B may be represented by Chemical Formula 1B-1 or Chemical Formula 1B-2 (e.g., according to a substitution direction of the group represented by Chemical Formula a).

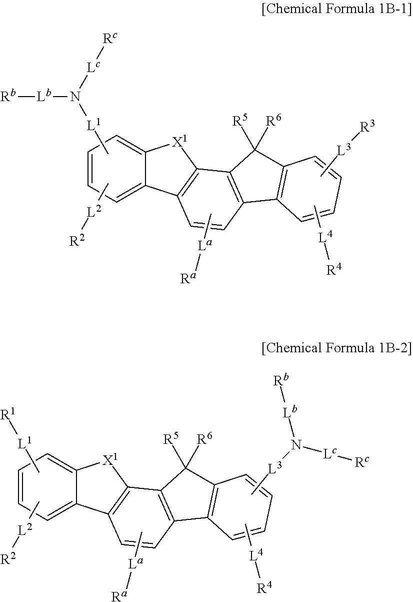

In Chemical Formula 1B-1 and Chemical Formula 1B-2, X¹, L^{a}, L^{b}, L^{c}, L¹ to L⁴, R^{a}, R¹ to R⁶, R^{b}, and R^{c} may be defined the same as those of Chemical Formulae 1 and 2.

In an implementation, the first compound represented by Chemical Formula 1B-1 may be represented by one of Chemical Formula 1B-1-1 to Chemical Formula 1B-1-4 (e.g., according to a specific substitution position of the group represented by Chemical Formula a).

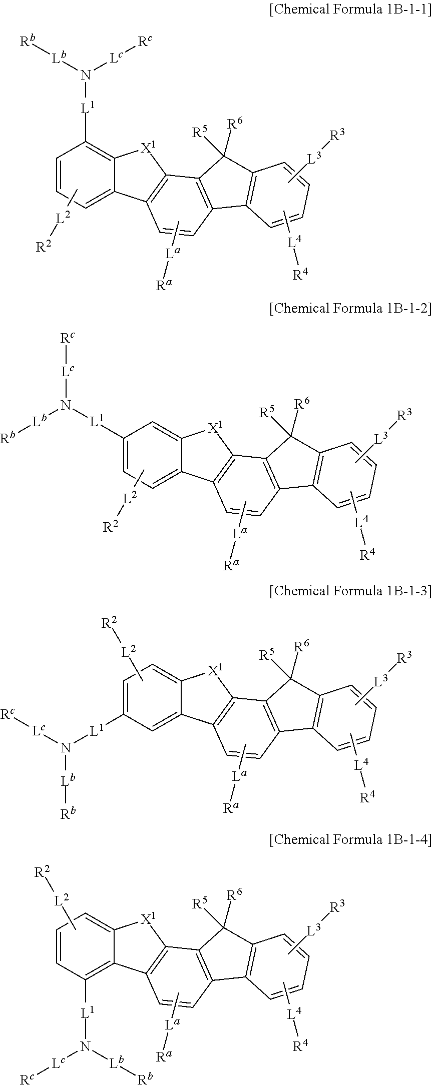

In Chemical Formula 1B-1-1 to Chemical Formula 1B-1-4, X¹, L^{a}, L^{b}, L^{c}, L¹ to L⁴, R^{a}, R¹ to R⁶, R^{b}, and R may be defined the same as those of Chemical Formulae 1 and 2.

In an implementation, the first compound represented by Chemical Formula 1B-2 may be represented by one of Chemical Formula 1B-2-1 to Chemical Formula 1B-2-4 (e.g., according to a substitution position of the group represented by Chemical Formula a).

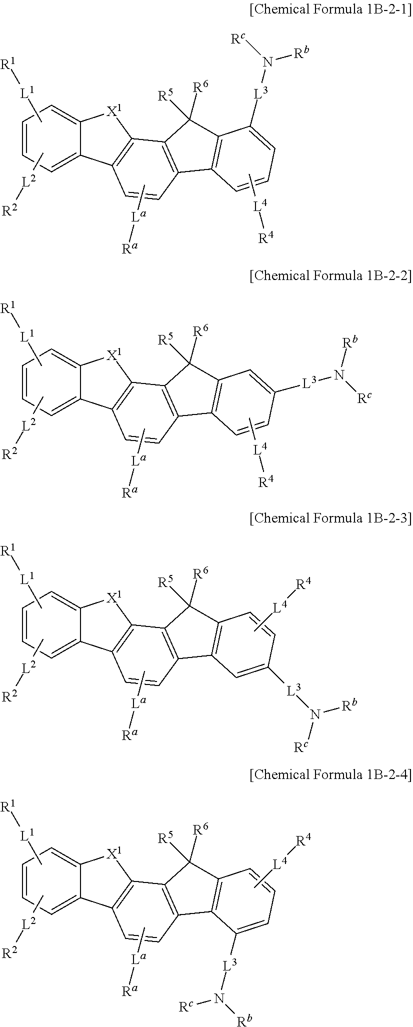

In Chemical Formula 1B-2-1 to Chemical Formula 1B-2-4, X¹, L^{a}, L^{b}, L^{c}, L¹ to L⁴, R^{a}, R¹ to R⁶, R^{b}, and R^{c} may be defined the same as those of Chemical Formulae 1 and 2.

In an implementation, the first compound represented by Chemical Formula 1B may be represented by one of Chemical Formula 1B-1-1, Chemical Formula 1B-2-2, and Chemical Formula 1B-2-3.

In an implementation, the first compound represented by Chemical Formula 1C may be represented by Chemical Formula 1C-1 or Chemical Formula 1C-2 (e.g., according to a substitution direction of the group represented by Chemical Formula a).

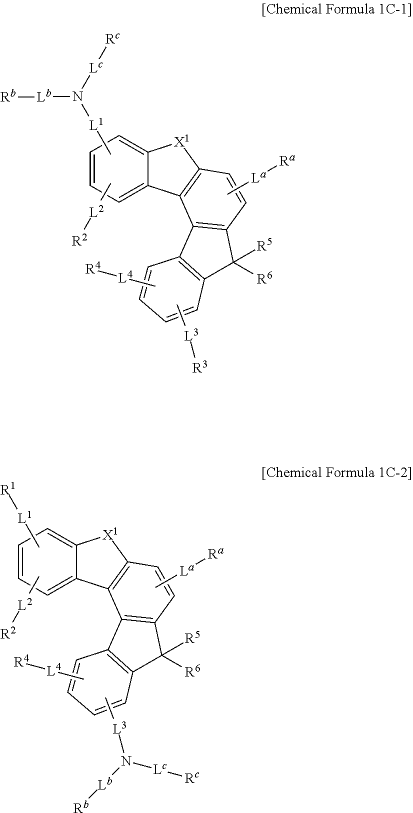

In Chemical Formula 1C-1 and Chemical Formula 1C-2, X¹, L^{a}, L^{b}, L^{c}, L¹ to L⁴, R^{a}, R¹ to R⁶, R^{b}, and R^{c} may be defined the same as those of Chemical Formulae 1 and 2.

In an implementation, the first compound represented by Chemical Formula 1C-1 may be represented by one of Chemical Formula 1C-1-1 to Chemical Formula 1C-1-4 (e.g., according to a specific substitution position of the group represented by Chemical Formula a).

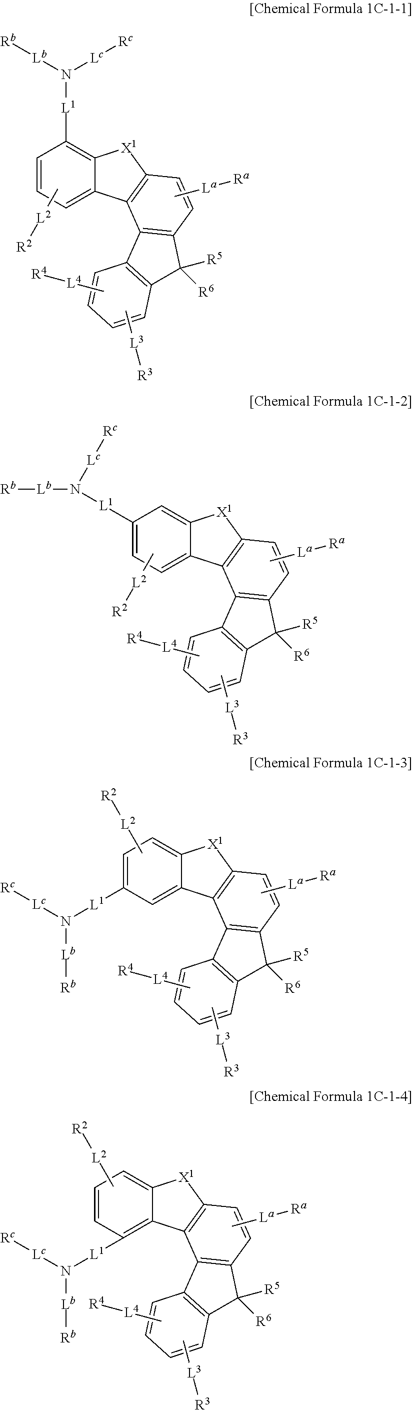

In Chemical Formula 1C-1-1 to Chemical Formula 1C-1-4, X¹, L¹, L^{b}, L^{c}, L¹ to L⁴, R^{a}, R¹ to R⁶, R^{b}, and R^{c} may be defined the same as those of Chemical Formulae 1 and 2.

In an implementation, the first compound represented by Chemical Formula 1C-2 may be represented by one of Chemical Formula 1C-2-1 to Chemical Formula 1C-2-4 (e.g., according to a specific substitution position of the group represented by Chemical Formula a).

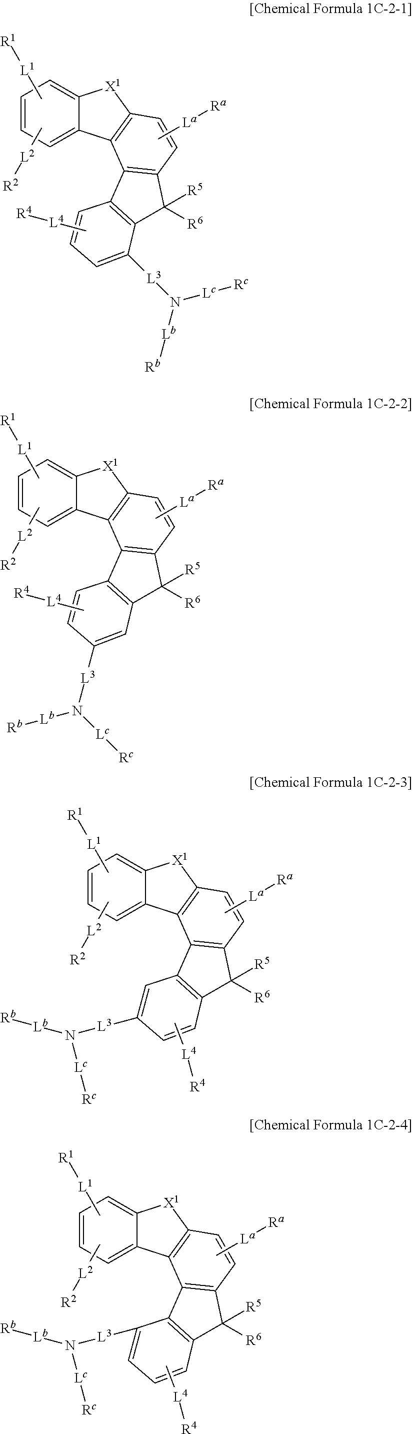

In an implementation, the first compound represented by Chemical Formula 1C may be represented by one of Chemical Formula 1C-1-1, Chemical Formula 1C-2-2, and Chemical Formula 1C-2-3.

In an implementation, the first compound represented by Chemical Formula 1D may be represented by Chemical Formula 1 D-1 or Chemical Formula 1D-2 (e.g., according to a substitution direction of the group represented by Chemical Formula a).

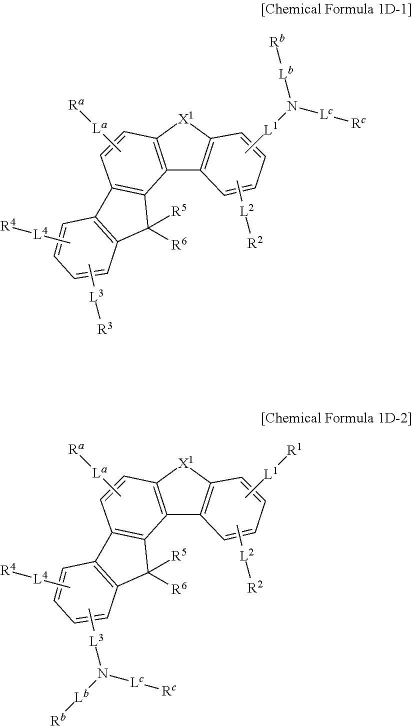

In Chemical Formula 1D-1 and Chemical Formula 1D-2, X¹, L^{a}, L^{b}, L^{c}, L¹ to L⁴, R¹ to R⁶, R^{b}, and R^{c} may be defined the same as those of Chemical Formulae 1 and 2.

In an implementation, the first compound represented by Chemical Formula 1D-1 may be represented by one of Chemical Formula 1D-1-1 to Chemical Formula 1D-1-4 (e.g., according to a specific substitution position of the group represented by Chemical Formula a).

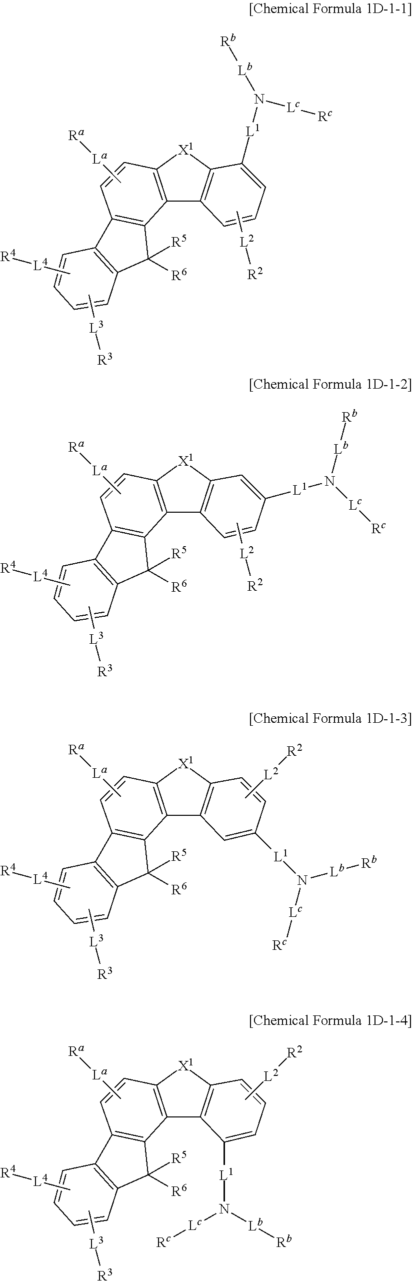

In Chemical Formula 1D-1-1 to Chemical Formula 1D-1-4, X¹, L^{a}, L^{b}, L^{c}, L¹ to L⁴, R¹ to R⁶, R^{b}, and R^{c} may be defined the same as those of Chemical Formulae 1 and 2.

In an implementation, the first compound represented by Chemical Formula 1D-2 may be represented by one of Chemical Formula 1D-2-1 to Chemical Formula 1 D-2-4 (e.g., according to a specific substitution position of the group represented by Chemical Formula a).

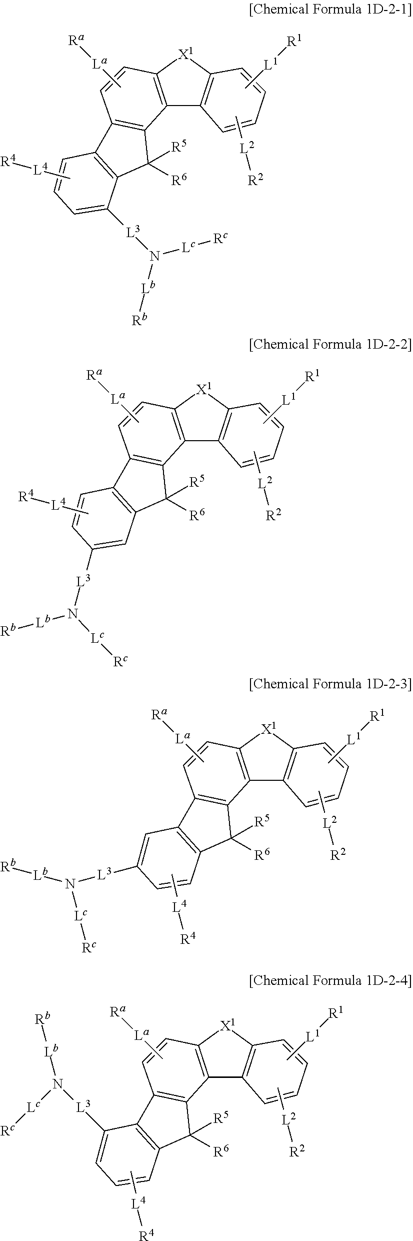

In Chemical Formula 1D-2-1 to Chemical Formula 1D-2-4, X¹, L^{a}, L^{b}, L^{c}, L¹ to L⁴, R¹ to R⁶, R^{b}, and R^{c} may be defined the same as those of Chemical Formulae 1 and 2.

In an implementation, the first compound represented by Chemical Formula 1 D may be represented by one of Chemical Formula 1 D-1-1, Chemical Formula 1 D-2-2, and Chemical Formula 1D-2-3.

In an implementation, the first compound represented by Chemical Formula 1E may be represented by one of Chemical Formula 1E-1 or Chemical Formula 1E-2 (e.g., according to a substitution direction of the group represented by Chemical Formula a).

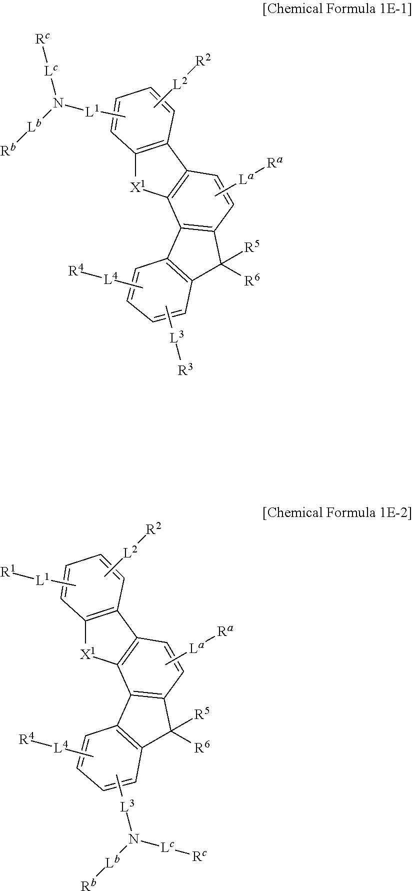

In Chemical Formula 1E-1 and Chemical Formula 1E-2, X¹, L^{a}, L^{b}, L^{c}, L¹ to L⁴, R¹ to R⁶, R^{b}, and R^{c} may be defined the same as those of Chemical Formulae 1 and 2.

In an implementation, the first compound represented by Chemical Formula 1E-1 may be represented by one of Chemical Formula 1E-1-1 to Chemical Formula 1E-1-4 (e.g., according to a specific substitution position of the group represented by Chemical Formula a).

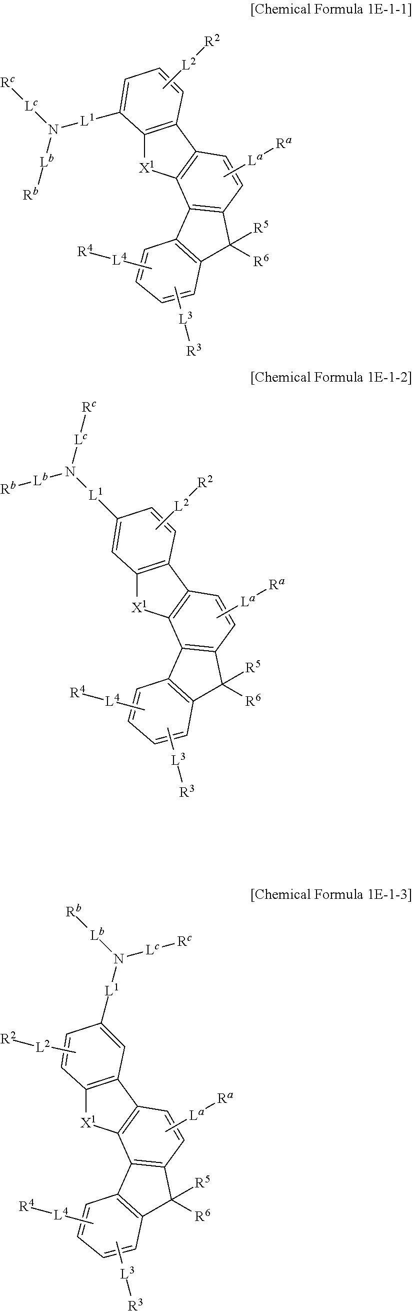

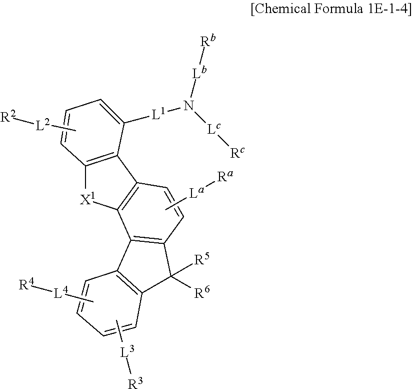

In Chemical Formula 1E-1-1 to Chemical Formula 1E-1-4, X¹, L^{a}, L^{b}, L^{c}, L¹ to L⁴, R¹ to R⁶, R^{b}, and R^{c} may be defined the same as those of Chemical Formulae 1 and 2.

In an implementation, the first compound represented by Chemical Formula 1E-2 may be represented by one of Chemical Formula 1E-2-1 to Chemical Formula 1E-2-4 (e.g., according to a specific substitution position of the group represented by Chemical Formula a).

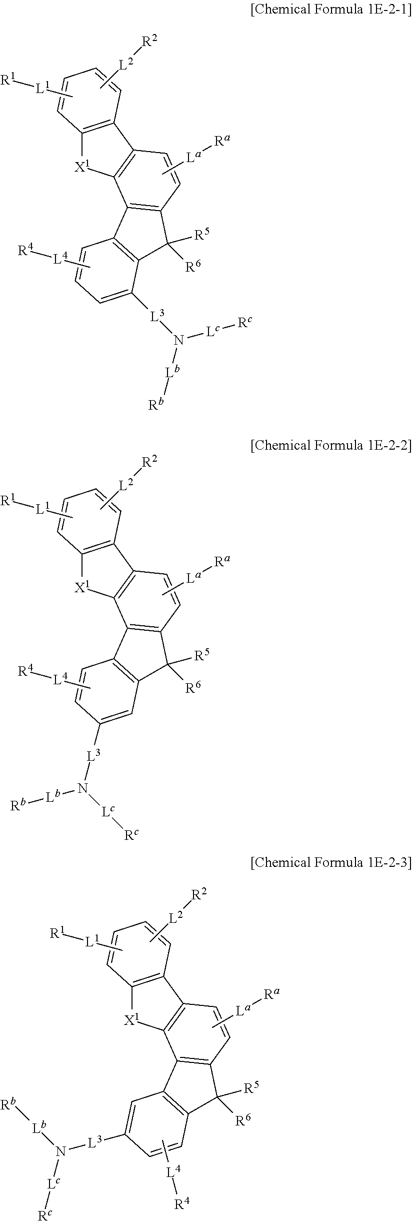

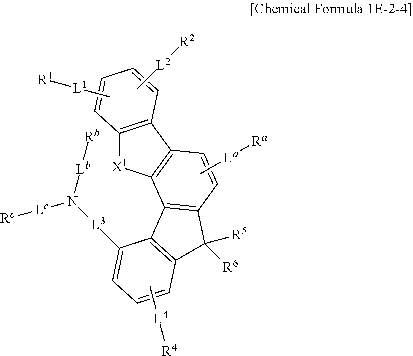

In Chemical Formula 1E-2-1 to Chemical Formula 1E-2-4, X¹, L^{a}, L^{b}, L^{c}, L¹ to L⁴, R¹ to R⁶, R^{b}, and R^{c} may be defined the same as those of Chemical Formulae 1 and 2.

In an implementation, the first compound represented by Chemical Formula 1E may be represented by one of Chemical Formula 1E-1-1 to Chemical Formula 1E-1-4, and Chemical Formula 1E-2-1 to Chemical Formula 1E-2-4.

In an implementation, the first compound represented by Chemical Formula 1F may be represented by Chemical Formula 1F-1 or Chemical Formula 1F-2 (e.g., according to a substitution direction of the group represented by Chemical Formula a).

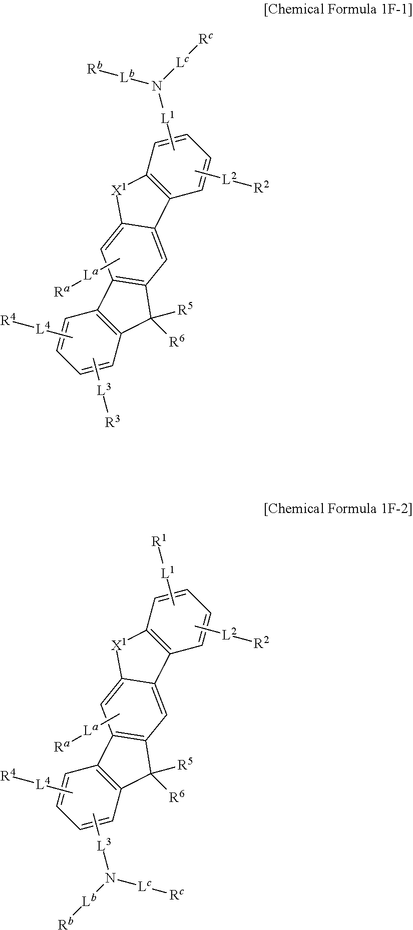

In Chemical Formula 1F-1 and Chemical Formula 1F-2, X¹, L^{a}, L^{b}, L^{c}, L¹ to L⁴, R¹ to R⁶, R^{b}, and R^{c} may be defined the same as those of Chemical Formulae 1 and 2.

In an implementation, the first compound represented by Chemical Formula 1F-1 may be represented by one of Chemical Formula 1F-1-1 to Chemical Formula 1F-1-4 (e.g., according to a specific substitution position of the group represented by Chemical Formula a).

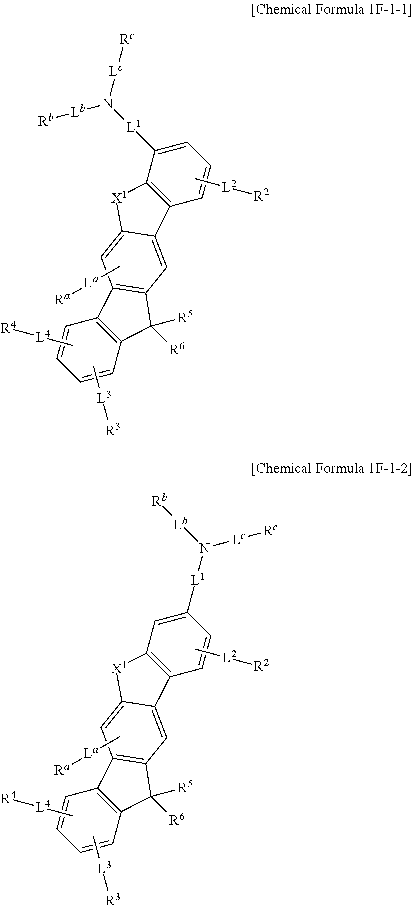

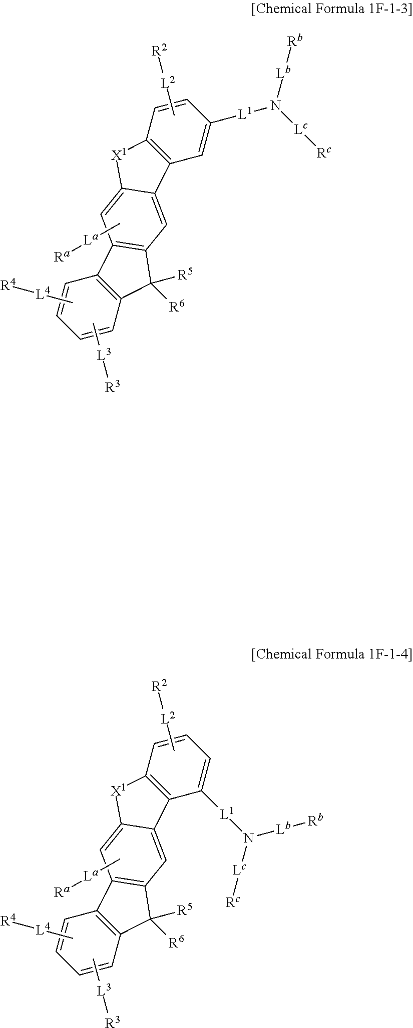

In Chemical Formula 1F-1-1 to Chemical Formula 1F-1-4, X¹, L^{a}, L^{b}, L^{c}, L¹ to L⁴, R¹ to R⁶, R^{b}, and R^{c} may be defined the same as those of Chemical Formulae 1 and 2.

In an implementation, the first compound represented by Chemical Formula 1F-2 may be represented by one of Chemical Formula 1F-2-1 to Chemical Formula 1F-2-4 (e.g., according to a specific substitution position of the group represented by Chemical Formula a).

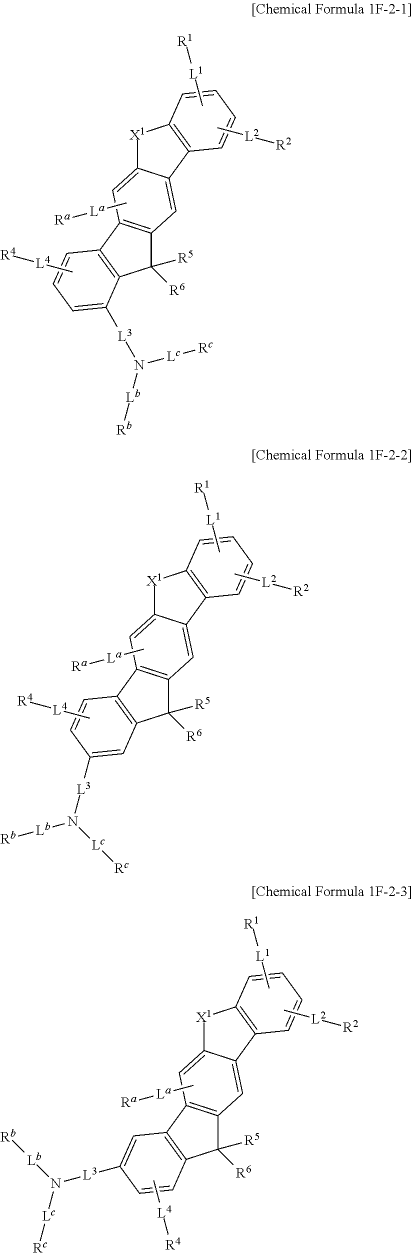

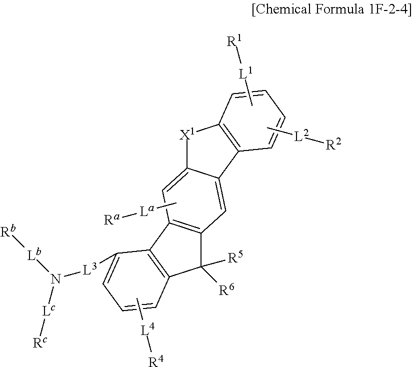

In Chemical Formula 1F-2-1 to Chemical Formula 1F-2-4, X¹, L^{a}, L^{b}, L^{c}, L¹ to L⁴, R¹ to R⁶, R^{b}, and R^{c} may be defined the same as those of Chemical Formulae 1 and 2.

In an implementation, the first compound represented by Chemical Formula 1F may be represented by one of Chemical Formula 1F-1-1, Chemical Formula 1F-2-2, and Chemical Formula 1F-2-3.

In an implementation, the first compound may be represented by Chemical Formula 1E-1-1 or Chemical Formula 1E-2-2, and may be, e.g., represented by Chemical Formula 1E-2-2.

In an implementation, the first compound may be, e.g., a compound of the following Group 1.

[Group 1]

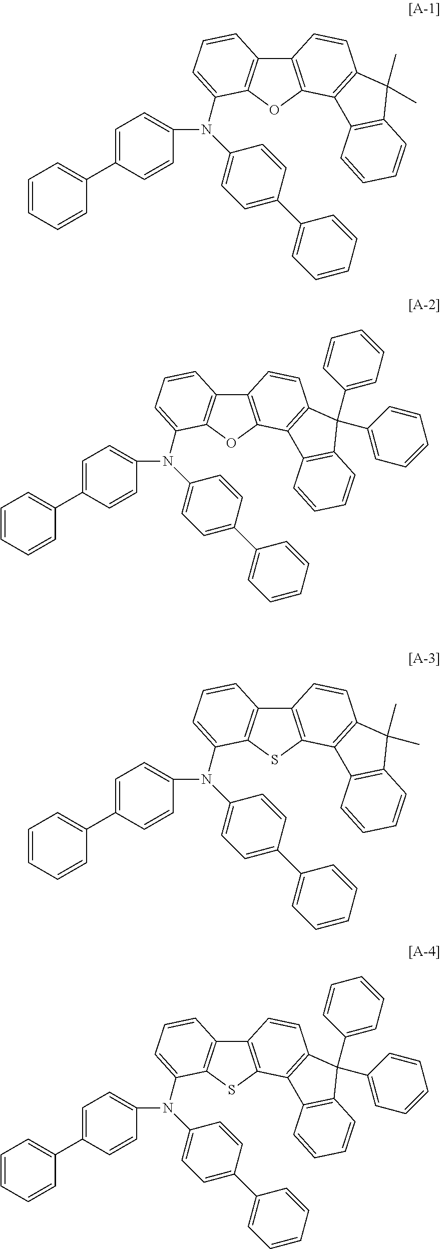 [SMILES: CC1(C)c2ccccc2-c2c1ccc1c2oc2c(N(c3ccc(-c4ccccc4)cc3)c3ccc(-c4ccccc4)cc3)cccc21 ; c1ccc(-c2ccc(N(c3ccc(-c4ccccc4)cc3)c3cccc4c3oc3c5c(ccc34)C(c3ccccc3)(c3ccccc3)c3ccccc3-5)cc2)cc1 ; CC1(C)c2ccccc2-c2c1ccc1c2sc2c(N(c3ccc(-c4ccccc4)cc3)c3ccc(-c4ccccc4)cc3)cccc21 ; c1ccc(-c2ccc(N(c3ccc(-c4ccccc4)cc3)c3cccc4c3sc3c5c(ccc34)C(c3ccccc3)(c3ccccc3)c3ccccc3-5)cc2)cc1]

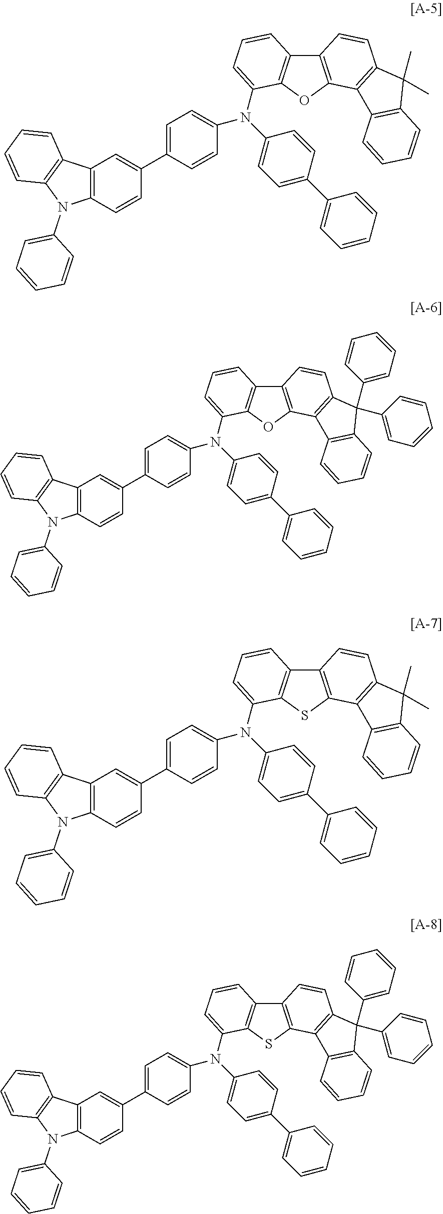 [SMILES: CC1(C)c2ccccc2-c2c1ccc1c2oc2c(N(c3ccc(-c4ccccc4)cc3)c3ccc(-c4ccc5c(c4)c4ccccc4n5-c4ccccc4)cc3)cccc21 ; c1ccc(-c2ccc(N(c3ccc(-c4ccc5c(c4)c4ccccc4n5-c4ccccc4)cc3)c3cccc4c3oc3c5c(ccc34)C(c3ccccc3)(c3ccccc3)c3ccccc3-5)cc2)cc1 ; CC1(C)c2ccccc2-c2c1ccc1c2sc2c(N(c3ccc(-c4ccccc4)cc3)c3ccc(-c4ccc5c(c4)c4ccccc4n5-c4ccccc4)cc3)cccc21 ; c1ccc(-c2ccc(N(c3ccc(-c4ccc5c(c4)c4ccccc4n5-c4ccccc4)cc3)c3cccc4c3sc3c5c(ccc34)C(c3ccccc3)(c3ccccc3)c3ccccc3-5)cc2)cc1]

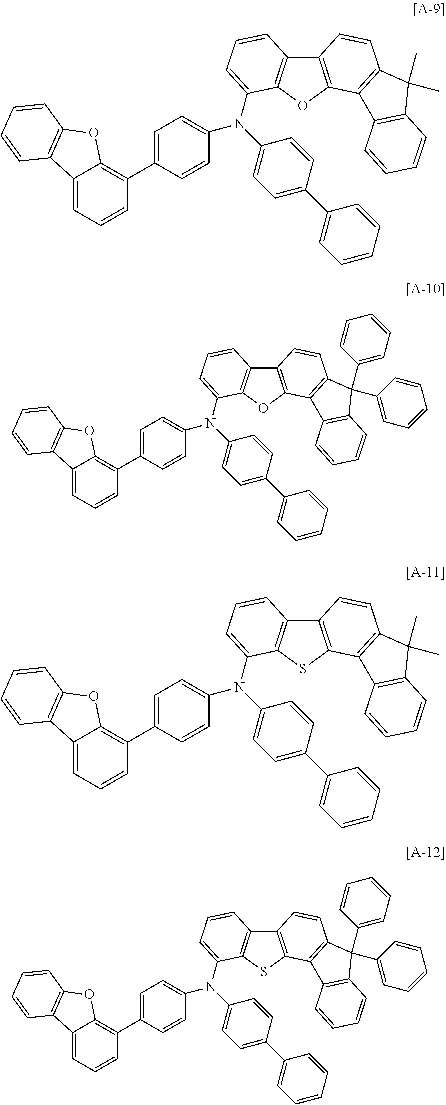 [SMILES: CC1(C)c2ccccc2-c2c1ccc1c2oc2c(N(c3ccc(-c4ccccc4)cc3)c3ccc(-c4cccc5c4oc4ccccc45)cc3)cccc21 ; c1ccc(-c2ccc(N(c3ccc(-c4cccc5c4oc4ccccc45)cc3)c3cccc4c3oc3c5c(ccc34)C(c3ccccc3)(c3ccccc3)c3ccccc3-5)cc2)cc1 ; CC1(C)c2ccccc2-c2c1ccc1c2sc2c(N(c3ccc(-c4ccccc4)cc3)c3ccc(-c4cccc5c4oc4ccccc45)cc3)cccc21 ; c1ccc(-c2ccc(N(c3ccc(-c4cccc5c4oc4ccccc45)cc3)c3cccc4c3sc3c5c(ccc34)C(c3ccccc3)(c3ccccc3)c3ccccc3-5)cc2)cc1]

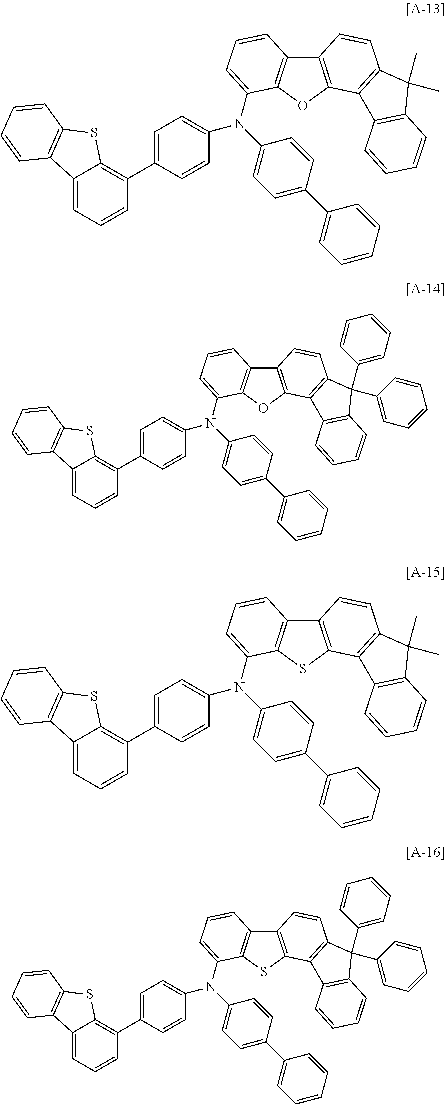 [SMILES: CC1(C)c2ccccc2-c2c1ccc1c2oc2c(N(c3ccc(-c4ccccc4)cc3)c3ccc(-c4cccc5c4sc4ccccc45)cc3)cccc21 ; c1ccc(-c2ccc(N(c3ccc(-c4cccc5c4sc4ccccc45)cc3)c3cccc4c3oc3c5c(ccc34)C(c3ccccc3)(c3ccccc3)c3ccccc3-5)cc2)cc1 ; CC1(C)c2ccccc2-c2c1ccc1c2sc2c(N(c3ccc(-c4ccccc4)cc3)c3ccc(-c4cccc5c4sc4ccccc45)cc3)cccc21 ; c1ccc(-c2ccc(N(c3ccc(-c4cccc5c4sc4ccccc45)cc3)c3cccc4c3sc3c5c(ccc34)C(c3ccccc3)(c3ccccc3)c3ccccc3-5)cc2)cc1]

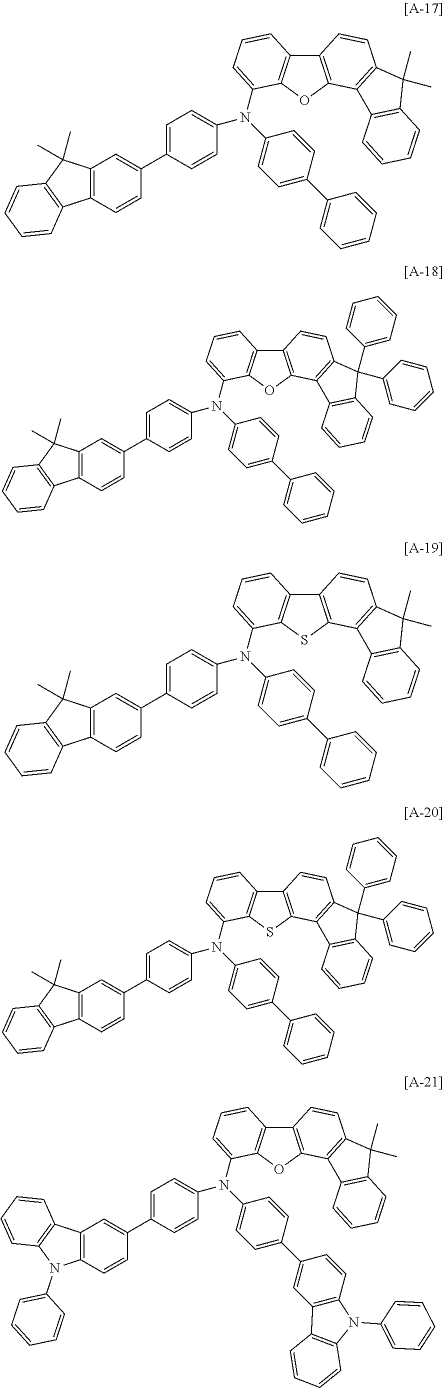

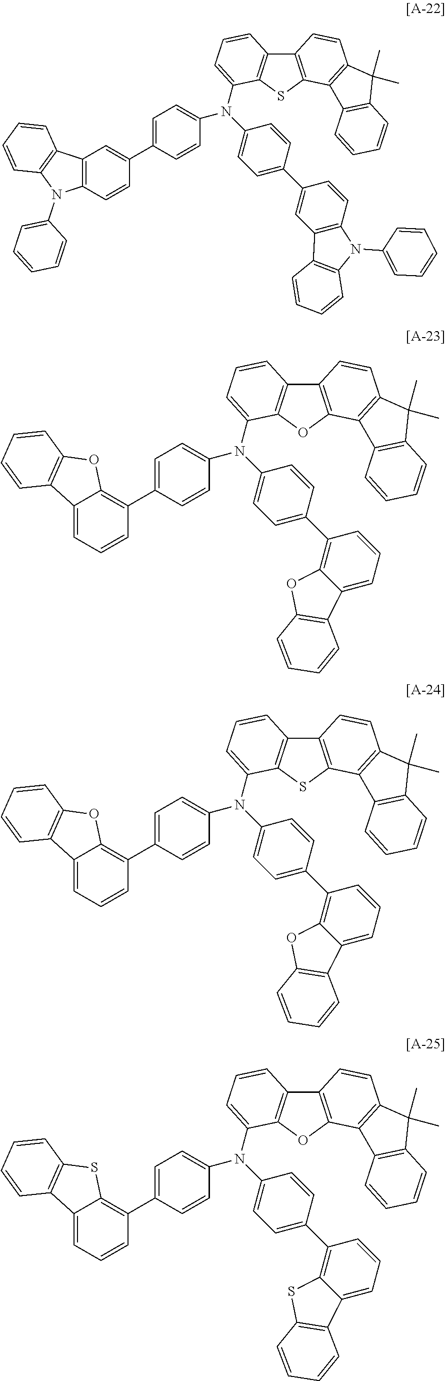 [SMILES: CC1(C)c2ccccc2-c2c1ccc1c2sc2c(N(c3ccc(-c4ccc5c(c4)c4ccccc4n5-c4ccccc4)cc3)c3ccc(-c4ccc5c(c4)c4ccccc4n5-c4ccccc4)cc3)cccc21 ; CC1(C)c2ccccc2-c2c1ccc1c2oc2c(N(c3ccc(-c4cccc5c4oc4ccccc45)cc3)c3ccc(-c4cccc5c4oc4ccccc45)cc3)cccc21 ; CC1(C)c2ccccc2-c2c1ccc1c2sc2c(N(c3ccc(-c4cccc5c4oc4ccccc45)cc3)c3ccc(-c4cccc5c4oc4ccccc45)cc3)cccc21 ; CC1(C)c2ccccc2-c2c1ccc1c2oc2c(N(c3ccc(-c4cccc5c4sc4ccccc45)cc3)c3ccc(-c4cccc5c4sc4ccccc45)cc3)cccc21]

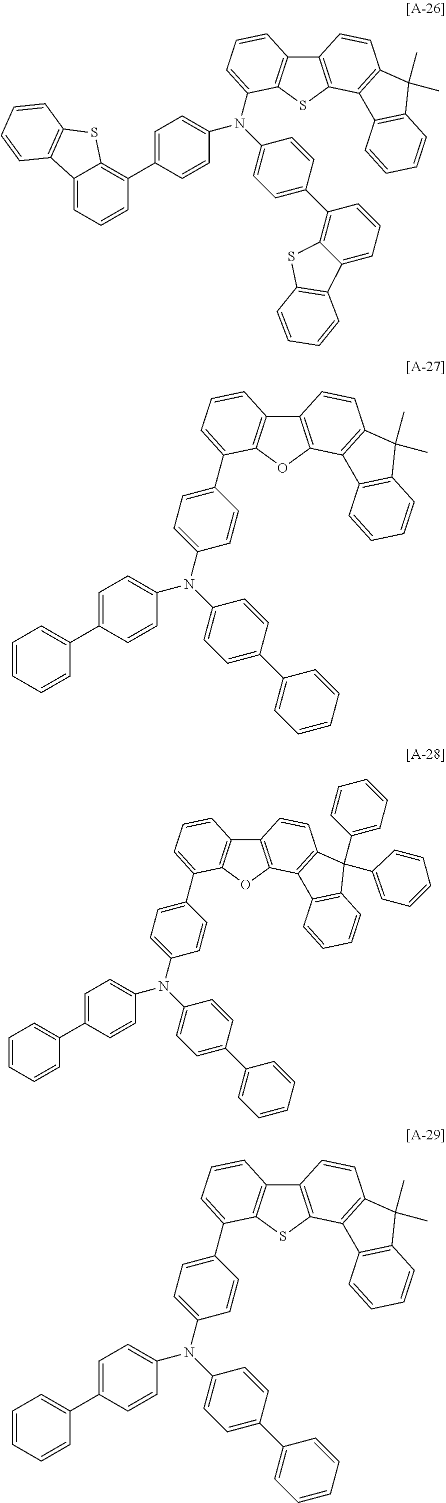 [SMILES: CC1(C)c2ccccc2-c2c1ccc1c2sc2c(N(c3ccc(-c4cccc5c4sc4ccccc45)cc3)c3ccc(-c4cccc5c4sc4ccccc45)cc3)cccc21 ; CC1(C)c2ccccc2-c2c1ccc1c2oc2c(-c3ccc(N(c4ccc(-c5ccccc5)cc4)c4ccc(-c5ccccc5)cc4)cc3)cccc21 ; c1ccc(-c2ccc(N(c3ccc(-c4ccccc4)cc3)c3ccc(-c4cccc5c4oc4c6c(ccc45)C(c4ccccc4)(c4ccccc4)c4ccccc4-6)cc3)cc2)cc1 ; CC1(C)c2ccccc2-c2c1ccc1c2sc2c(-c3ccc(N(c4ccc(-c5ccccc5)cc4)c4ccc(-c5ccccc5)cc4)cc3)cccc21]

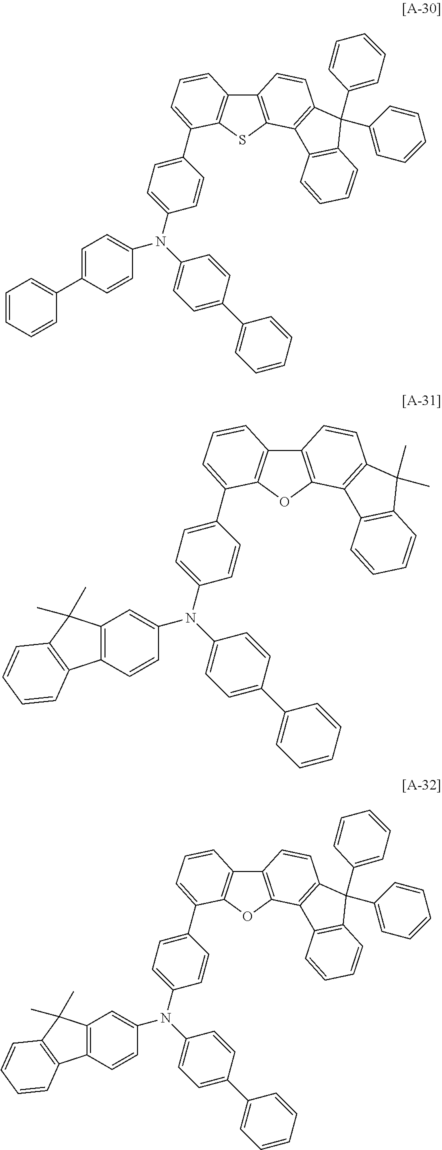 [SMILES: c1ccc(-c2ccc(N(c3ccc(-c4ccccc4)cc3)c3ccc(-c4cccc5c4sc4c6c(ccc45)C(c4ccccc4)(c4ccccc4)c4ccccc4-6)cc3)cc2)cc1 ; CC1(C)c2ccccc2-c2ccc(N(c3ccc(-c4ccccc4)cc3)c3ccc(-c4cccc5c4oc4c6c(ccc45)C(C)(C)c4ccccc4-6)cc3)cc21 ; CC1(C)c2ccccc2-c2ccc(N(c3ccc(-c4ccccc4)cc3)c3ccc(-c4cccc5c4oc4c6c(ccc45)C(c4ccccc4)(c4ccccc4)c4ccccc4-6)cc3)cc21]

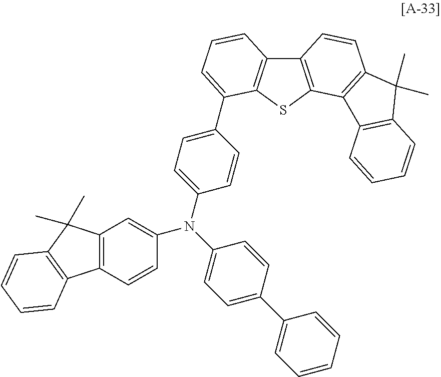 [SMILES: CC1(C)c2ccccc2-c2ccc(N(c3ccc(-c4ccccc4)cc3)c3ccc(-c4cccc5c4sc4c6c(ccc45)C(C)(C)c4ccccc4-6)cc3)cc21]

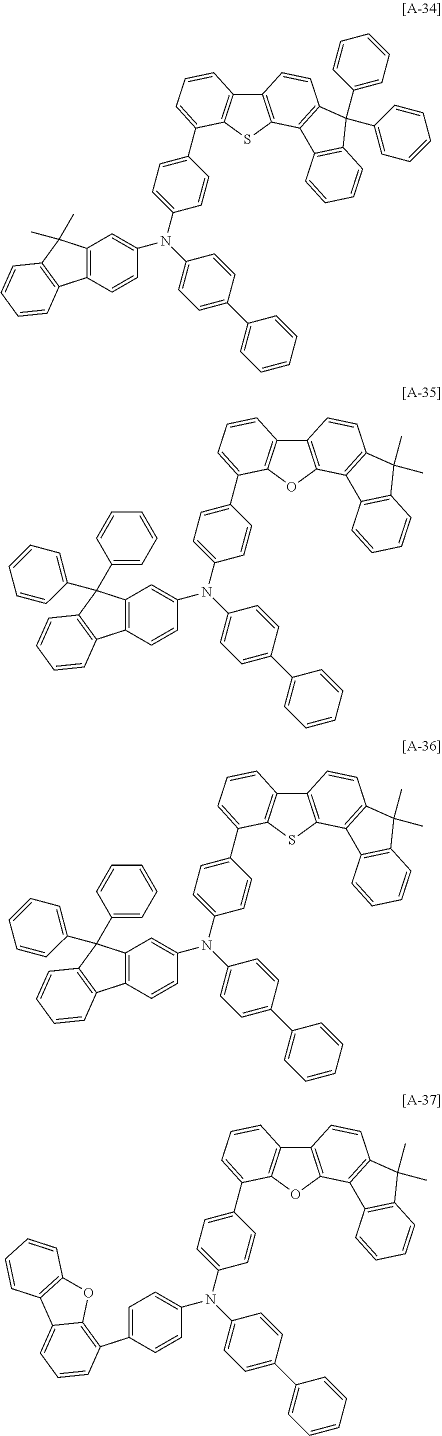 [SMILES: CC1(C)c2ccccc2-c2ccc(N(c3ccc(-c4ccccc4)cc3)c3ccc(-c4cccc5c4sc4c6c(ccc45)C(c4ccccc4)(c4ccccc4)c4ccccc4-6)cc3)cc21 ; CC1(C)c2ccccc2-c2c1ccc1c2oc2c(-c3ccc(N(c4ccc(-c5ccccc5)cc4)c4ccc5c(c4)C(c4ccccc4)(c4ccccc4)c4ccccc4-5)cc3)cccc21 ; CC1(C)c2ccccc2-c2c1ccc1c2sc2c(-c3ccc(N(c4ccc(-c5ccccc5)cc4)c4ccc5c(c4)C(c4ccccc4)(c4ccccc4)c4ccccc4-5)cc3)cccc21 ; CC1(C)c2ccccc2-c2c1ccc1c2oc2c(-c3ccc(N(c4ccc(-c5ccccc5)cc4)c4ccc(-c5cccc6c5oc5ccccc56)cc4)cc3)cccc21]

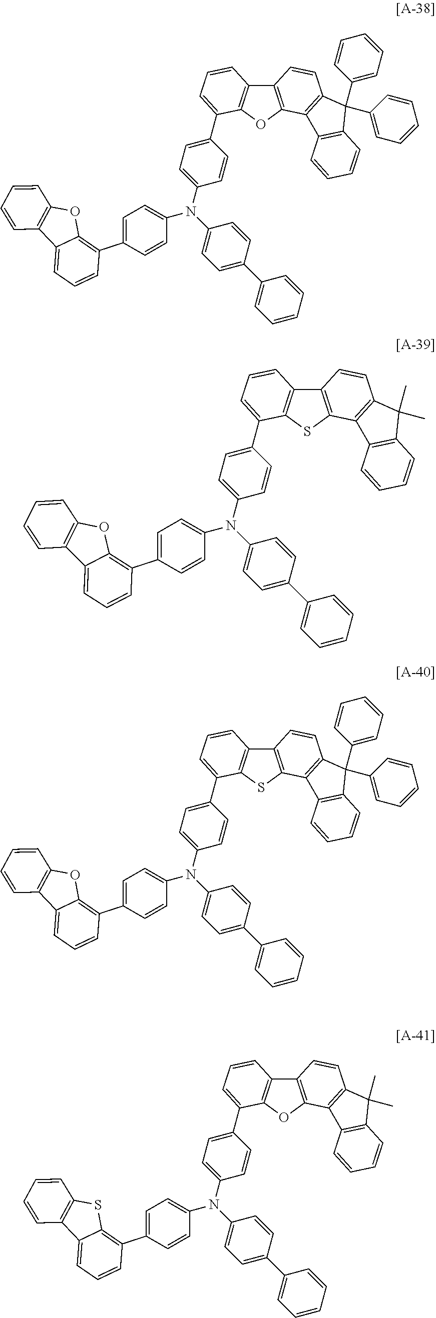 [SMILES: c1ccc(-c2ccc(N(c3ccc(-c4cccc5c4oc4ccccc45)cc3)c3ccc(-c4cccc5c4oc4c6c(ccc45)C(c4ccccc4)(c4ccccc4)c4ccccc4-6)cc3)cc2)cc1 ; CC1(C)c2ccccc2-c2c1ccc1c2sc2c(-c3ccc(N(c4ccc(-c5ccccc5)cc4)c4ccc(-c5cccc6c5oc5ccccc56)cc4)cc3)cccc21 ; c1ccc(-c2ccc(N(c3ccc(-c4cccc5c4oc4ccccc45)cc3)c3ccc(-c4cccc5c4sc4c6c(ccc45)C(c4ccccc4)(c4ccccc4)c4ccccc4-6)cc3)cc2)cc1 ; CC1(C)c2ccccc2-c2c1ccc1c2oc2c(-c3ccc(N(c4ccc(-c5ccccc5)cc4)c4ccc(-c5cccc6c5sc5ccccc56)cc4)cc3)cccc21]

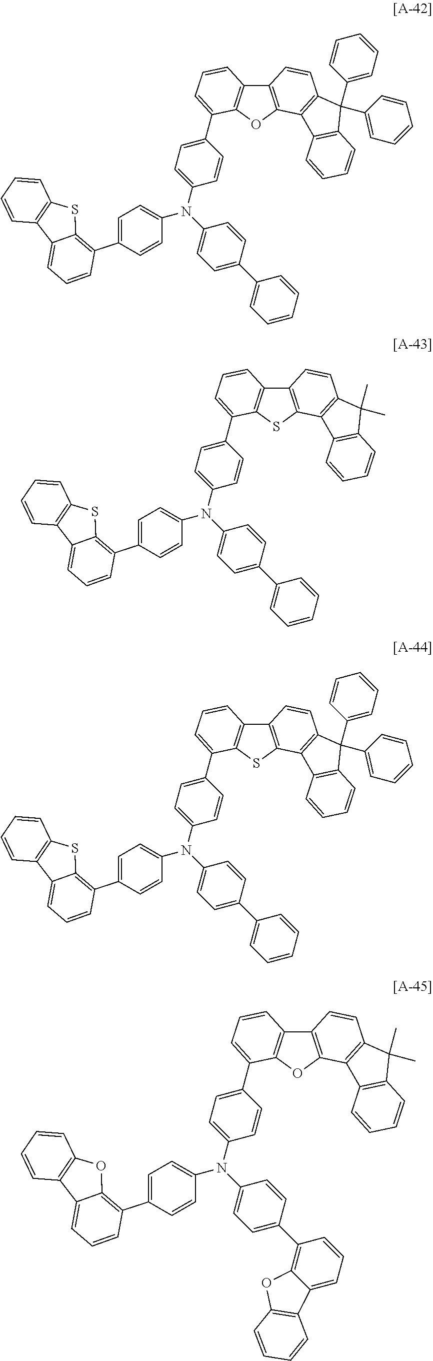 [SMILES: c1ccc(-c2ccc(N(c3ccc(-c4cccc5c4oc4c6c(ccc45)C(c4ccccc4)(c4ccccc4)c4ccccc4-6)cc3)c3ccc(-c4cccc5c4sc4ccccc45)cc3)cc2)cc1 ; CC1(C)c2ccccc2-c2c1ccc1c2sc2c(-c3ccc(N(c4ccc(-c5ccccc5)cc4)c4ccc(-c5cccc6c5sc5ccccc56)cc4)cc3)cccc21 ; c1ccc(-c2ccc(N(c3ccc(-c4cccc5c4sc4ccccc45)cc3)c3ccc(-c4cccc5c4sc4c6c(ccc45)C(c4ccccc4)(c4ccccc4)c4ccccc4-6)cc3)cc2)cc1 ; CC1(C)c2ccccc2-c2c1ccc1c2oc2c(-c3ccc(N(c4ccc(-c5cccc6c5oc5ccccc56)cc4)c4ccc(-c5cccc6c5oc5ccccc56)cc4)cc3)cccc21]

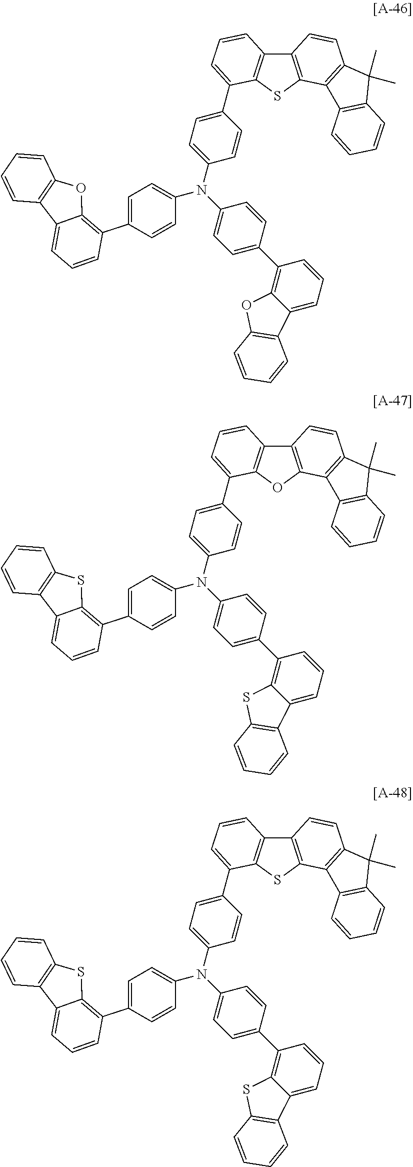 [SMILES: CC1(C)c2ccccc2-c2c1ccc1c2sc2c(-c3ccc(N(c4ccc(-c5cccc6c5oc5ccccc56)cc4)c4ccc(-c5cccc6c5oc5ccccc56)cc4)cc3)cccc21 ; CC1(C)c2ccccc2-c2c1ccc1c2oc2c(-c3ccc(N(c4ccc(-c5cccc6c5sc5ccccc56)cc4)c4ccc(-c5cccc6c5sc5ccccc56)cc4)cc3)cccc21 ; CC1(C)c2ccccc2-c2c1ccc1c2sc2c(-c3ccc(N(c4ccc(-c5cccc6c5sc5ccccc56)cc4)c4ccc(-c5cccc6c5sc5ccccc56)cc4)cc3)cccc21]

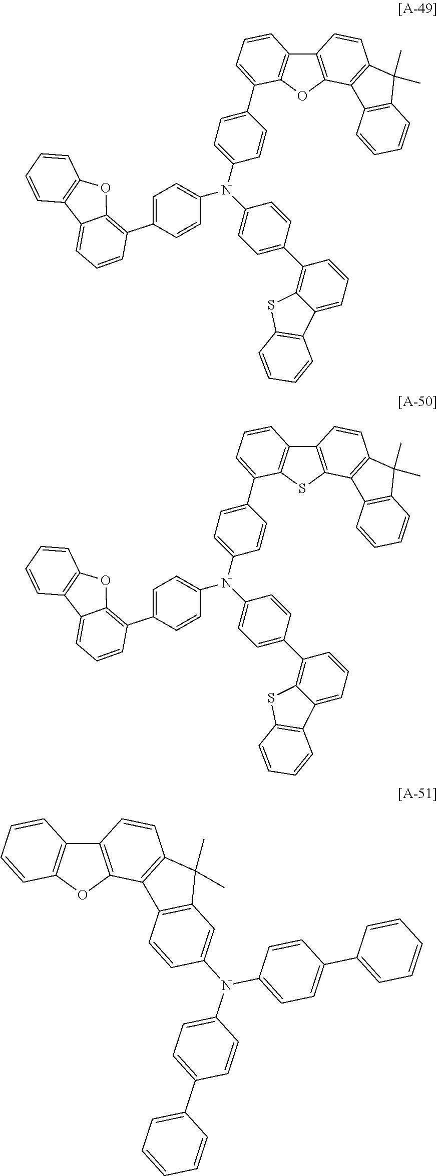 [SMILES: CC1(C)c2ccccc2-c2c1ccc1c2oc2c(-c3ccc(N(c4ccc(-c5cccc6c5oc5ccccc56)cc4)c4ccc(-c5cccc6c5sc5ccccc56)cc4)cc3)cccc21 ; CC1(C)c2ccccc2-c2c1ccc1c2sc2c(-c3ccc(N(c4ccc(-c5cccc6c5oc5ccccc56)cc4)c4ccc(-c5cccc6c5sc5ccccc56)cc4)cc3)cccc21 ; CC1(C)c2cc(N(c3ccc(-c4ccccc4)cc3)c3ccc(-c4ccccc4)cc3)ccc2-c2c1ccc1c2oc2ccccc21]

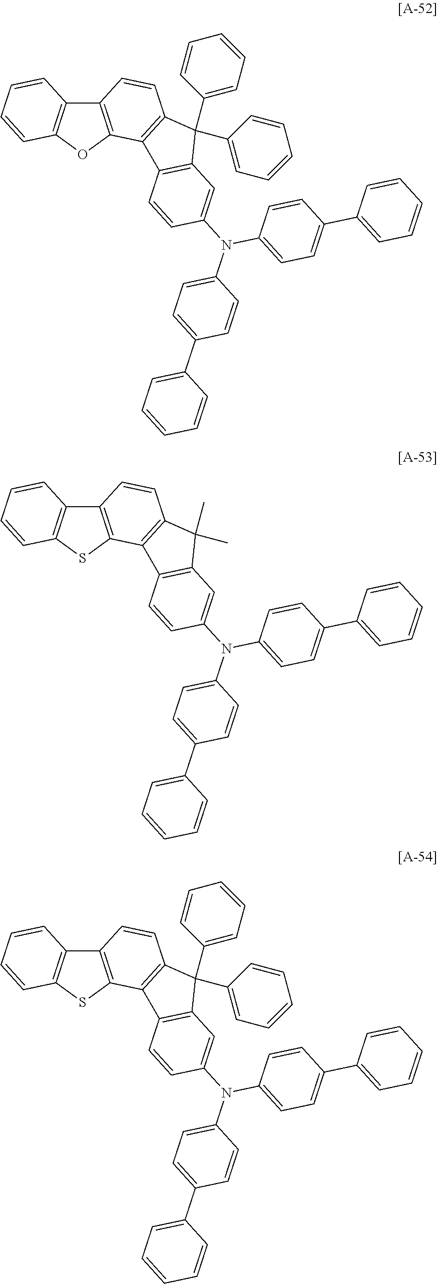 [SMILES: c1ccc(-c2ccc(N(c3ccc(-c4ccccc4)cc3)c3ccc4c(c3)C(c3ccccc3)(c3ccccc3)c3ccc5c(oc6ccccc65)c3-4)cc2)cc1 ; CC1(C)c2cc(N(c3ccc(-c4ccccc4)cc3)c3ccc(-c4ccccc4)cc3)ccc2-c2c1ccc1c2sc2ccccc21 ; c1ccc(-c2ccc(N(c3ccc(-c4ccccc4)cc3)c3ccc4c(c3)C(c3ccccc3)(c3ccccc3)c3ccc5c(sc6ccccc65)c3-4)cc2)cc1]

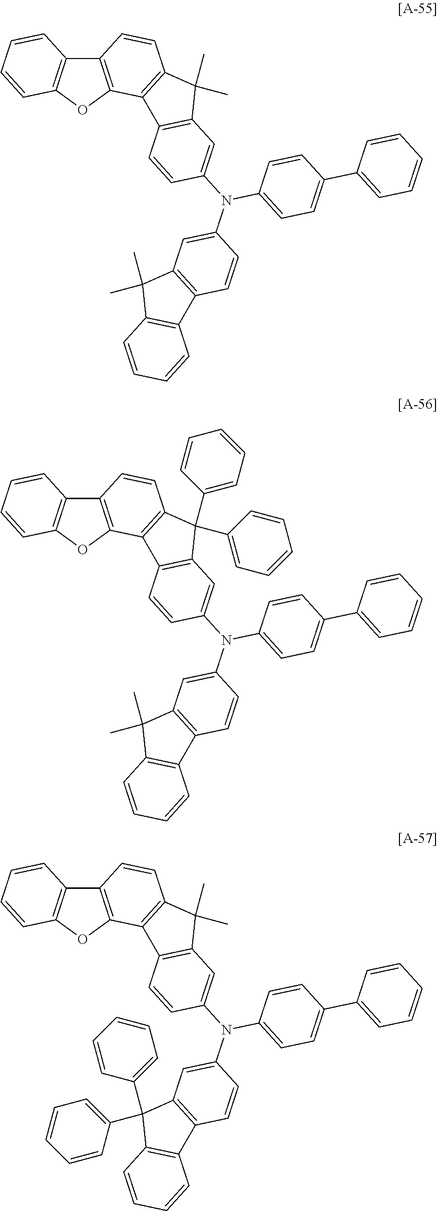 [SMILES: CC1(C)c2ccccc2-c2ccc(N(c3ccc(-c4ccccc4)cc3)c3ccc4c(c3)C(C)(C)c3ccc5c(oc6ccccc65)c3-4)cc21 ; CC1(C)c2ccccc2-c2ccc(N(c3ccc(-c4ccccc4)cc3)c3ccc4c(c3)C(c3ccccc3)(c3ccccc3)c3ccc5c(oc6ccccc65)c3-4)cc21 ; CC1(C)c2cc(N(c3ccc(-c4ccccc4)cc3)c3ccc4c(c3)C(c3ccccc3)(c3ccccc3)c3ccccc3-4)ccc2-c2c1ccc1c2oc2ccccc21]

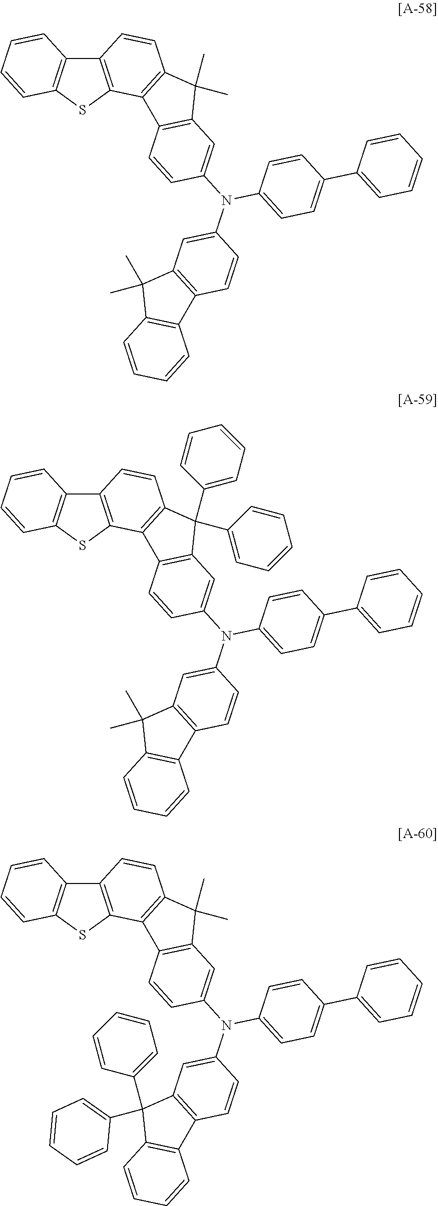 [SMILES: CC1(C)c2ccccc2-c2ccc(N(c3ccc(-c4ccccc4)cc3)c3ccc4c(c3)C(C)(C)c3ccc5c(sc6ccccc65)c3-4)cc21 ; CC1(C)c2ccccc2-c2ccc(N(c3ccc(-c4ccccc4)cc3)c3ccc4c(c3)C(c3ccccc3)(c3ccccc3)c3ccc5c(sc6ccccc65)c3-4)cc21 ; CC1(C)c2cc(N(c3ccc(-c4ccccc4)cc3)c3ccc4c(c3)C(c3ccccc3)(c3ccccc3)c3ccccc3-4)ccc2-c2c1ccc1c2sc2ccccc21]

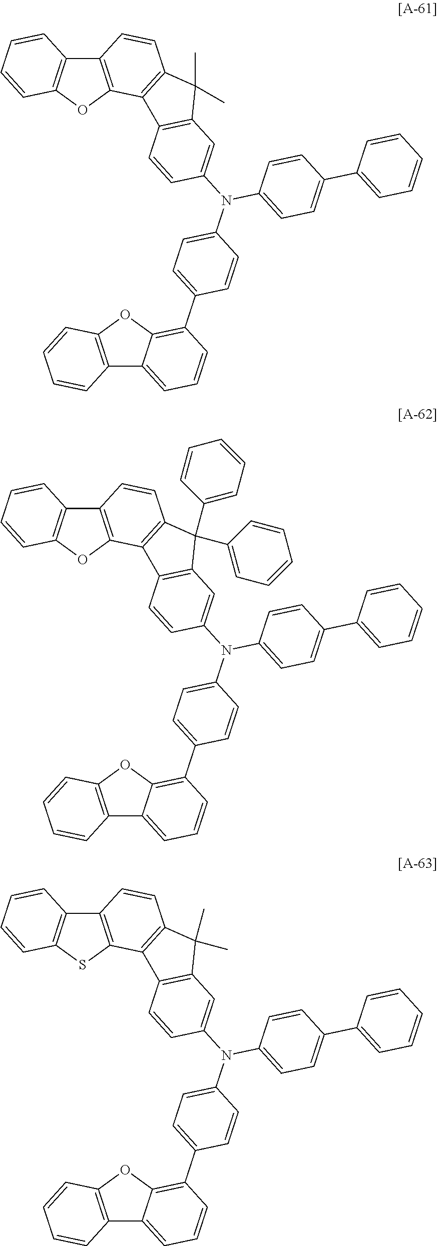 [SMILES: CC1(C)c2cc(N(c3ccc(-c4ccccc4)cc3)c3ccc(-c4cccc5c4oc4ccccc45)cc3)ccc2-c2c1ccc1c2oc2ccccc21 ; c1ccc(-c2ccc(N(c3ccc(-c4cccc5c4oc4ccccc45)cc3)c3ccc4c(c3)C(c3ccccc3)(c3ccccc3)c3ccc5c(oc6ccccc65)c3-4)cc2)cc1 ; CC1(C)c2cc(N(c3ccc(-c4ccccc4)cc3)c3ccc(-c4cccc5c4oc4ccccc45)cc3)ccc2-c2c1ccc1c2sc2ccccc21]

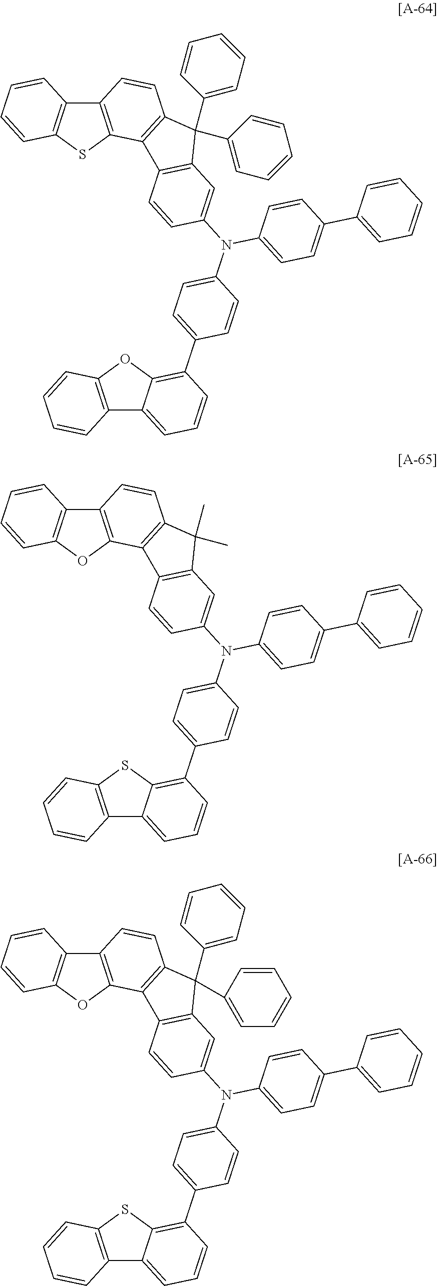 [SMILES: c1ccc(-c2ccc(N(c3ccc(-c4cccc5c4oc4ccccc45)cc3)c3ccc4c(c3)C(c3ccccc3)(c3ccccc3)c3ccc5c(sc6ccccc65)c3-4)cc2)cc1 ; CC1(C)c2cc(N(c3ccc(-c4ccccc4)cc3)c3ccc(-c4cccc5c4sc4ccccc45)cc3)ccc2-c2c1ccc1c2oc2ccccc21 ; c1ccc(-c2ccc(N(c3ccc(-c4cccc5c4sc4ccccc45)cc3)c3ccc4c(c3)C(c3ccccc3)(c3ccccc3)c3ccc5c(oc6ccccc65)c3-4)cc2)cc1]

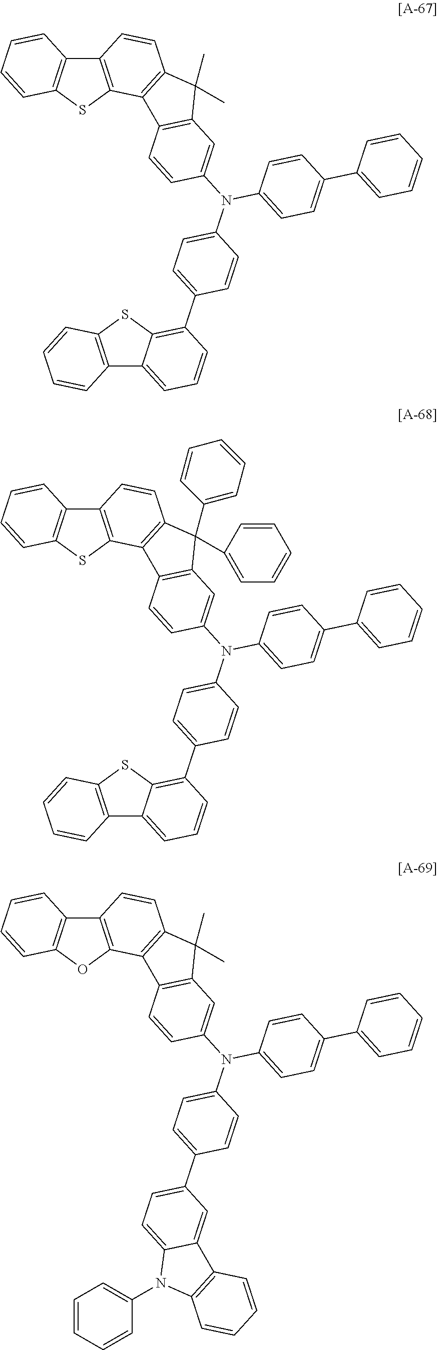 [SMILES: CC1(C)c2cc(N(c3ccc(-c4ccccc4)cc3)c3ccc(-c4cccc5c4sc4ccccc45)cc3)ccc2-c2c1ccc1c2sc2ccccc21 ; c1ccc(-c2ccc(N(c3ccc(-c4cccc5c4sc4ccccc45)cc3)c3ccc4c(c3)C(c3ccccc3)(c3ccccc3)c3ccc5c(sc6ccccc65)c3-4)cc2)cc1 ; CC1(C)c2cc(N(c3ccc(-c4ccccc4)cc3)c3ccc(-c4ccc5c(c4)c4ccccc4n5-c4ccccc4)cc3)ccc2-c2c1ccc1c2oc2ccccc21]

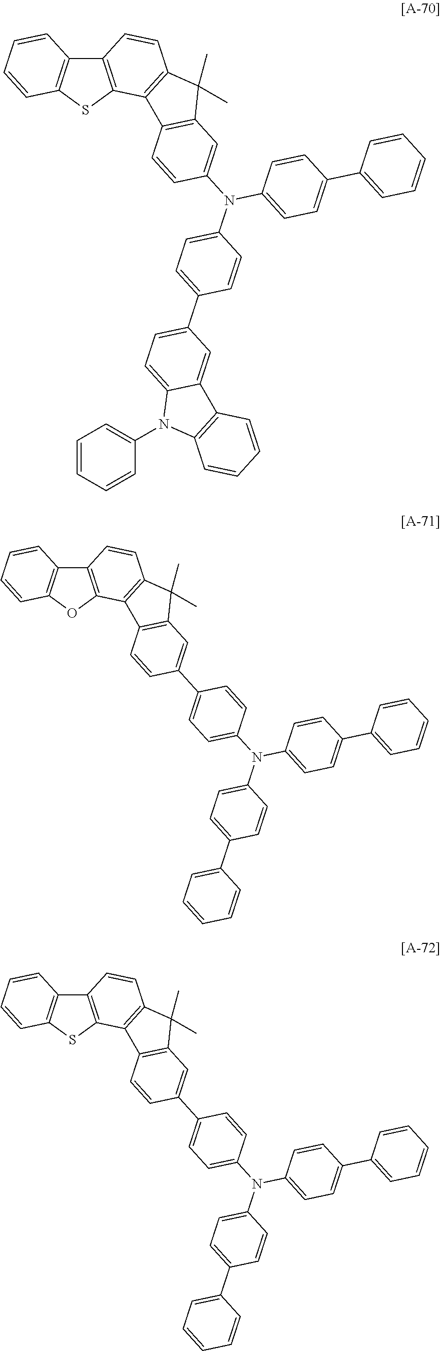 [SMILES: CC1(C)c2cc(N(c3ccc(-c4ccccc4)cc3)c3ccc(-c4ccc5c(c4)c4ccccc4n5-c4ccccc4)cc3)ccc2-c2c1ccc1c2sc2ccccc21 ; CC1(C)c2cc(-c3ccc(N(c4ccc(-c5ccccc5)cc4)c4ccc(-c5ccccc5)cc4)cc3)ccc2-c2c1ccc1c2oc2ccccc21 ; CC1(C)c2cc(-c3ccc(N(c4ccc(-c5ccccc5)cc4)c4ccc(-c5ccccc5)cc4)cc3)ccc2-c2c1ccc1c2sc2ccccc21]

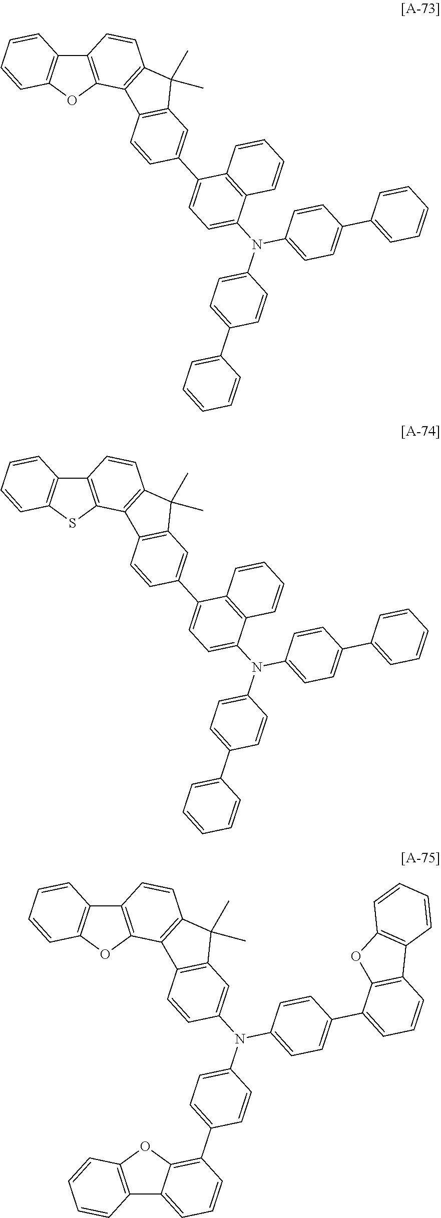 [SMILES: CC1(C)c2cc(-c3ccc(N(c4ccc(-c5ccccc5)cc4)c4ccc(-c5ccccc5)cc4)c4ccccc34)ccc2-c2c1ccc1c2oc2ccccc21 ; CC1(C)c2cc(-c3ccc(N(c4ccc(-c5ccccc5)cc4)c4ccc(-c5ccccc5)cc4)c4ccccc34)ccc2-c2c1ccc1c2sc2ccccc21 ; CC1(C)c2cc(N(c3ccc(-c4cccc5c4oc4ccccc45)cc3)c3ccc(-c4cccc5c4oc4ccccc45)cc3)ccc2-c2c1ccc1c2oc2ccccc21]

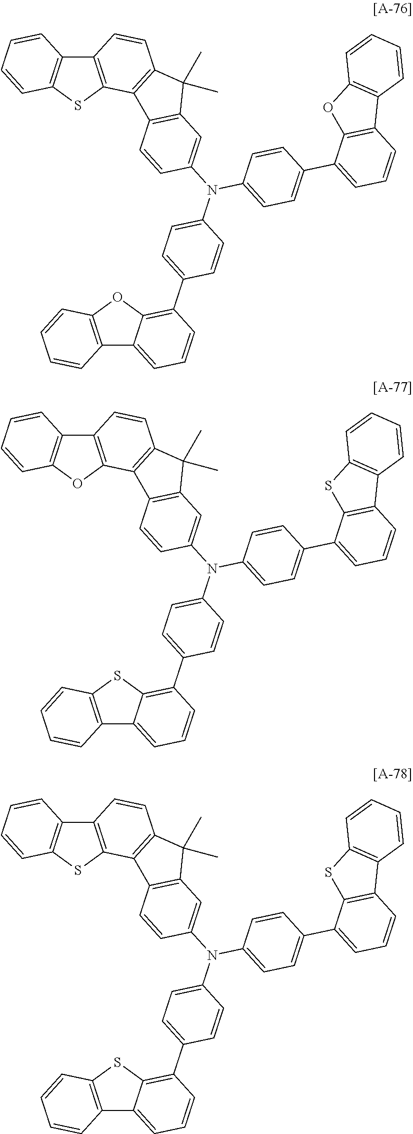 [SMILES: CC1(C)c2cc(N(c3ccc(-c4cccc5c4oc4ccccc45)cc3)c3ccc(-c4cccc5c4oc4ccccc45)cc3)ccc2-c2c1ccc1c2sc2ccccc21 ; CC1(C)c2cc(N(c3ccc(-c4cccc5c4sc4ccccc45)cc3)c3ccc(-c4cccc5c4sc4ccccc45)cc3)ccc2-c2c1ccc1c2oc2ccccc21 ; CC1(C)c2cc(N(c3ccc(-c4cccc5c4sc4ccccc45)cc3)c3ccc(-c4cccc5c4sc4ccccc45)cc3)ccc2-c2c1ccc1c2sc2ccccc21]

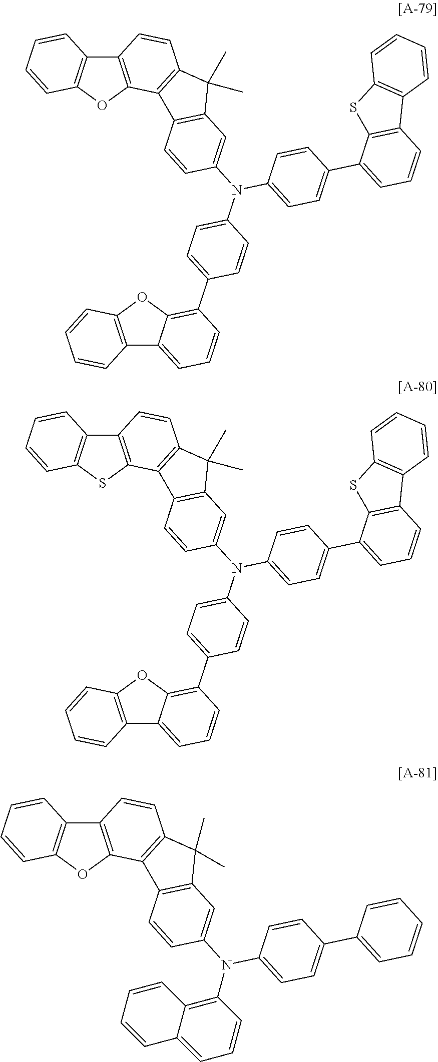 [SMILES: CC1(C)c2cc(N(c3ccc(-c4cccc5c4oc4ccccc45)cc3)c3ccc(-c4cccc5c4sc4ccccc45)cc3)ccc2-c2c1ccc1c2oc2ccccc21 ; CC1(C)c2cc(N(c3ccc(-c4cccc5c4oc4ccccc45)cc3)c3ccc(-c4cccc5c4sc4ccccc45)cc3)ccc2-c2c1ccc1c2sc2ccccc21 ; CC1(C)c2cc(N(c3ccc(-c4ccccc4)cc3)c3cccc4ccccc34)ccc2-c2c1ccc1c2oc2ccccc21]

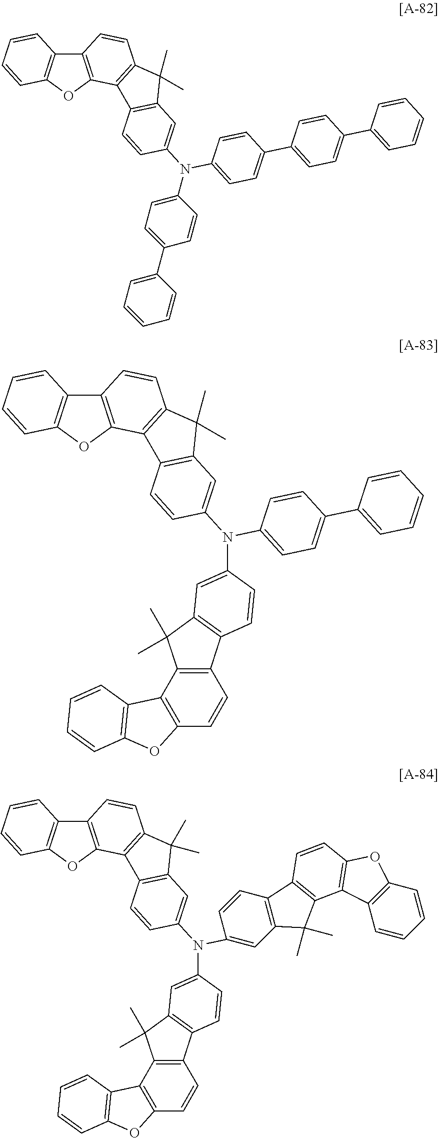 [SMILES: CC1(C)c2cc(N(c3ccc(-c4ccccc4)cc3)c3ccc(-c4ccc(-c5ccccc5)cc4)cc3)ccc2-c2c1ccc1c2oc2ccccc21 ; CC1(C)c2cc(N(c3ccc(-c4ccccc4)cc3)c3ccc4c(c3)C(C)(C)c3c-4ccc4oc5ccccc5c34)ccc2-c2c1ccc1c2oc2ccccc21 ; CC1(C)c2cc(N(c3ccc4c(c3)C(C)(C)c3c-4ccc4oc5ccccc5c34)c3ccc4c(c3)C(C)(C)c3c-4ccc4oc5ccccc5c34)ccc2-c2c1ccc1c2oc2ccccc21]

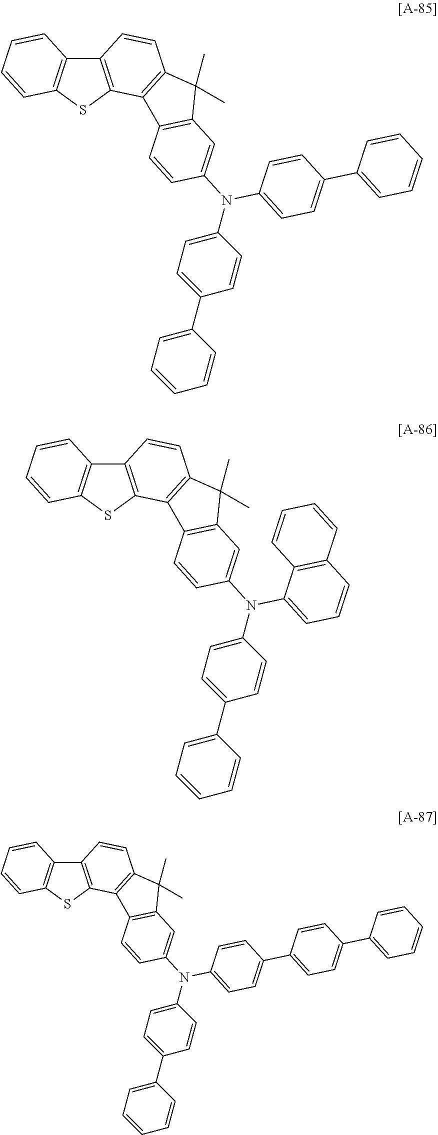 [SMILES: CC1(C)c2cc(N(c3ccc(-c4ccccc4)cc3)c3ccc(-c4ccccc4)cc3)ccc2-c2c1ccc1c2sc2ccccc21 ; CC1(C)c2cc(N(c3ccc(-c4ccccc4)cc3)c3cccc4ccccc34)ccc2-c2c1ccc1c2sc2ccccc21 ; CC1(C)c2cc(N(c3ccc(-c4ccccc4)cc3)c3ccc(-c4ccc(-c5ccccc5)cc4)cc3)ccc2-c2c1ccc1c2sc2ccccc21]

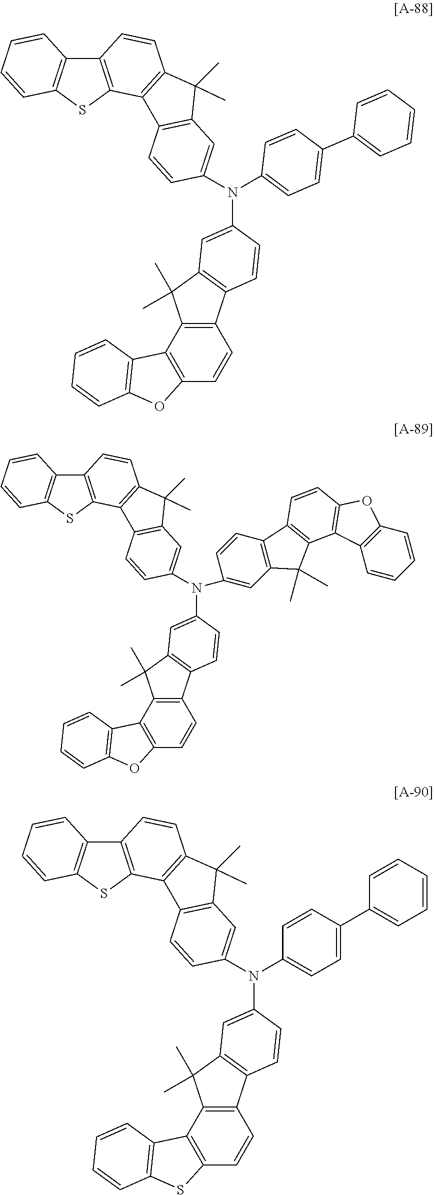 [SMILES: CC1(C)c2cc(N(c3ccc(-c4ccccc4)cc3)c3ccc4c(c3)C(C)(C)c3c-4ccc4oc5ccccc5c34)ccc2-c2c1ccc1c2sc2ccccc21 ; CC1(C)c2cc(N(c3ccc4c(c3)C(C)(C)c3c-4ccc4oc5ccccc5c34)c3ccc4c(c3)C(C)(C)c3c-4ccc4oc5ccccc5c34)ccc2-c2c1ccc1c2sc2ccccc21 ; CC1(C)c2cc(N(c3ccc(-c4ccccc4)cc3)c3ccc4c(c3)C(C)(C)c3c-4ccc4sc5ccccc5c34)ccc2-c2c1ccc1c2sc2ccccc21]

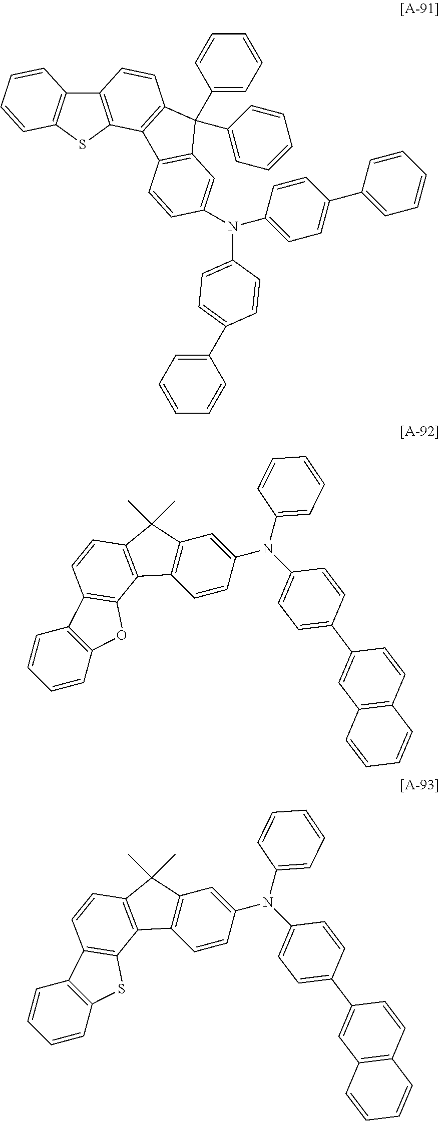 [SMILES: c1ccc(-c2ccc(N(c3ccc(-c4ccccc4)cc3)c3ccc4c(c3)C(c3ccccc3)(c3ccccc3)c3ccc5c(sc6ccccc65)c3-4)cc2)cc1 ; CC1(C)c2cc(N(c3ccccc3)c3ccc(-c4ccc5ccccc5c4)cc3)ccc2-c2c1ccc1c2oc2ccccc21 ; CC1(C)c2cc(N(c3ccccc3)c3ccc(-c4ccc5ccccc5c4)cc3)ccc2-c2c1ccc1c2sc2ccccc21]

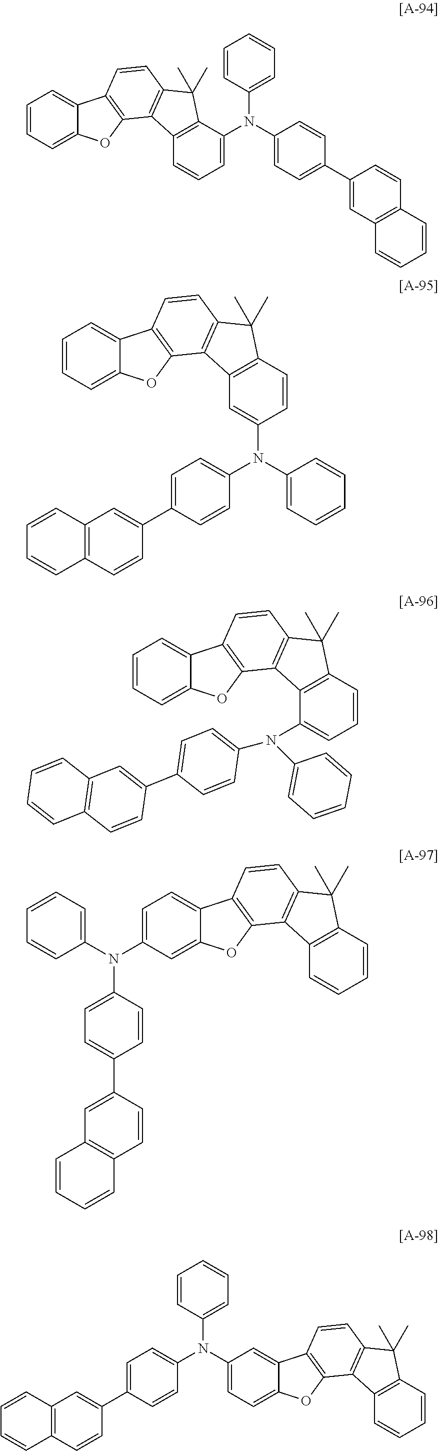 [SMILES: CC1(C)c2ccc3c(oc4ccccc43)c2-c2cccc(N(c3ccccc3)c3ccc(-c4ccc5ccccc5c4)cc3)c21 ; CC1(C)c2ccc(N(c3ccccc3)c3ccc(-c4ccc5ccccc5c4)cc3)cc2-c2c1ccc1c2oc2ccccc21 ; CC1(C)c2cccc(N(c3ccccc3)c3ccc(-c4ccc5ccccc5c4)cc3)c2-c2c1ccc1c2oc2ccccc21 ; CC1(C)c2ccccc2-c2c1ccc1c2oc2cc(N(c3ccccc3)c3ccc(-c4ccc5ccccc5c4)cc3)ccc21 ; CC1(C)c2ccccc2-c2c1ccc1c2oc2ccc(N(c3ccccc3)c3ccc(-c4ccc5ccccc5c4)cc3)cc21]

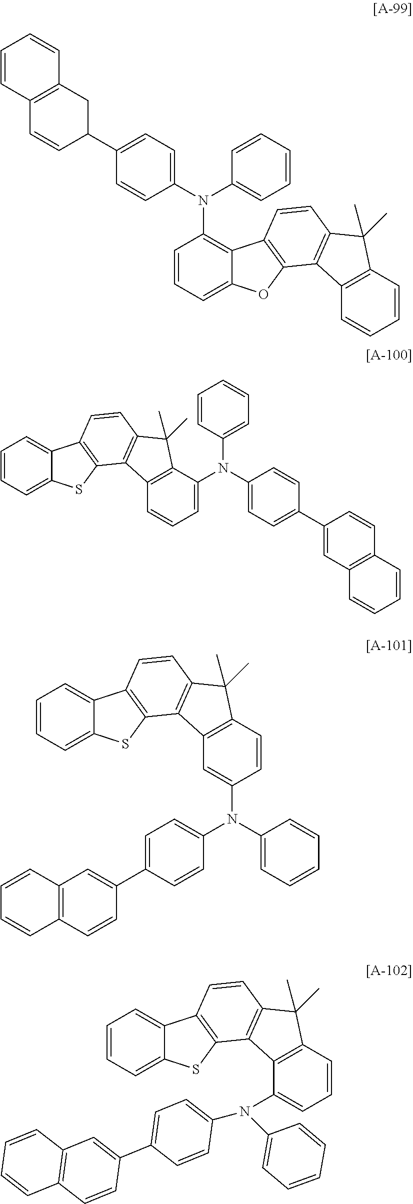 [SMILES: CC1(C)c2ccccc2-c2c1ccc1c2oc2cccc(N(c3ccccc3)c3ccc(C4C=Cc5ccccc5C4)cc3)c21 ; CC1(C)c2ccc3c(sc4ccccc43)c2-c2cccc(N(c3ccccc3)c3ccc(-c4ccc5ccccc5c4)cc3)c21 ; CC1(C)c2ccc(N(c3ccccc3)c3ccc(-c4ccc5ccccc5c4)cc3)cc2-c2c1ccc1c2sc2ccccc21 ; CC1(C)c2cccc(N(c3ccccc3)c3ccc(-c4ccc5ccccc5c4)cc3)c2-c2c1ccc1c2sc2ccccc21]

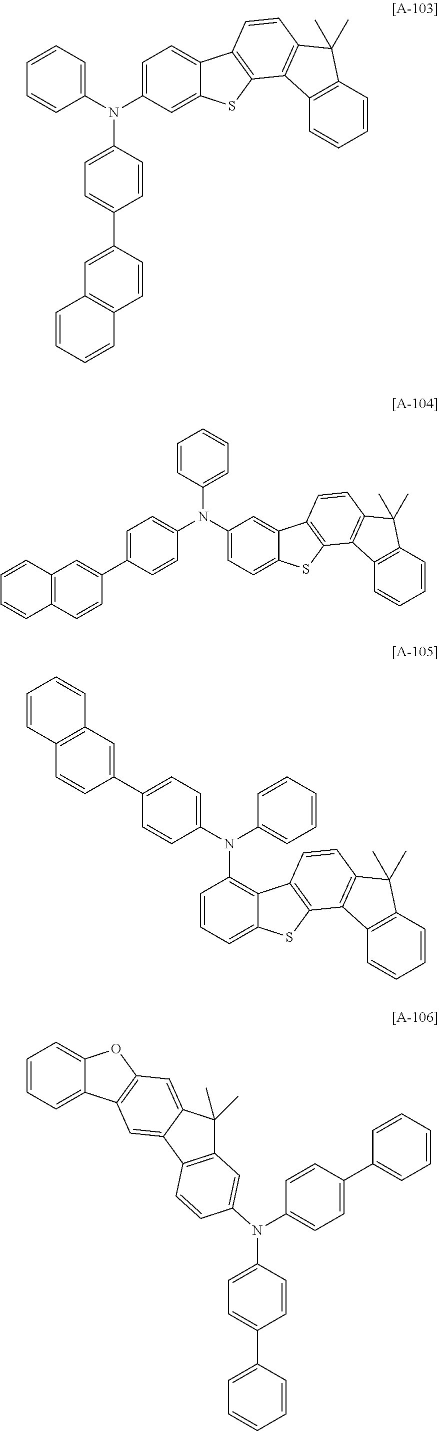 [SMILES: CC1(C)c2ccccc2-c2c1ccc1c2sc2cc(N(c3ccccc3)c3ccc(-c4ccc5ccccc5c4)cc3)ccc21 ; CC1(C)c2ccccc2-c2c1ccc1c2sc2ccc(N(c3ccccc3)c3ccc(-c4ccc5ccccc5c4)cc3)cc21 ; CC1(C)c2ccccc2-c2c1ccc1c2sc2cccc(N(c3ccccc3)c3ccc(-c4ccc5ccccc5c4)cc3)c21 ; CC1(C)c2cc(N(c3ccc(-c4ccccc4)cc3)c3ccc(-c4ccccc4)cc3)ccc2-c2cc3c(cc21)oc1ccccc13]

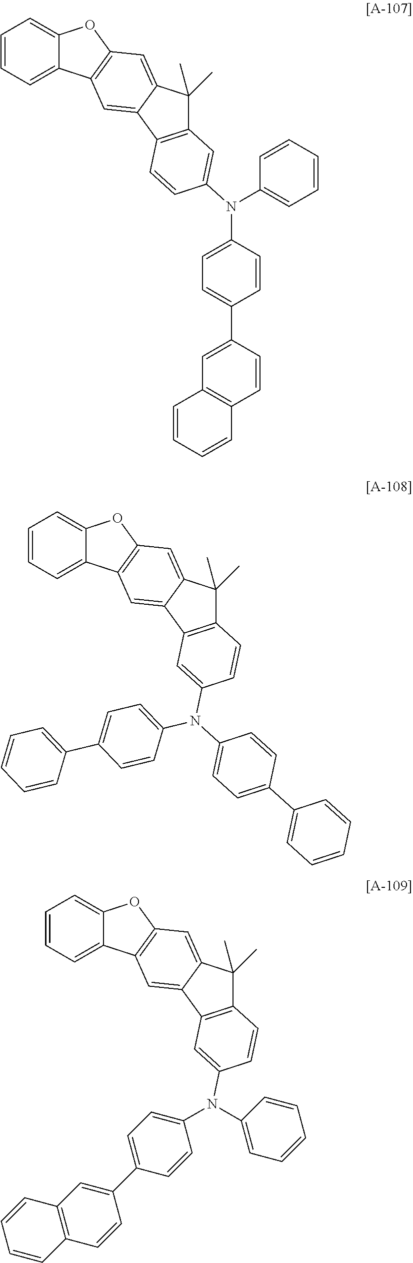 [SMILES: CC1(C)c2cc(N(c3ccccc3)c3ccc(-c4ccc5ccccc5c4)cc3)ccc2-c2cc3c(cc21)oc1ccccc13 ; CC1(C)c2ccc(N(c3ccc(-c4ccccc4)cc3)c3ccc(-c4ccccc4)cc3)cc2-c2cc3c(cc21)oc1ccccc13 ; CC1(C)c2ccc(N(c3ccccc3)c3ccc(-c4ccc5ccccc5c4)cc3)cc2-c2cc3c(cc21)oc1ccccc13]

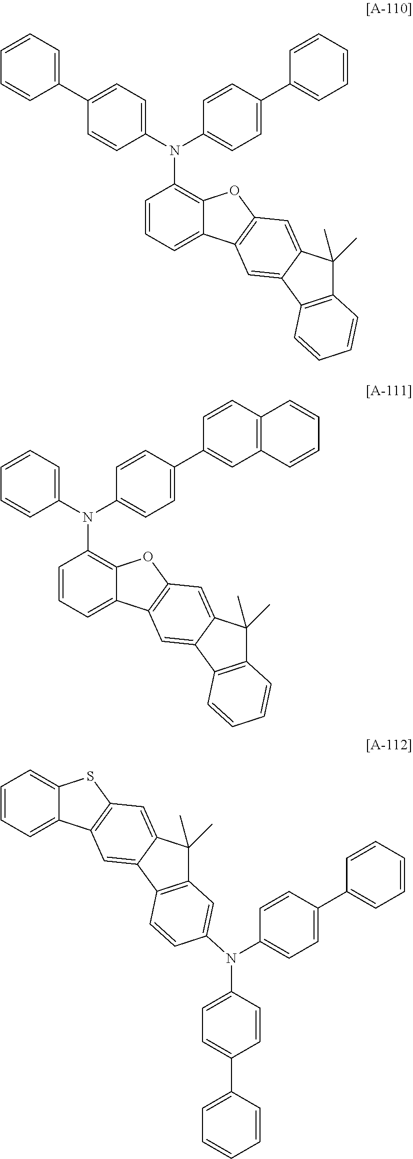 [SMILES: CC1(C)c2ccccc2-c2cc3c(cc21)oc1c(N(c2ccc(-c4ccccc4)cc2)c2ccc(-c4ccccc4)cc2)cccc13 ; CC1(C)c2ccccc2-c2cc3c(cc21)oc1c(N(c2ccccc2)c2ccc(-c4ccc5ccccc5c4)cc2)cccc13 ; CC1(C)c2cc(N(c3ccc(-c4ccccc4)cc3)c3ccc(-c4ccccc4)cc3)ccc2-c2cc3c(cc21)sc1ccccc13]

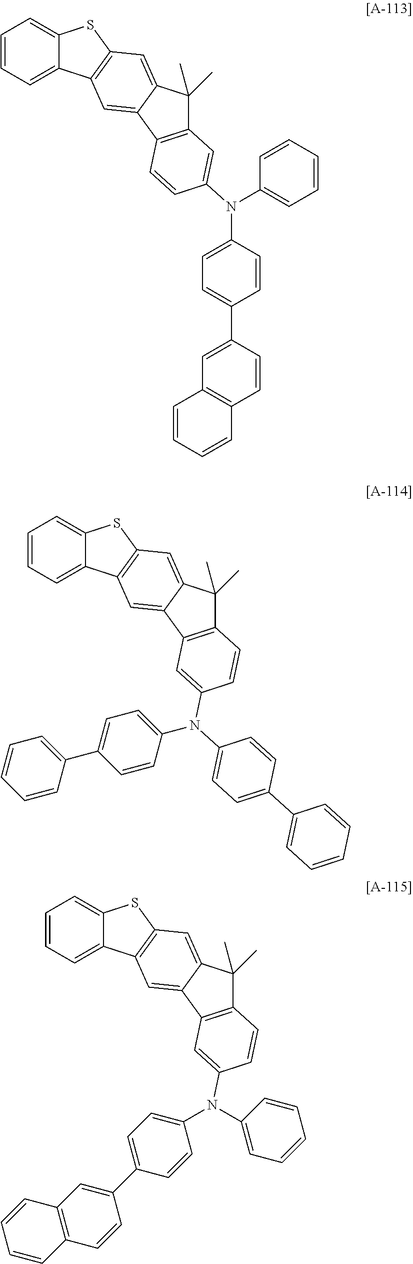 [SMILES: CC1(C)c2cc(N(c3ccccc3)c3ccc(-c4ccc5ccccc5c4)cc3)ccc2-c2cc3c(cc21)sc1ccccc13 ; CC1(C)c2ccc(N(c3ccc(-c4ccccc4)cc3)c3ccc(-c4ccccc4)cc3)cc2-c2cc3c(cc21)sc1ccccc13 ; CC1(C)c2ccc(N(c3ccccc3)c3ccc(-c4ccc5ccccc5c4)cc3)cc2-c2cc3c(cc21)sc1ccccc13]

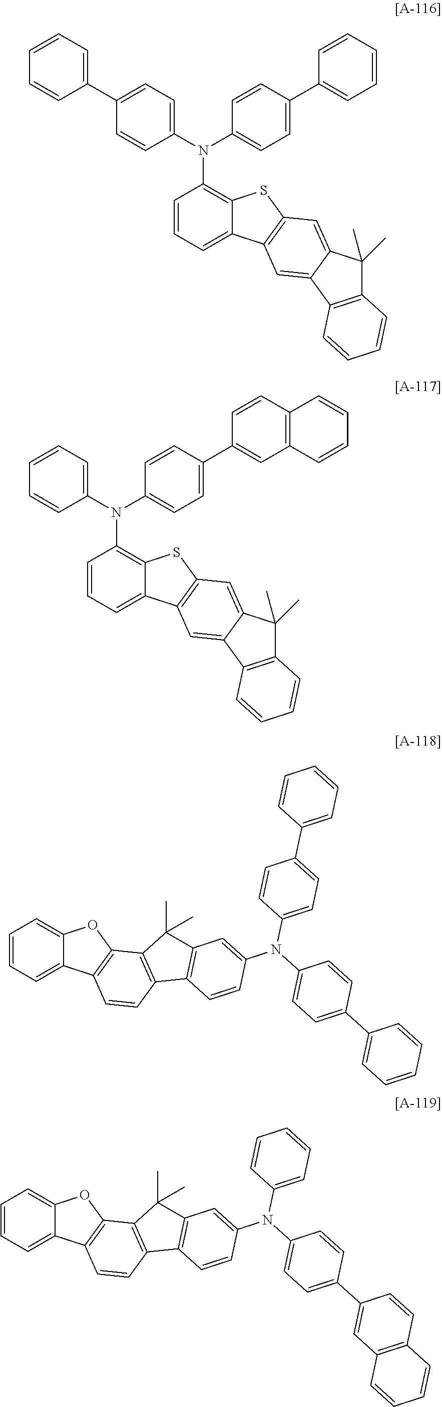 [SMILES: CC1(C)c2ccccc2-c2cc3c(cc21)sc1c(N(c2ccc(-c4ccccc4)cc2)c2ccc(-c4ccccc4)cc2)cccc13 ; CC1(C)c2ccccc2-c2cc3c(cc21)sc1c(N(c2ccccc2)c2ccc(-c4ccc5ccccc5c4)cc2)cccc13 ; CC1(C)c2cc(N(c3ccc(-c4ccccc4)cc3)c3ccc(-c4ccccc4)cc3)ccc2-c2ccc3c(oc4ccccc43)c21 ; CC1(C)c2cc(N(c3ccccc3)c3ccc(-c4ccc5ccccc5c4)cc3)ccc2-c2ccc3c(oc4ccccc43)c21]

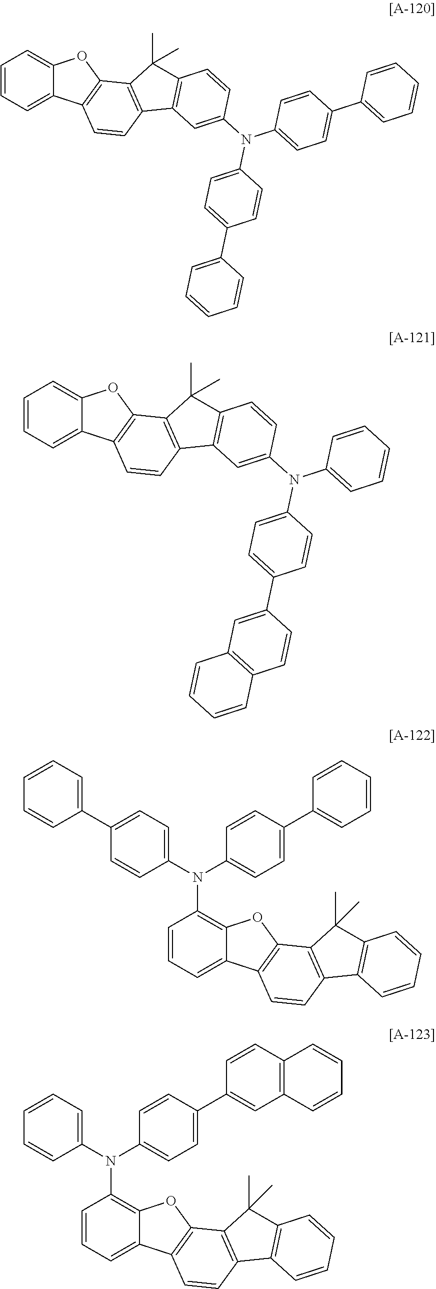

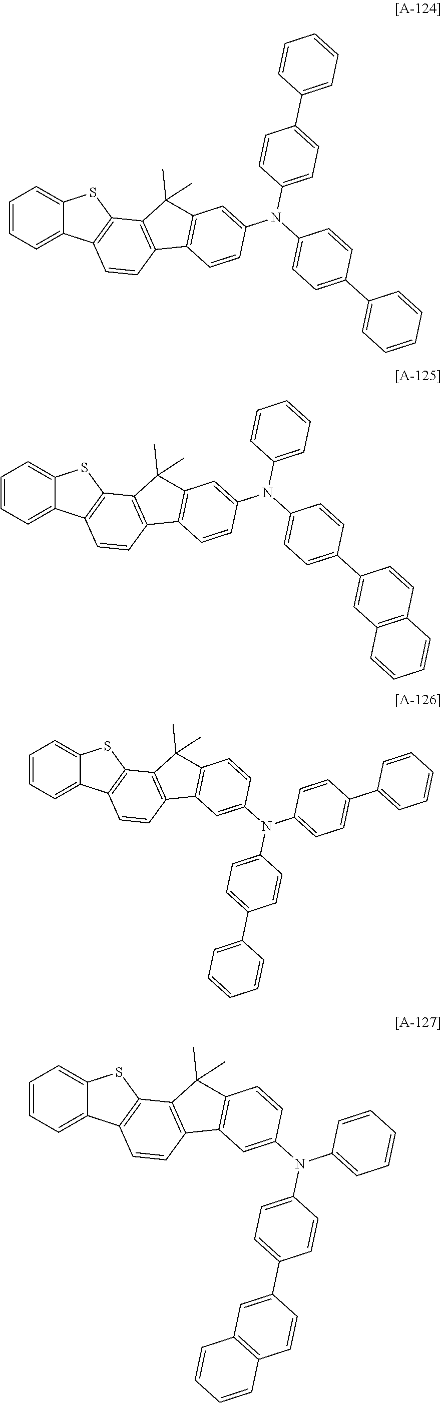 [SMILES: CC1(C)c2cc(N(c3ccc(-c4ccccc4)cc3)c3ccc(-c4ccccc4)cc3)ccc2-c2ccc3c(sc4ccccc43)c21 ; CC1(C)c2cc(N(c3ccccc3)c3ccc(-c4ccc5ccccc5c4)cc3)ccc2-c2ccc3c(sc4ccccc43)c21 ; CC1(C)c2ccc(N(c3ccc(-c4ccccc4)cc3)c3ccc(-c4ccccc4)cc3)cc2-c2ccc3c(sc4ccccc43)c21 ; CC1(C)c2ccc(N(c3ccccc3)c3ccc(-c4ccc5ccccc5c4)cc3)cc2-c2ccc3c(sc4ccccc43)c21]

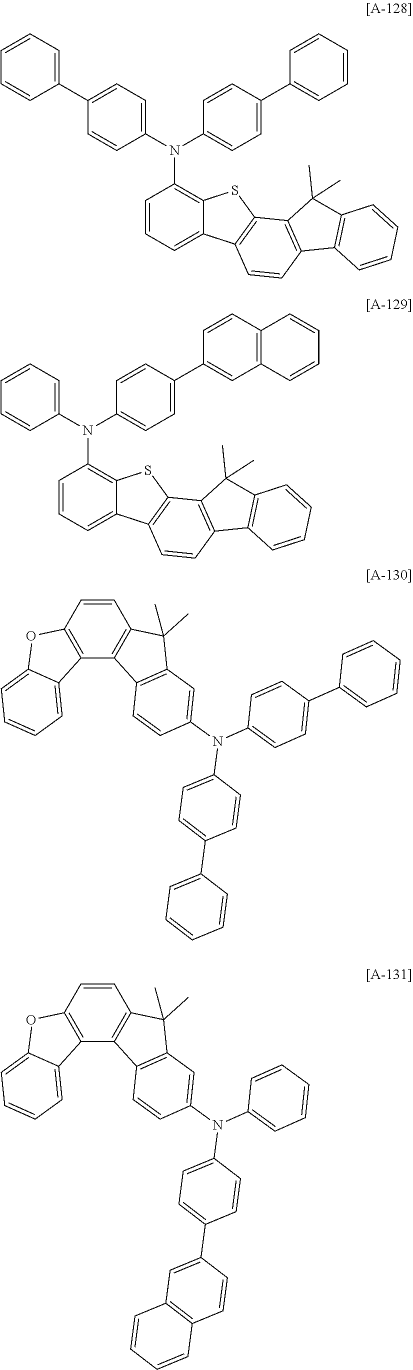

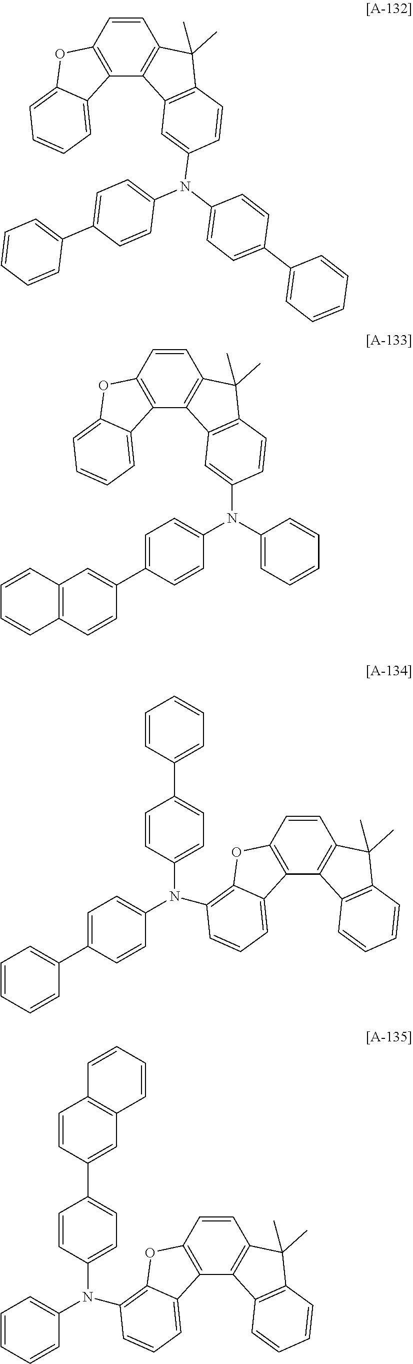 [SMILES: CC1(C)c2ccc(N(c3ccc(-c4ccccc4)cc3)c3ccc(-c4ccccc4)cc3)cc2-c2c1ccc1oc3ccccc3c21 ; CC1(C)c2ccc(N(c3ccccc3)c3ccc(-c4ccc5ccccc5c4)cc3)cc2-c2c1ccc1oc3ccccc3c21 ; CC1(C)c2ccccc2-c2c1ccc1oc3c(N(c4ccc(-c5ccccc5)cc4)c4ccc(-c5ccccc5)cc4)cccc3c21 ; CC1(C)c2ccccc2-c2c1ccc1oc3c(N(c4ccccc4)c4ccc(-c5ccc6ccccc6c5)cc4)cccc3c21]

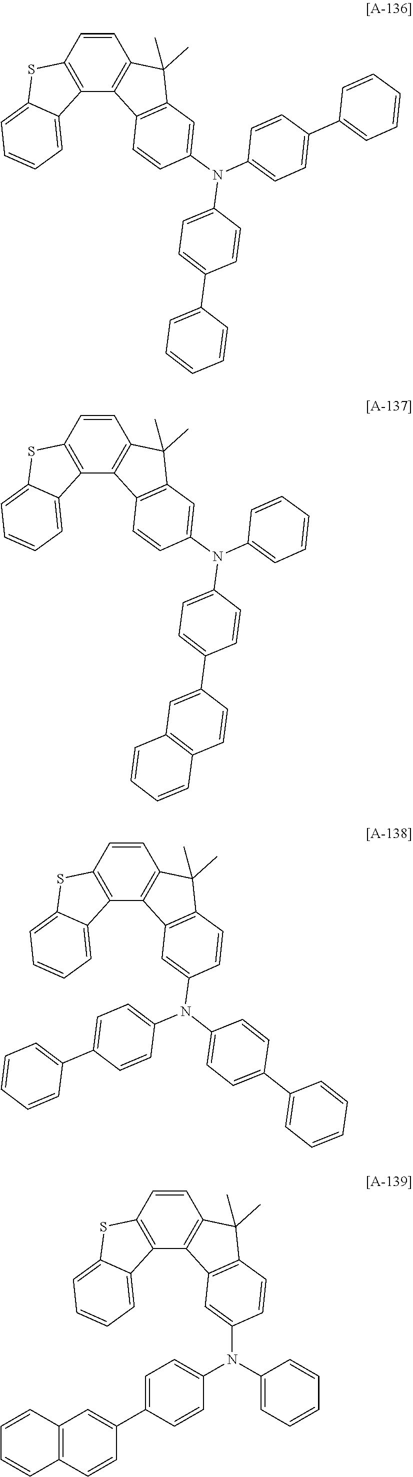

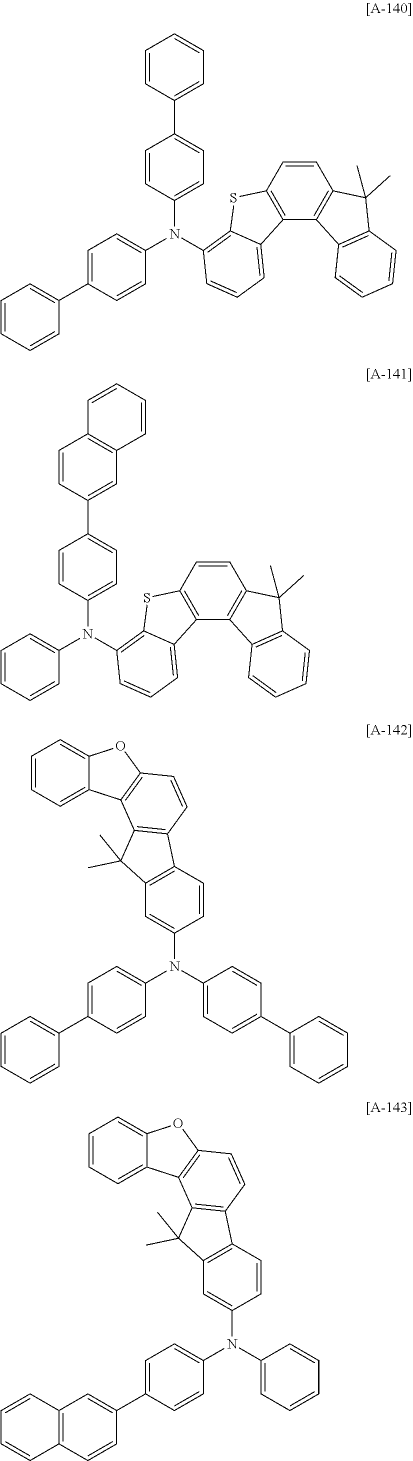 [SMILES: CC1(C)c2ccccc2-c2c1ccc1sc3c(N(c4ccc(-c5ccccc5)cc4)c4ccc(-c5ccccc5)cc4)cccc3c21 ; CC1(C)c2ccccc2-c2c1ccc1sc3c(N(c4ccccc4)c4ccc(-c5ccc6ccccc6c5)cc4)cccc3c21 ; CC1(C)c2cc(N(c3ccc(-c4ccccc4)cc3)c3ccc(-c4ccccc4)cc3)ccc2-c2ccc3oc4ccccc4c3c21 ; CC1(C)c2cc(N(c3ccccc3)c3ccc(-c4ccc5ccccc5c4)cc3)ccc2-c2ccc3oc4ccccc4c3c21]

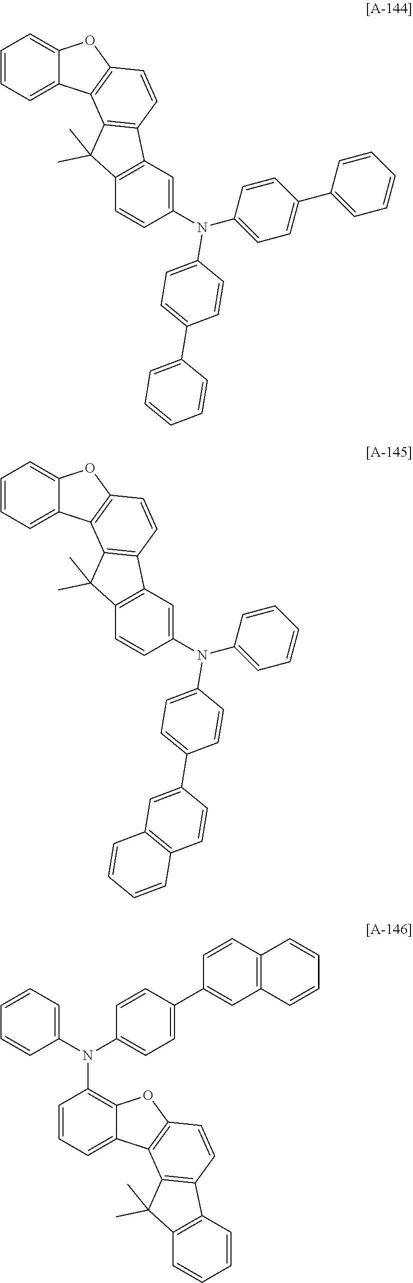 [SMILES: CC1(C)c2ccc(N(c3ccc(-c4ccccc4)cc3)c3ccc(-c4ccccc4)cc3)cc2-c2ccc3oc4ccccc4c3c21 ; CC1(C)c2ccc(N(c3ccccc3)c3ccc(-c4ccc5ccccc5c4)cc3)cc2-c2ccc3oc4ccccc4c3c21 ; CC1(C)c2ccccc2-c2ccc3oc4c(N(c5ccccc5)c5ccc(-c6ccc7ccccc7c6)cc5)cccc4c3c21]

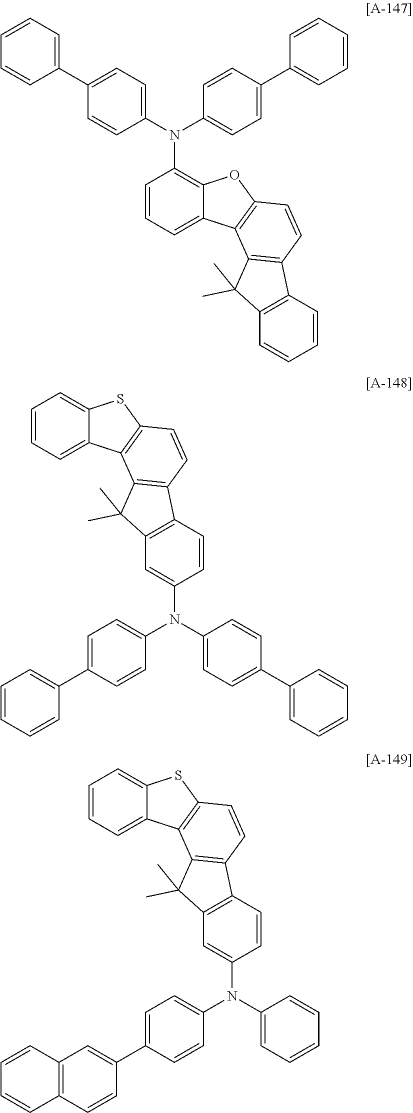 [SMILES: CC1(C)c2ccccc2-c2ccc3oc4c(N(c5ccc(-c6ccccc6)cc5)c5ccc(-c6ccccc6)cc5)cccc4c3c21 ; CC1(C)c2cc(N(c3ccc(-c4ccccc4)cc3)c3ccc(-c4ccccc4)cc3)ccc2-c2ccc3sc4ccccc4c3c21 ; CC1(C)c2cc(N(c3ccccc3)c3ccc(-c4ccc5ccccc5c4)cc3)ccc2-c2ccc3sc4ccccc4c3c21]

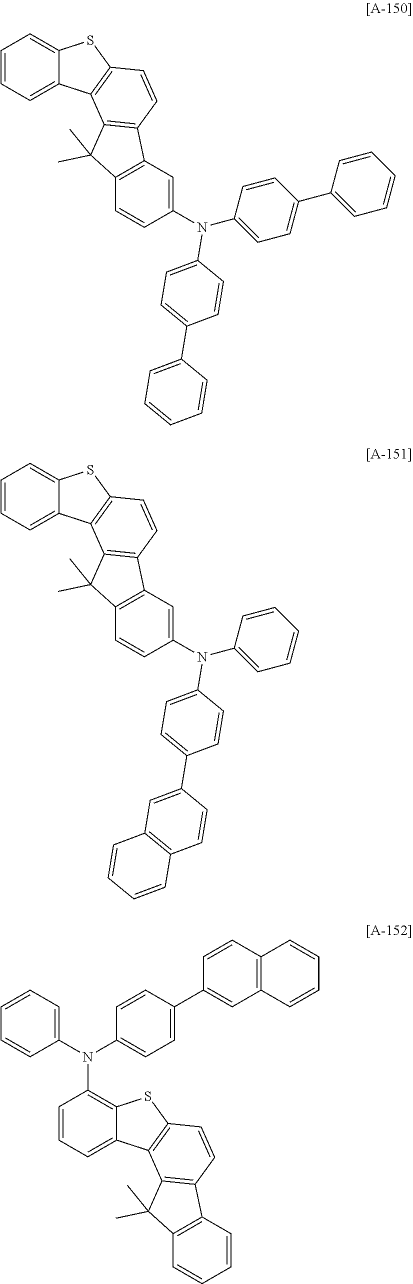 [SMILES: CC1(C)c2ccc(N(c3ccc(-c4ccccc4)cc3)c3ccc(-c4ccccc4)cc3)cc2-c2ccc3sc4ccccc4c3c21 ; CC1(C)c2ccc(N(c3ccccc3)c3ccc(-c4ccc5ccccc5c4)cc3)cc2-c2ccc3sc4ccccc4c3c21 ; CC1(C)c2ccccc2-c2ccc3sc4c(N(c5ccccc5)c5ccc(-c6ccc7ccccc7c6)cc5)cccc4c3c21]

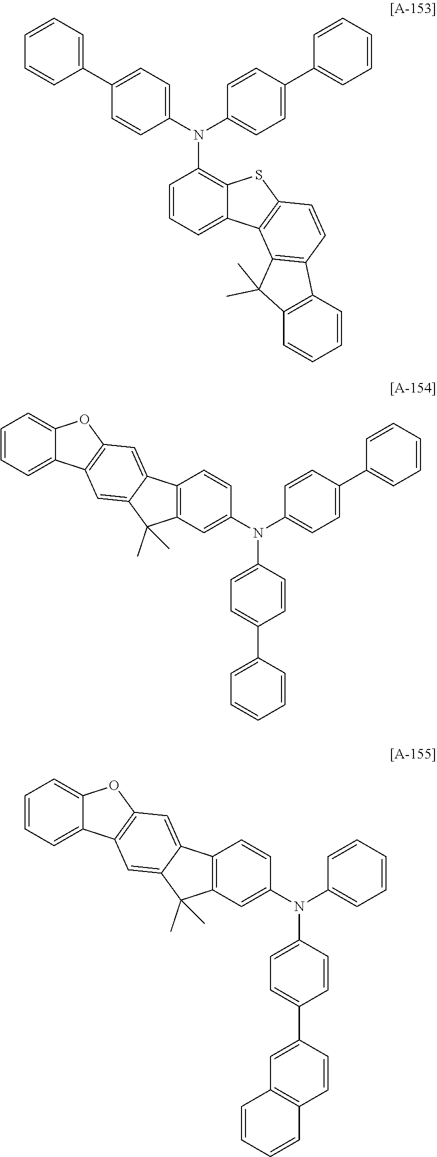 [SMILES: CC1(C)c2ccccc2-c2ccc3sc4c(N(c5ccc(-c6ccccc6)cc5)c5ccc(-c6ccccc6)cc5)cccc4c3c21 ; CC1(C)c2cc(N(c3ccc(-c4ccccc4)cc3)c3ccc(-c4ccccc4)cc3)ccc2-c2cc3oc4ccccc4c3cc21 ; CC1(C)c2cc(N(c3ccccc3)c3ccc(-c4ccc5ccccc5c4)cc3)ccc2-c2cc3oc4ccccc4c3cc21]

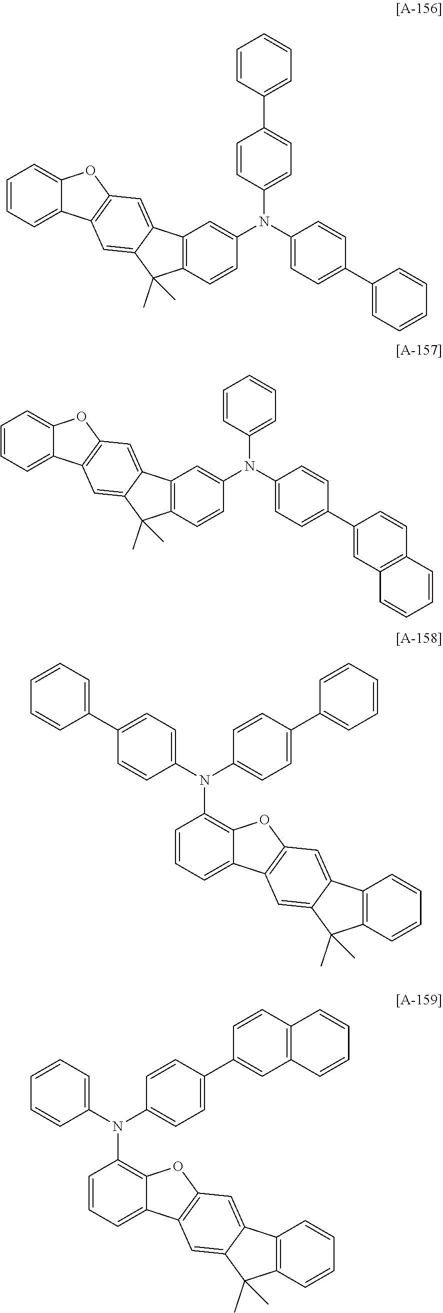

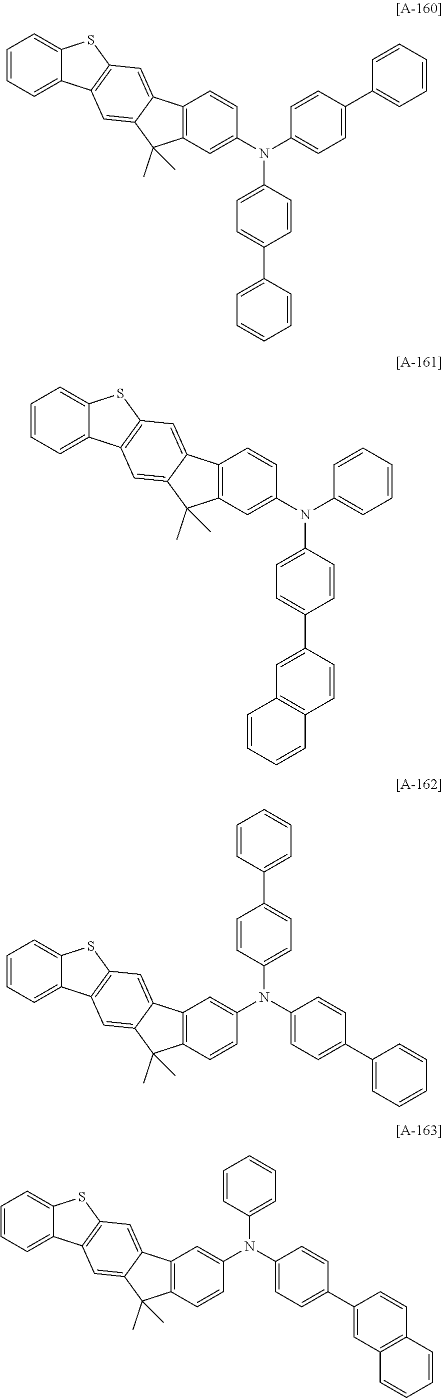 [SMILES: CC1(C)c2cc(N(c3ccc(-c4ccccc4)cc3)c3ccc(-c4ccccc4)cc3)ccc2-c2cc3sc4ccccc4c3cc21 ; CC1(C)c2cc(N(c3ccccc3)c3ccc(-c4ccc5ccccc5c4)cc3)ccc2-c2cc3sc4ccccc4c3cc21 ; CC1(C)c2ccc(N(c3ccc(-c4ccccc4)cc3)c3ccc(-c4ccccc4)cc3)cc2-c2cc3sc4ccccc4c3cc21 ; CC1(C)c2ccc(N(c3ccccc3)c3ccc(-c4ccc5ccccc5c4)cc3)cc2-c2cc3sc4ccccc4c3cc21]

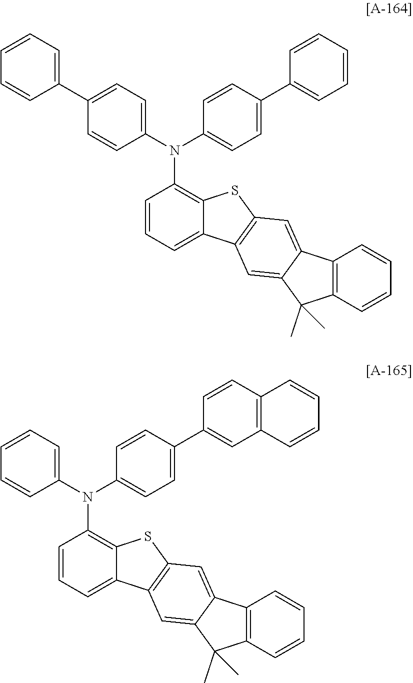

The second compound may be represented by Chemical Formula 3.

The second compound may be a compound capable of accepting electrons, when an electric field is applied, e.g., a compound having electron characteristics. In an implementation, the second compound may have a structure in which a triphenylene ring is linked with a nitrogen-containing ring, e.g., a pyrimidine or triazine ring to easily accept electrons when an electric field is applied. For example, a driving voltage of an organic optoelectronic device including the second compound may be lowered.

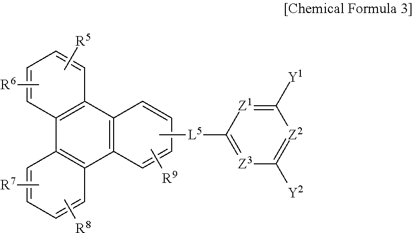

In Chemical Formula 3,

Z¹ to Z³ may each independently be, e.g., N or CR^{d}. In an implementation, at least two of Z¹ to Z³ may be N.

Y¹ and Y² may each independently be or include, e.g., hydrogen, deuterium, a substituted or unsubstituted C1 to C30 alkyl group, a substituted or unsubstituted C6 to C30 aryl group, a substituted or unsubstituted C2 to C30 heterocyclic group, a halogen, a cyano group, or a combination thereof.

L⁵ may be or may include, e.g., a single bond or a substituted or unsubstituted C6 to C20 arylene group.

R⁵ to R⁹ and R^{d} may each independently be or include, e.g., hydrogen, deuterium, a substituted or unsubstituted C1 to C30 alkyl group, a substituted or unsubstituted C6 to C30 aryl group, a halogen, a cyano group, or a combination thereof.

The second compound may exhibit good interface characteristics and hole and electron transport capability together with the aforementioned first compound. For example, a driving voltage of an organic optoelectronic device including the same may be lowered.

In an implementation, two of Z¹ to Z³ may be nitrogen (N) and the remaining one may be CR^{d}.

In an implementation, Z¹ and Z² may be nitrogen and Z³ may be CR^{d}.

In an implementation, Z² and Z³ may be nitrogen and Z¹ may be CR^{d}.

In an implementation, Z¹ and Z³ may be nitrogen and Z² may be CR^{d}.

In an implementation, Z¹ to Z³ may each independently be nitrogen (N).

In an implementation, Y¹ and Y² may each independently be or include, e.g., hydrogen, deuterium, a substituted or unsubstituted C6 to C30 aryl group, a substituted or unsubstituted pyridinyl group, a substituted or unsubstituted pyrimidinyl group, a substituted or unsubstituted triazinyl group, a substituted or unsubstituted dibenzofuranyl group, a substituted or unsubstituted dibenzothiophenyl group, or a combination thereof.

In an implementation, Y¹ and Y² may each independently be or include, e.g., a substituted or unsubstituted phenyl group, a substituted or unsubstituted biphenyl group, a substituted or unsubstituted terphenyl group, a substituted or unsubstituted naphthyl group, a substituted or unsubstituted triphenylenyl group, a substituted or unsubstituted pyridinyl group, a substituted or unsubstituted pyrimidinyl group, a substituted or unsubstituted triazinyl group, a substituted or unsubstituted dibenzofuranyl group, or a substituted or unsubstituted dibenzothiophenyl group. Here, “substituted” may refer to, e.g., replacement of at least one hydrogen by deuterium, a C1 to C20 alkyl group, a C6 to C20 aryl group, a pyridinyl group, a pyrimidinyl group, a triazinyl group, a halogen, a cyano group, or a combination thereof.

In an implementation, L⁵ may be or may include, e.g., a single bond, a substituted or unsubstituted phenylene group, a substituted or unsubstituted biphenylene group, or a substituted or unsubstituted terphenylene group.

In an implementation, L⁵ may be or may include, e.g., a single bond, a substituted or unsubstituted m-phenylene group, a substituted or unsubstituted p-phenylene group, a substituted or unsubstituted o-phenylene group, a substituted or unsubstituted m-biphenylene group, a substituted or unsubstituted p-biphenylene group, a substituted or unsubstituted o-biphenylene group, a substituted or unsubstituted m-terphenylene group, a substituted or unsubstituted p-terphenylene group, or a substituted or unsubstituted o-terphenylene group. Herein, “substituted” may for example refer to replacement of at least one hydrogen by deuterium, a C1 to C20 alkyl group, a C6 to C20 aryl group, a halogen, a cyano group, or a combination thereof.

In an implementation, L⁵ may be, e.g., a single bond, an unsubstituted phenylene group, an unsubstituted biphenylene group, an unsubstituted terphenylene group, a phenylene group substituted with a phenyl group or a cyano group, a biphenylene group substituted with a phenyl group or a cyano group, or a terphenylene group substituted with a phenyl group or a cyano group.

In an implementation, L⁵ may be, e.g., a single bond or a linking group of Group I.

### [Group I]

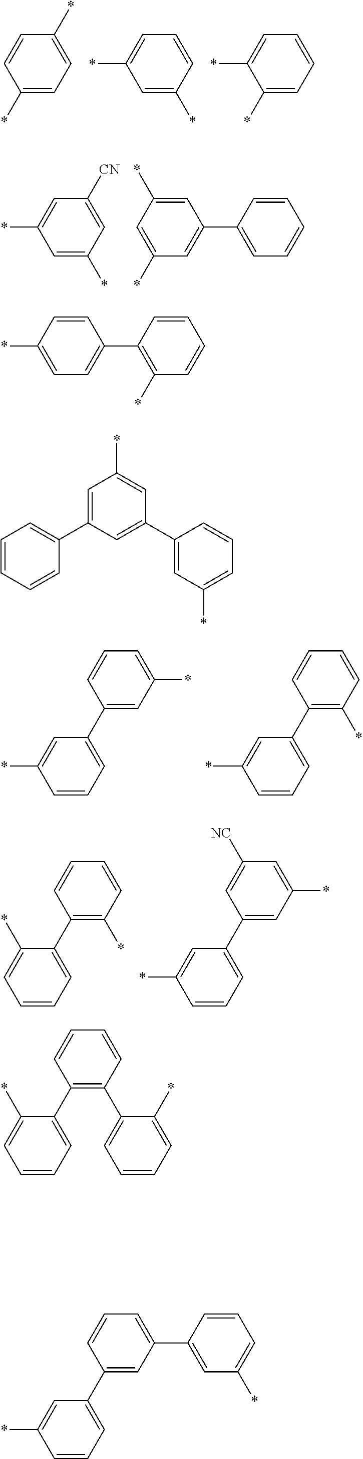

In an implementation, the moiety

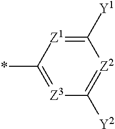

of Chemical Formula 3 may be a moiety of Group II.

### [Group II]

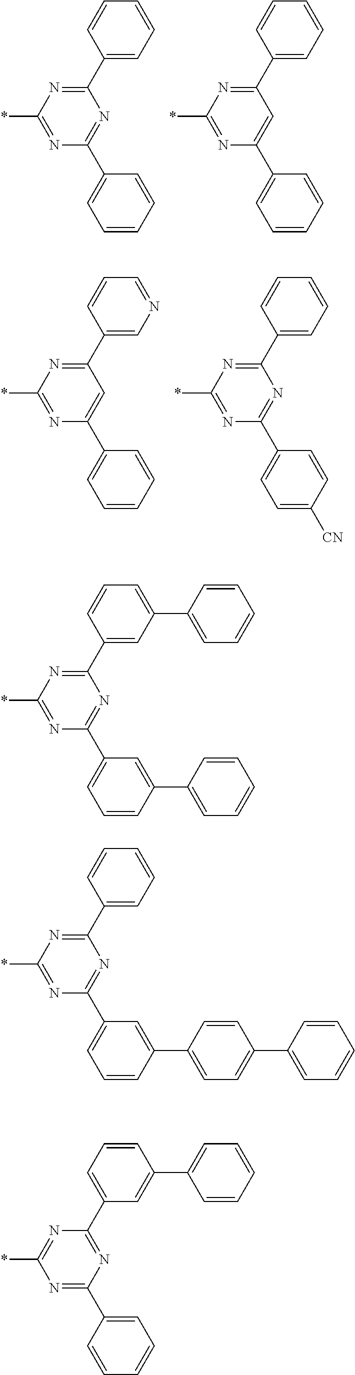

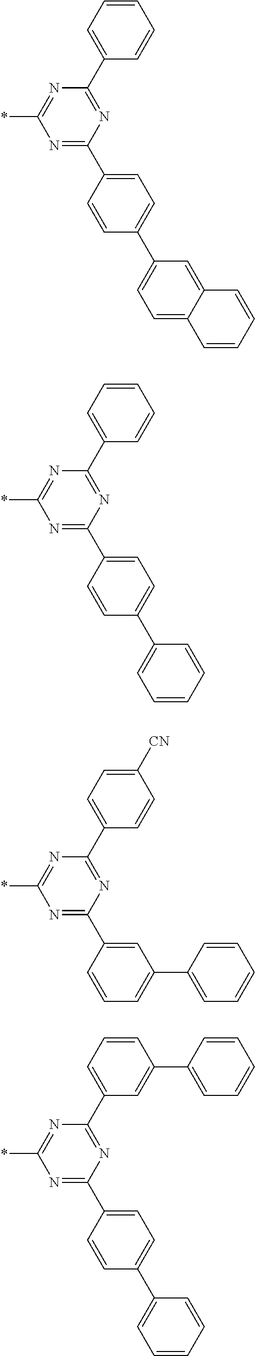

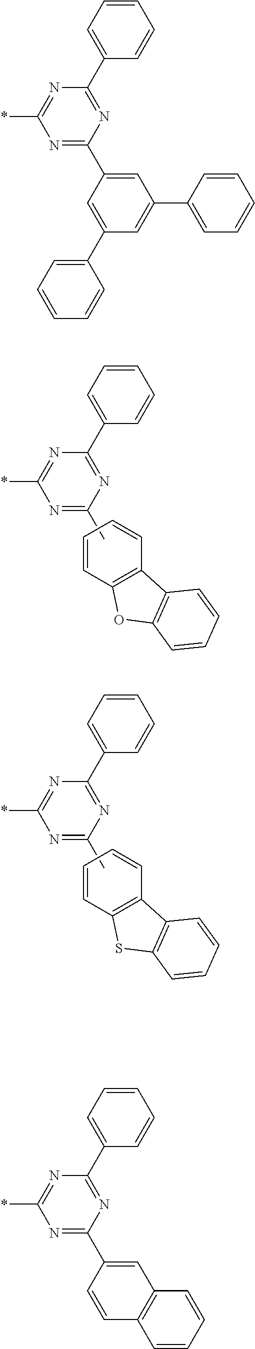

In an implementation, the second compound represented by Chemical Formula 3 may be represented by Chemical Formula 3A or Chemical Formula 3B (e.g., according to a linking position of the triphenylene ring with the pyrimidine or triazine ring).

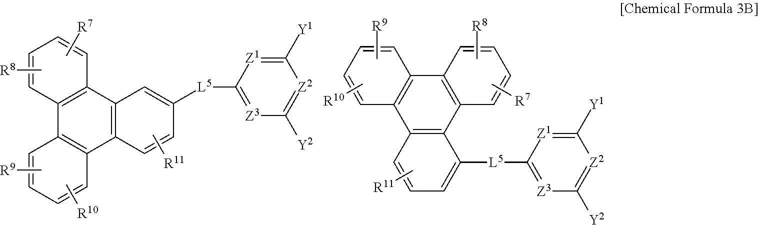

In Chemical Formulae 3A and 3B, Z¹ to Z³, Y¹, Y², L⁵, and R⁷ to R¹¹ may be defined the same as those of Chemical Formula 3.

In an implementation, the second compound may be represented by Chemical Formula 3A.

In an implementation, the second compound may be represented by Chemical Formula 3A-1.

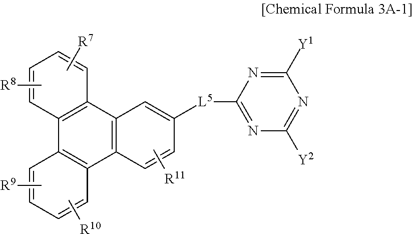

In Chemical Formula 3A-1, Y¹ and Y², L⁵, and R⁷ to R¹¹ may be defined the same as those of Chemical Formula 3.

In an implementation, the second compound may be, e.g., a compound Group 2.

### [Group 2]

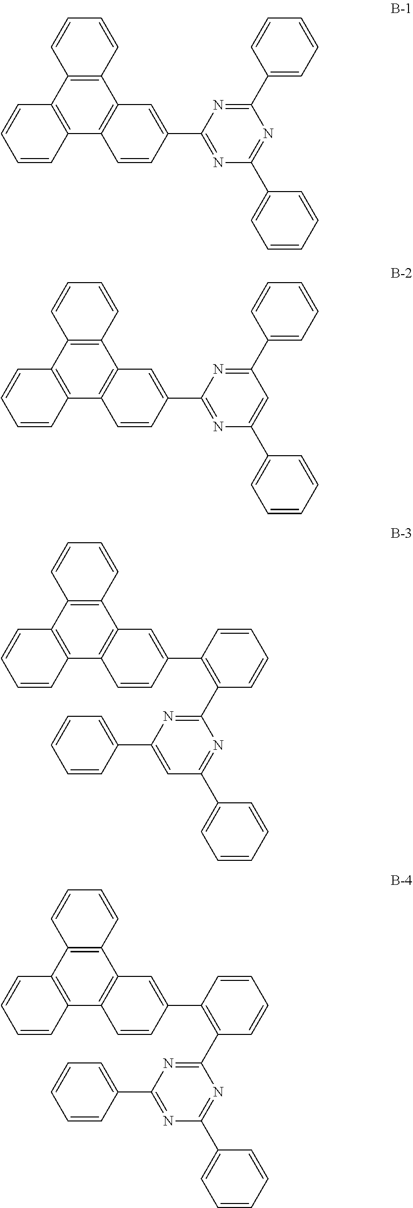

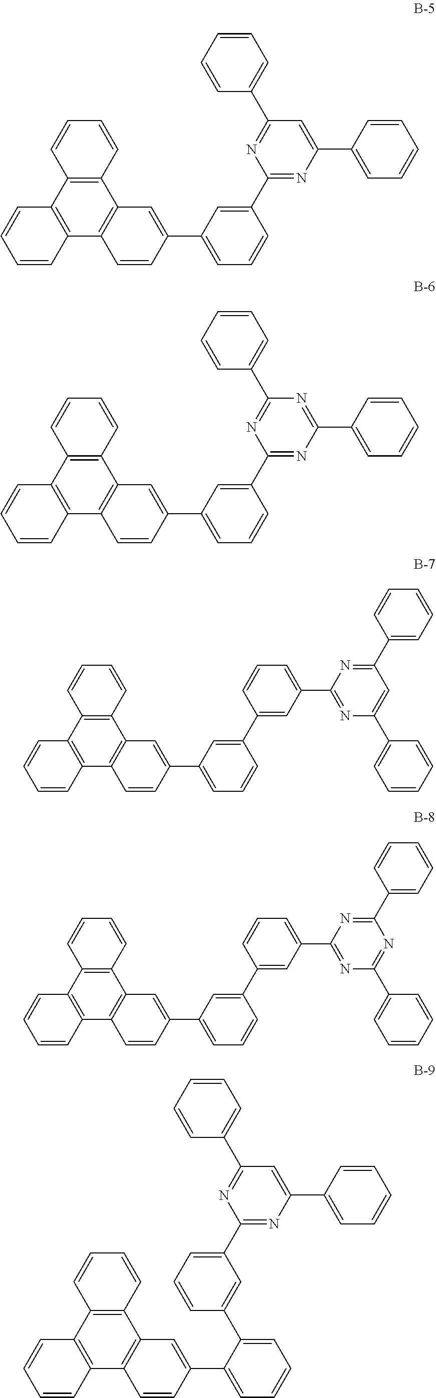

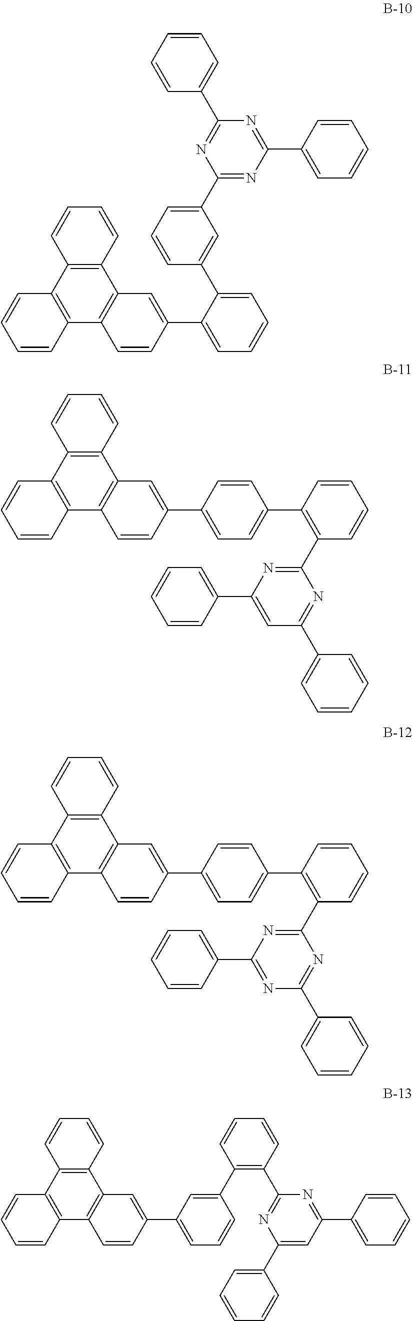

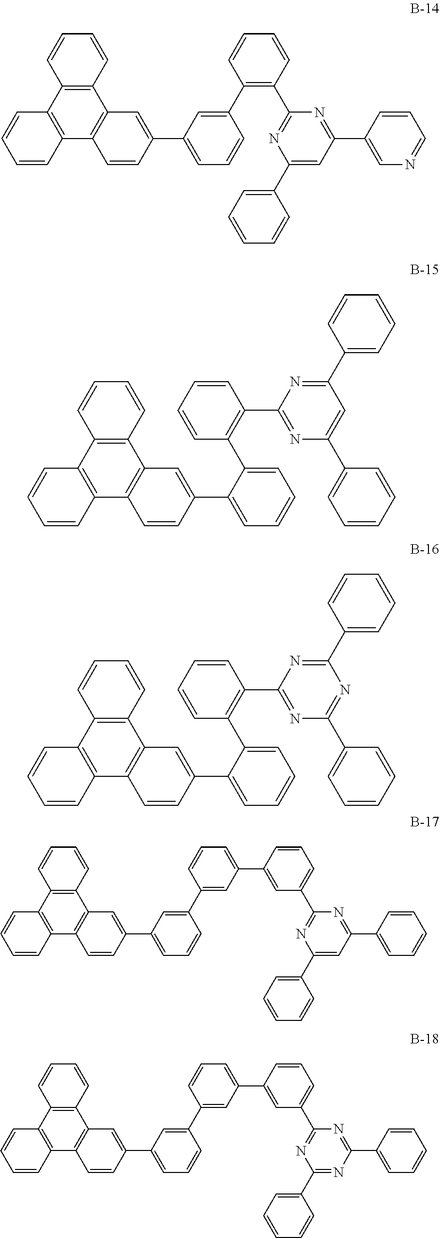

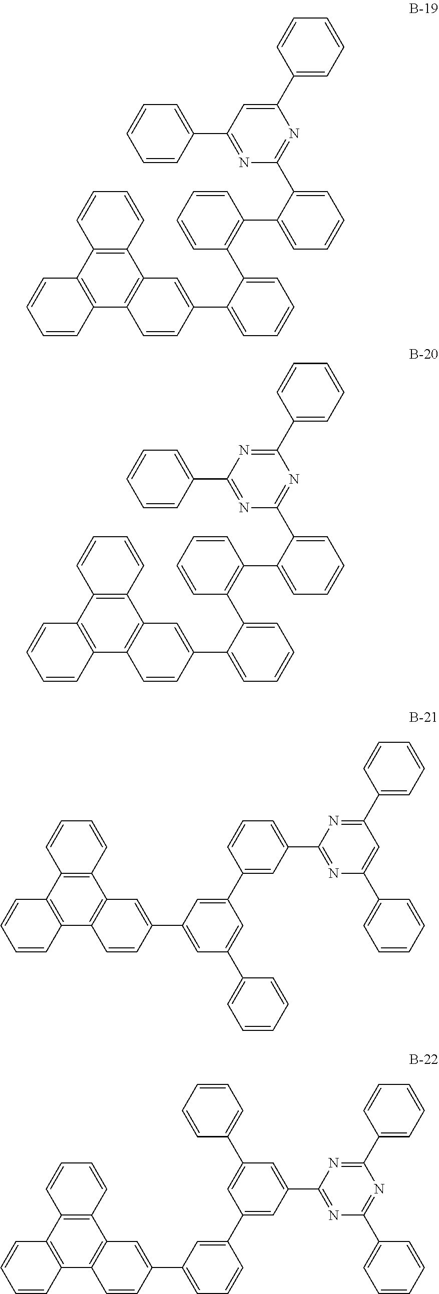

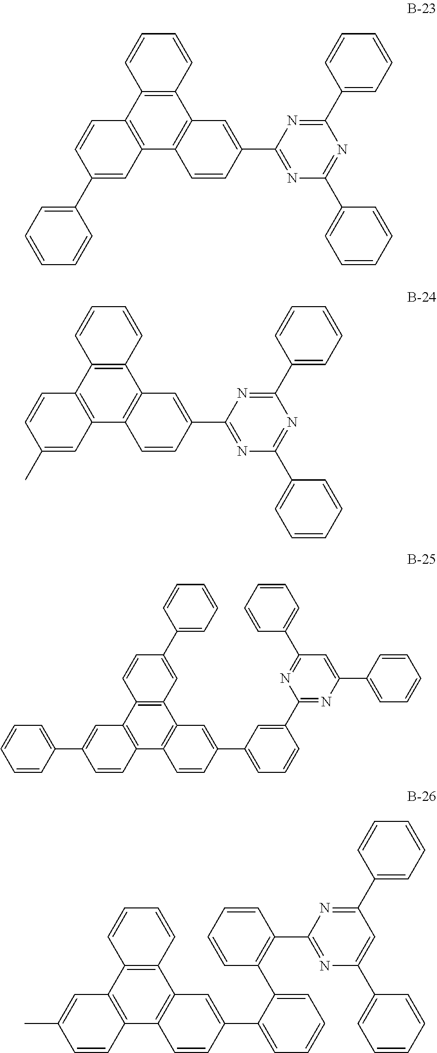

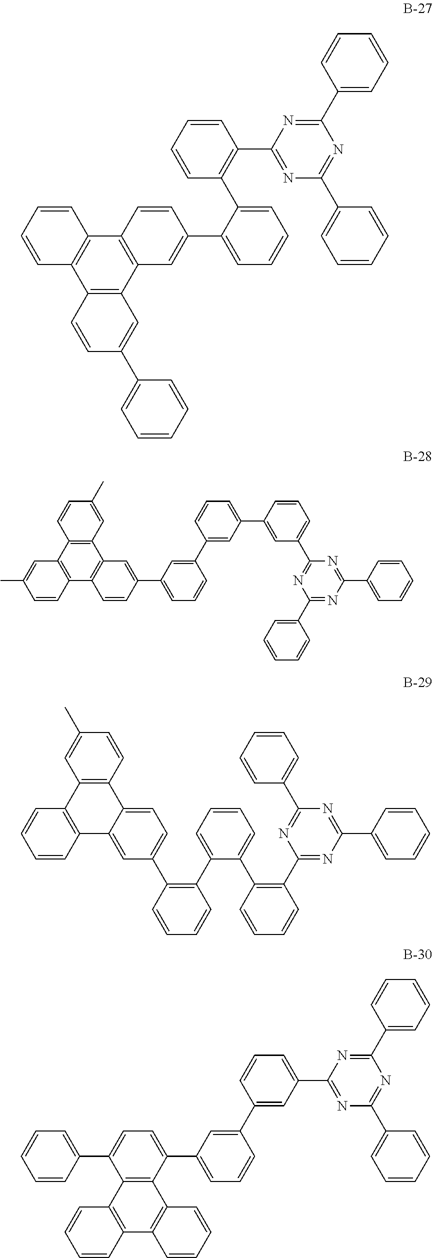 [SMILES: c1ccc(-c2ccc3c4ccccc4c4ccc(-c5ccccc5-c5ccccc5-c5nc(-c6ccccc6)nc(-c6ccccc6)n5)cc4c3c2)cc1 ; Cc1ccc2c3ccc(-c4cccc(-c5cccc(-c6cccc(-c7nc(-c8ccccc8)nc(-c8ccccc8)n7)c6)c5)c4)cc3c3cc(C)ccc3c2c1 ; Cc1ccc2c3ccc(-c4ccccc4-c4ccccc4-c4ccccc4-c4nc(-c5ccccc5)nc(-c5ccccc5)n4)cc3c3ccccc3c2c1 ; c1ccc(-c2nc(-c3ccccc3)nc(-c3cccc(-c4cccc(-c5ccc(-c6ccccc6)c6c7ccccc7c7ccccc7c56)c4)c3)n2)cc1]

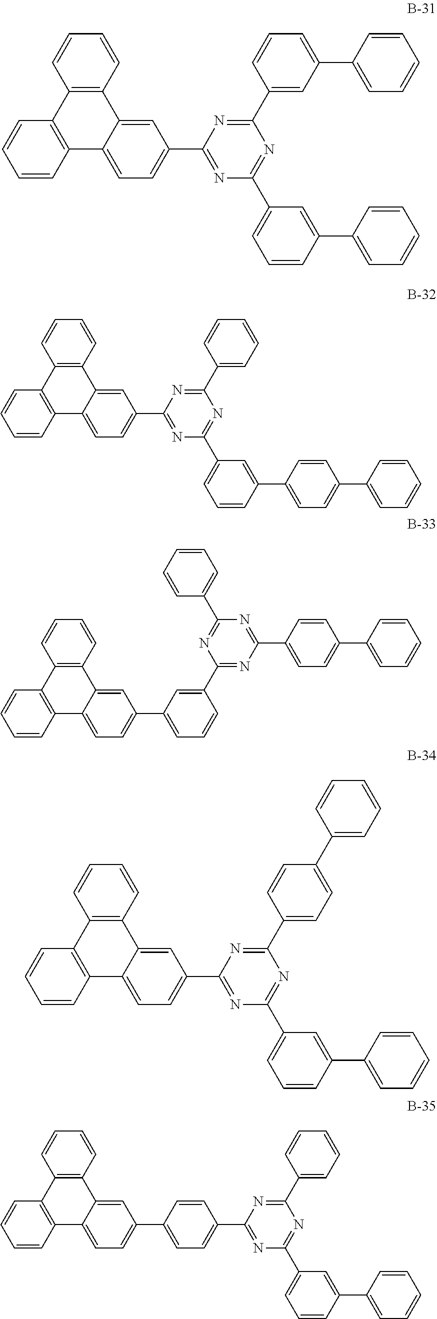 [SMILES: c1ccc(-c2cccc(-c3nc(-c4cccc(-c5ccccc5)c4)nc(-c4ccc5c6ccccc6c6ccccc6c5c4)n3)c2)cc1 ; c1ccc(-c2ccc(-c3cccc(-c4nc(-c5ccccc5)nc(-c5ccc6c7ccccc7c7ccccc7c6c5)n4)c3)cc2)cc1 ; c1ccc(-c2ccc(-c3nc(-c4ccccc4)nc(-c4cccc(-c5ccc6c7ccccc7c7ccccc7c6c5)c4)n3)cc2)cc1 ; c1ccc(-c2ccc(-c3nc(-c4cccc(-c5ccccc5)c4)nc(-c4ccc5c6ccccc6c6ccccc6c5c4)n3)cc2)cc1 ; c1ccc(-c2cccc(-c3nc(-c4ccccc4)nc(-c4ccc(-c5ccc6c7ccccc7c7ccccc7c6c5)cc4)n3)c2)cc1]

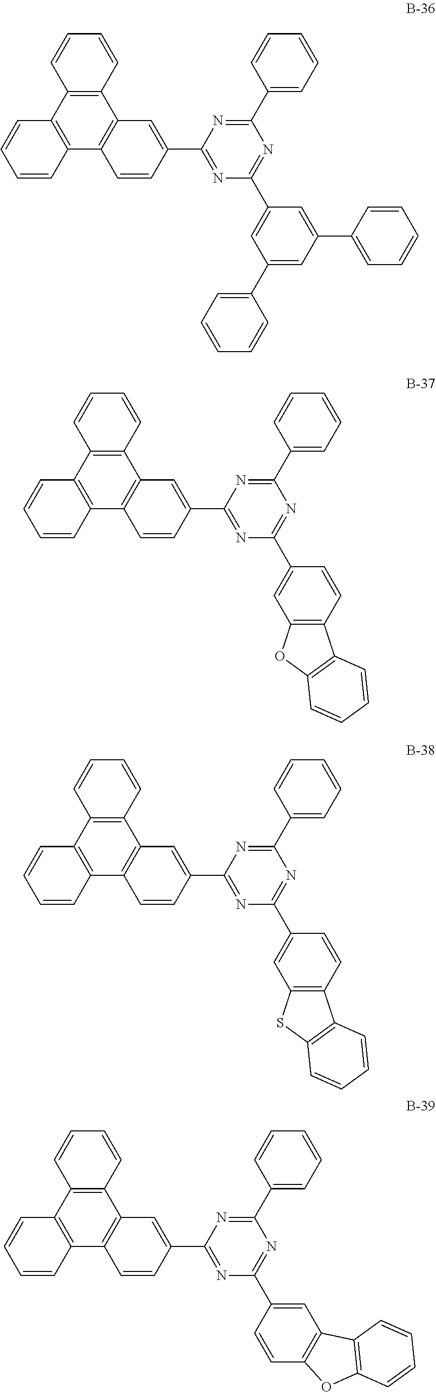

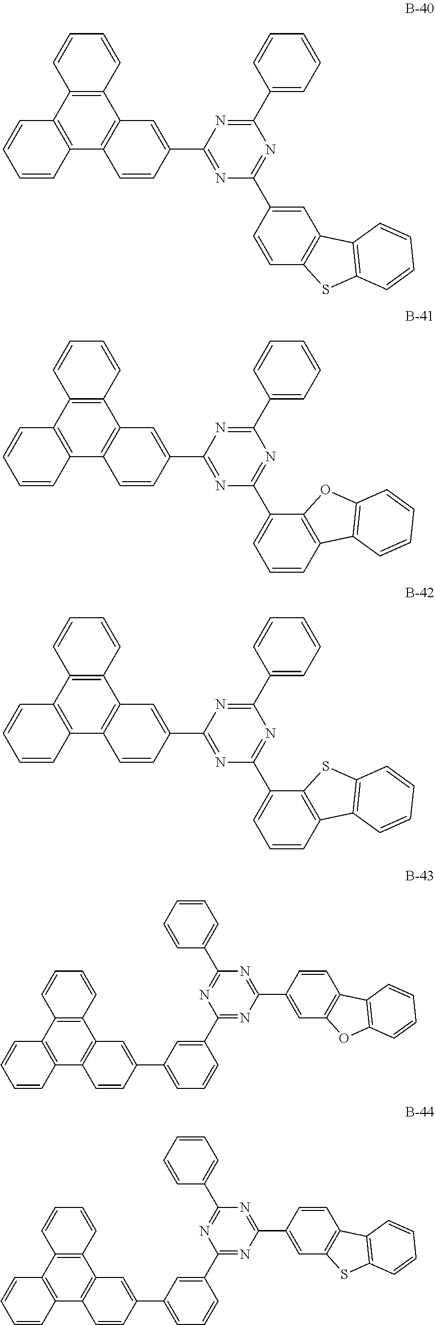

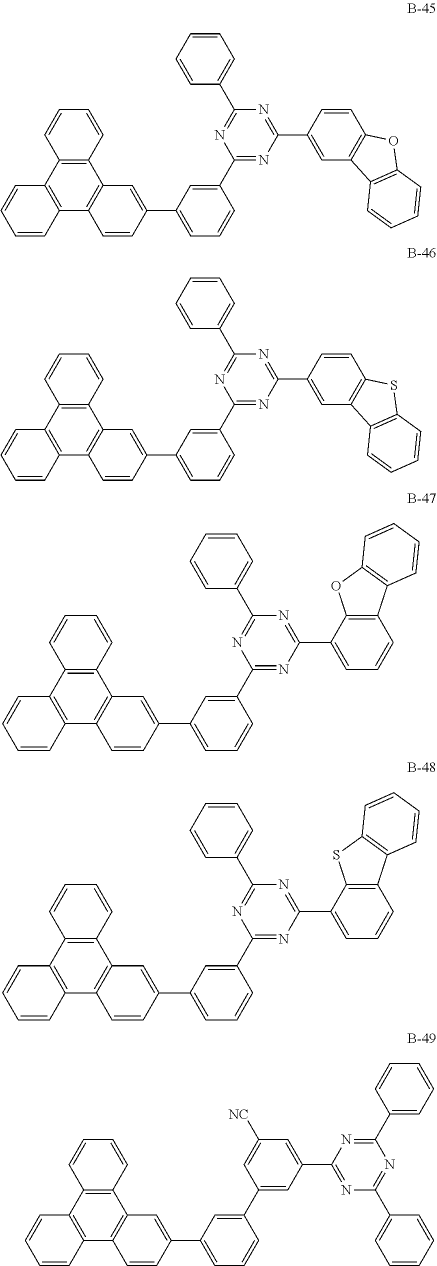 [SMILES: c1ccc(-c2nc(-c3cccc(-c4ccc5c6ccccc6c6ccccc6c5c4)c3)nc(-c3ccc4oc5ccccc5c4c3)n2)cc1 ; c1ccc(-c2nc(-c3cccc(-c4ccc5c6ccccc6c6ccccc6c5c4)c3)nc(-c3ccc4sc5ccccc5c4c3)n2)cc1 ; c1ccc(-c2nc(-c3cccc(-c4ccc5c6ccccc6c6ccccc6c5c4)c3)nc(-c3cccc4c3oc3ccccc34)n2)cc1 ; c1ccc(-c2nc(-c3cccc(-c4ccc5c6ccccc6c6ccccc6c5c4)c3)nc(-c3cccc4c3sc3ccccc34)n2)cc1 ; N#Cc1cc(-c2cccc(-c3ccc4c5ccccc5c5ccccc5c4c3)c2)cc(-c2nc(-c3ccccc3)nc(-c3ccccc3)n2)c1]

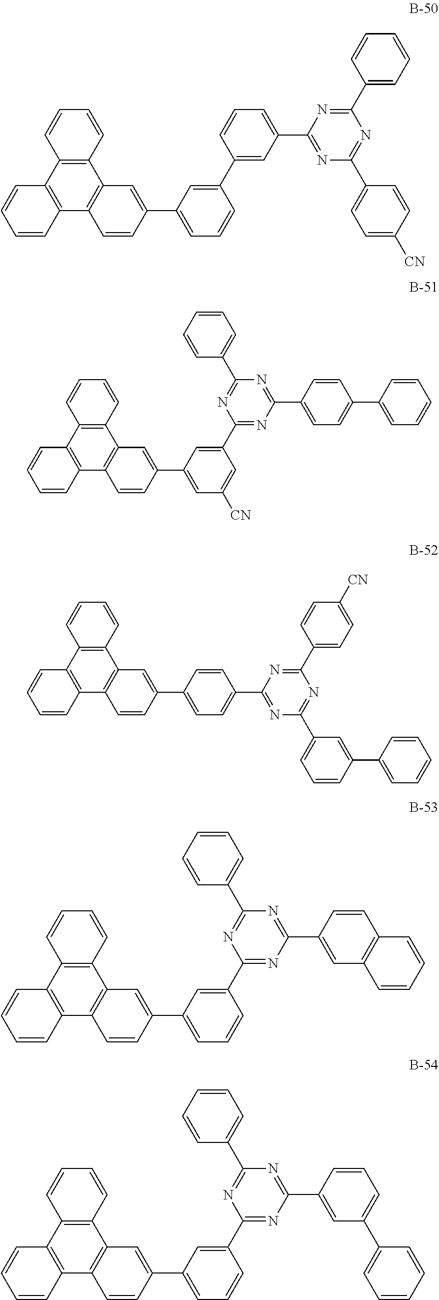 [SMILES: N#Cc1ccc(-c2nc(-c3ccccc3)nc(-c3cccc(-c4cccc(-c5ccc6c7ccccc7c7ccccc7c6c5)c4)c3)n2)cc1 ; N#Cc1cc(-c2ccc3c4ccccc4c4ccccc4c3c2)cc(-c2nc(-c3ccccc3)nc(-c3ccc(-c4ccccc4)cc3)n2)c1 ; N#Cc1ccc(-c2nc(-c3ccc(-c4ccc5c6ccccc6c6ccccc6c5c4)cc3)nc(-c3cccc(-c4ccccc4)c3)n2)cc1 ; c1ccc(-c2nc(-c3cccc(-c4ccc5c6ccccc6c6ccccc6c5c4)c3)nc(-c3ccc4ccccc4c3)n2)cc1 ; c1ccc(-c2cccc(-c3nc(-c4ccccc4)nc(-c4cccc(-c5ccc6c7ccccc7c7ccccc7c6c5)c4)n3)c2)cc1]

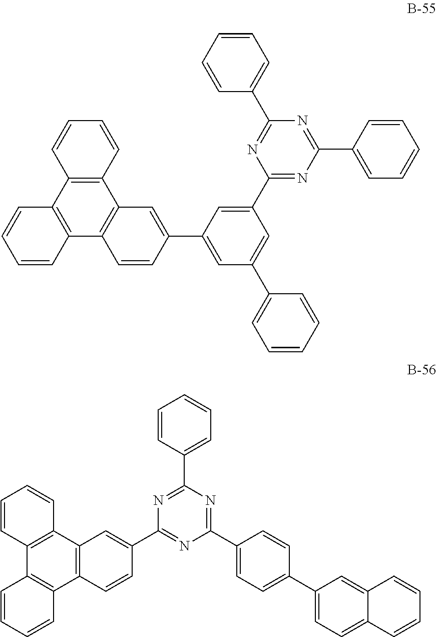 [SMILES: c1ccc(-c2cc(-c3ccc4c5ccccc5c5ccccc5c4c3)cc(-c3nc(-c4ccccc4)nc(-c4ccccc4)n3)c2)cc1 ; c1ccc(-c2nc(-c3ccc(-c4ccc5ccccc5c4)cc3)nc(-c3ccc4c5ccccc5c5ccccc5c4c3)n2)cc1]

The first compound and the second compound may be included in the composition in a weight ratio of, e.g., about 1:99 to about 99:1. Within the range, a desirable weight ratio may be adjusted using a hole transport capability of the first compound and an electron transport capability of the second compound for the organic optoelectronic device to realize bipolar characteristics and thus to improve efficiency and life-span. Within the range, they may be, e.g., included in a weight ratio of about 90:10 to about 10:90, about 80:20 to about 20:80 or about 70:30 to about 30:70. In an implementation, they may be included in a weight ratio of, e.g., about 70:30 to about 40:60 or about 70:30 to about 50:50. In an implementation, they may be included in a weight ratio of, e.g., about 70:30, about 60:40, or about 50:50.

In an implementation, the composition for the organic optoelectronic device may include, e.g., the compound represented by Chemical Formula 1E-2-2 as the first compound and the compound represented by Chemical Formula 3A-1 as the second compound.

In an implementation, in Chemical Formula 1E-2-2, L^{a}, L^{b}, L^{c}, and L¹ to L⁴ may each independently be or include, e.g., a single bond, a substituted or unsubstituted phenylene group, a substituted or unsubstituted biphenylene group, a substituted or unsubstituted terphenylene group, or a substituted or unsubstituted naphthylene group, R^{a}, R¹, R², and R⁴ may each independently be or include, e.g., hydrogen, deuterium, a cyano group, a substituted or unsubstituted C1 to C30 alkyl group, a substituted or unsubstituted C6 to C30 aryl group, a substituted or unsubstituted C2 to C30 heterocyclic group, or a combination thereof, R^{b} and R^{c} may each independently be or include, e.g., a substituted or unsubstituted phenyl group, a substituted or unsubstituted biphenyl group, a substituted or unsubstituted naphthyl group, a substituted or unsubstituted fluorenyl group, a substituted or unsubstituted carbazolyl group, a substituted or unsubstituted dibenzofuranyl group, a substituted or unsubstituted dibenzothiophenyl group, or a fused ring represented by a combination of Chemical Formula 1 and Chemical Formula 2.

In an implementation, in Chemical Formula 3A-1, Y¹ and Y² may each independently be or include, e.g., a substituted or unsubstituted phenyl group, a substituted or unsubstituted biphenyl group, a substituted or unsubstituted terphenyl group, a substituted or unsubstituted dibenzofuranyl group, a substituted or unsubstituted dibenzothiophenyl group, L⁵ may be, e.g., a single bond or a (e.g., substituted or unsubstituted) phenylene group, and R⁷ to R¹¹ may each independently be or include, e.g., hydrogen, deuterium, a substituted or unsubstituted C1 to C10 alkyl group, a substituted or unsubstituted C6 to C12 aryl group, a cyano group, or a combination thereof.

In an implementation, L^{a} and L¹ to L⁴ of Chemical Formula 1E-2-2 may each be, e.g., a single bond, and L^{b} and L^{c} may each independently be or include, e.g., a single bond or a substituted or unsubstituted C6 to C18 arylene group. In an implementation, at least one of L^{b} and L^{c} may be, e.g., a substituted or unsubstituted C6 to C18 arylene group,

In an implementation, R^{a}, R¹, R², and R⁴ may each be, e.g., hydrogen, R⁵ and R⁶ may each independently be or include, e.g., a substituted or unsubstituted C1 to C10 alkyl group or a substituted or unsubstituted C6 to C20 aryl group, and R^{b} and R^{c} may each independently be or include, e.g., a substituted or unsubstituted phenyl group, a substituted or unsubstituted biphenyl group, a substituted or unsubstituted naphthyl group, or a substituted or unsubstituted fluorenyl group.

In an implementation, Y¹ and Y² of Chemical Formula 3A-1 may each independently be or include, e.g., a substituted or unsubstituted phenyl group, a substituted or unsubstituted biphenyl group or a substituted or unsubstituted naphthyl group, L⁵ may be, e.g., a single bond or a phenylene group, and R⁷ to R¹¹ may each be, e.g., hydrogen.

In an implementation, the composition for the organic optoelectronic device may further include one or more compounds in addition to the aforementioned first compound and second compound.

In an implementation, the composition for the organic optoelectronic device may further include a dopant. The dopant may be, e.g., a phosphorescent dopant. In an implementation, the dopant may be, e.g., a red, green, or blue phosphorescent dopant. In an implementation, the dopant may be, e.g., a red phosphorescent dopant.

The dopant may be mixed with the first compound and the second compound in a small amount to cause light emission, and may include a material such as a metal complex that emits light by multiple excitation into a triplet or more. The dopant may be, e.g., an inorganic, organic, or organic/inorganic compound, and one or more types thereof may be used.

In an implementation, the dopant may be, e.g., a phosphorescent dopant. Examples of the phosphorescent dopant may include an organometal compound including Ir, Pt, Os, Ti, Zr, Hf, Eu, Tb, Tm, Fe, Co, Ni, Ru, Rh, Pd, or a combination thereof. In an implementation, the phosphorescent dopant may include, e.g., a compound represented by Chemical Formula Z.

L⁸MX⁵ [Chemical Formula Z]

In Chemical Formula Z, M may be, e.g., a metal, and L⁸ and X⁵ may each independently be, e.g., a ligand to form a complex compound with M.

In an implementation, M may be, e.g., Ir, Pt, Os, Ti, Zr, Hf, Eu, Tb, Tm, Fe, Co, Ni, Ru, Rh, Pd, or a combination thereof, and L⁸ and X⁴ may be, e.g., a bidendate ligand.

The composition for an organic optoelectronic device may be formed into a film using a dry film-forming method such as chemical vapor deposition.

Hereinafter, an organic optoelectronic device to which the aforementioned composition for the organic optoelectronic device is applied is described.

In an implementation, the organic optoelectronic device may be a suitable device to convert electrical energy into photoenergy and vice versa, and may be, e.g., an organic photoelectric device, an organic light emitting diode, an organic solar cell, and an organic photo-conductor drum.

Herein, an organic light emitting diode as one example of an organic optoelectronic device is described referring to drawings.

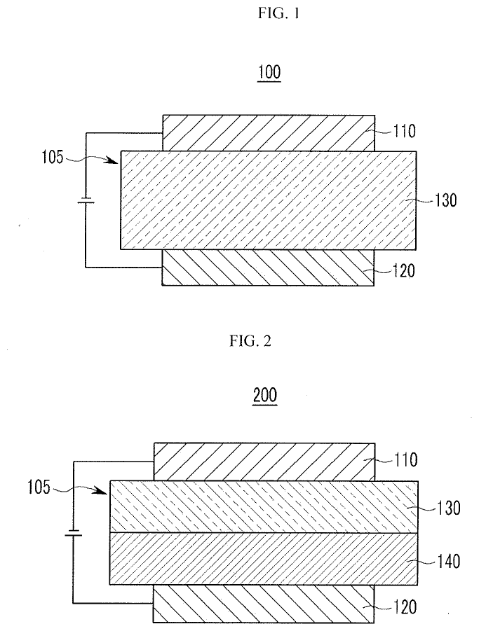

FIGS. 1 and 2 illustrate cross-sectional views of organic light emitting diodes according to embodiments.

Referring to FIG. 1, an organic light emitting diode 100 according to one embodiment may include an anode 120 and a cathode 110 facing each other and an organic layer 105 between the anode 120 and cathode 110.

The anode 120 may be made of a conductor having a large work function to facilitate hole injection, and may be e.g., a metal, a metal oxide, and/or a conductive polymer. In an implementation, the anode 120 may be, e.g., a metal such as nickel, platinum, vanadium, chromium, copper, zinc, gold, and the like or an alloy thereof; a metal oxide such as zinc oxide, indium oxide, indium tin oxide (ITO), indium zinc oxide (IZO), and the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, and polyaniline.

The cathode 110 may be made of a conductor having a small work function to facilitate electron injection, and may be, e.g., a metal, a metal oxide and/or a conductive polymer. In an implementation, the cathode 110 may be, e.g., a metal such as magnesium, calcium, sodium, potassium, titanium, indium, yttrium, lithium, gadolinium, aluminum silver, tin, lead, cesium, barium, and the like, or an alloy thereof; a multi-layer structure material such as LiF/Al, LiO₂/Al, LiF/Ca, LiF/Al, and BaF₂/Ca.

The organic layer 105 may include a light emitting layer 130 including the aforementioned composition for the organic optoelectronic device.

The composition for the organic optoelectronic device according to an embodiment may be, e.g., a red light emitting composition.

The light emitting layer 130 may include e.g., the aforementioned first compound and the aforementioned second compound as phosphorescent hosts.

Referring to FIG. 2, an organic light emitting diode 200 may further include a hole auxiliary layer 140 as well as a light emitting layer 130. The hole auxiliary layer 140 may further increase hole injection and/or hole mobility between the anode 120 and light emitting layer 130 and may help block electrons. The hole auxiliary layer 140 may be, e.g., a hole transport layer (HTL), a hole injection layer (HIL), and/or an electron blocking layer, and may include at least one layer.

The hole auxiliary layer 140 may include, e.g., a compound of Group E, below.

For example, the hole auxiliary layer 140 may include a hole transport layer between the anode 120 and the light emitting layer 130 and a hole transport auxiliary layer between the light emitting layer 130 and the hole transport layer, and a compound of Group E may be included in the hole transport auxiliary layer.

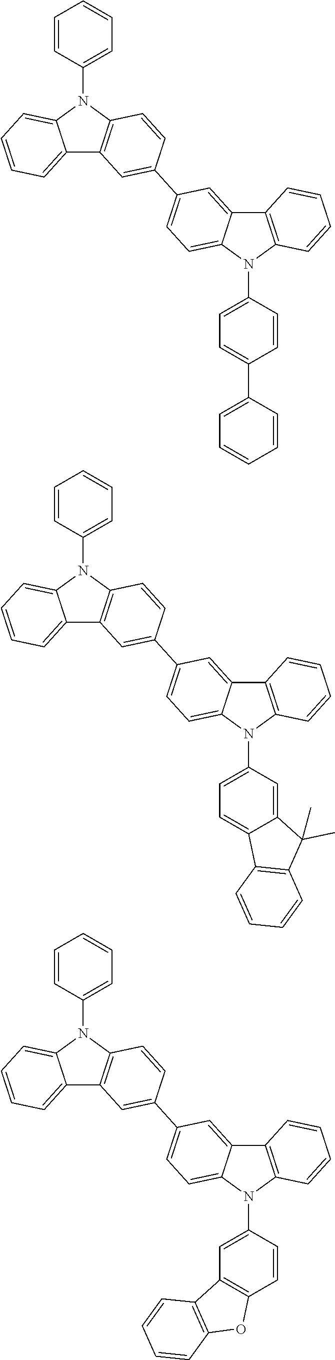 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1]

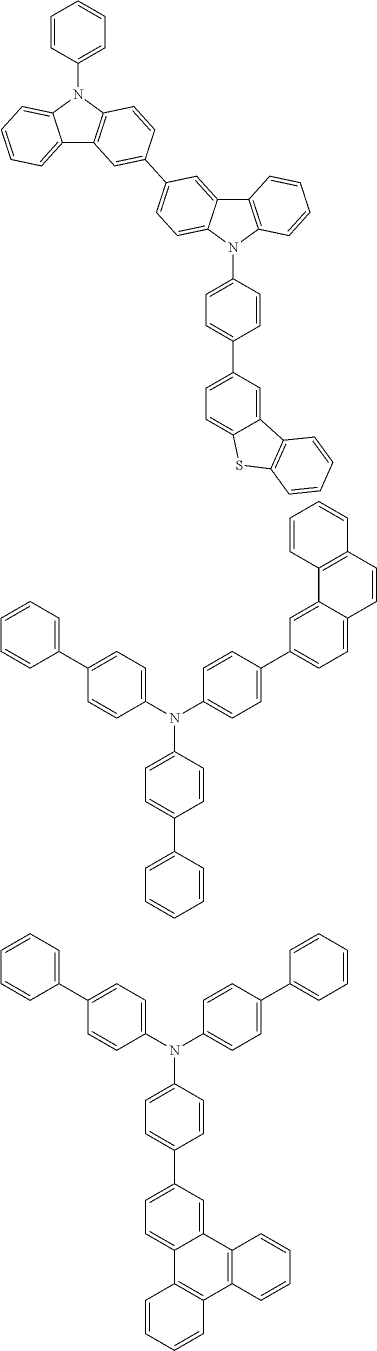

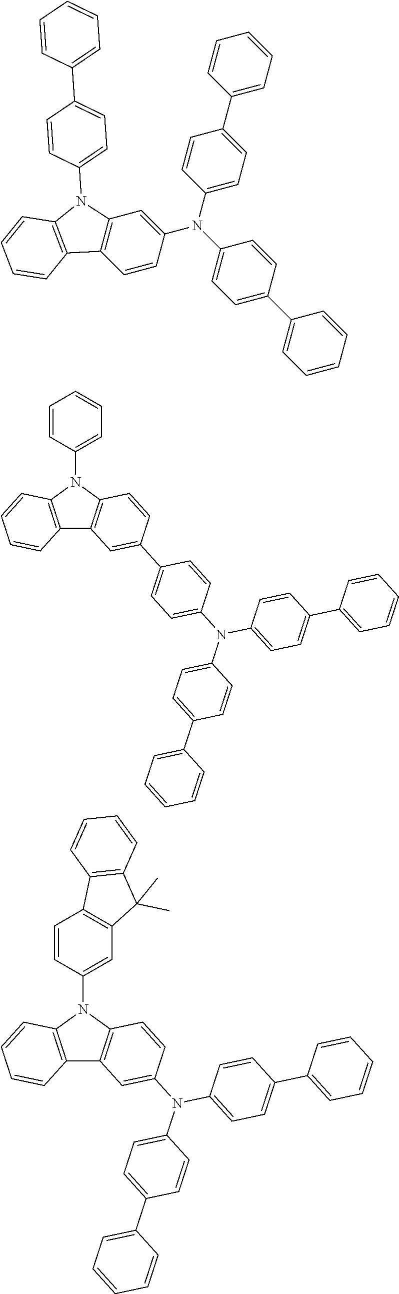

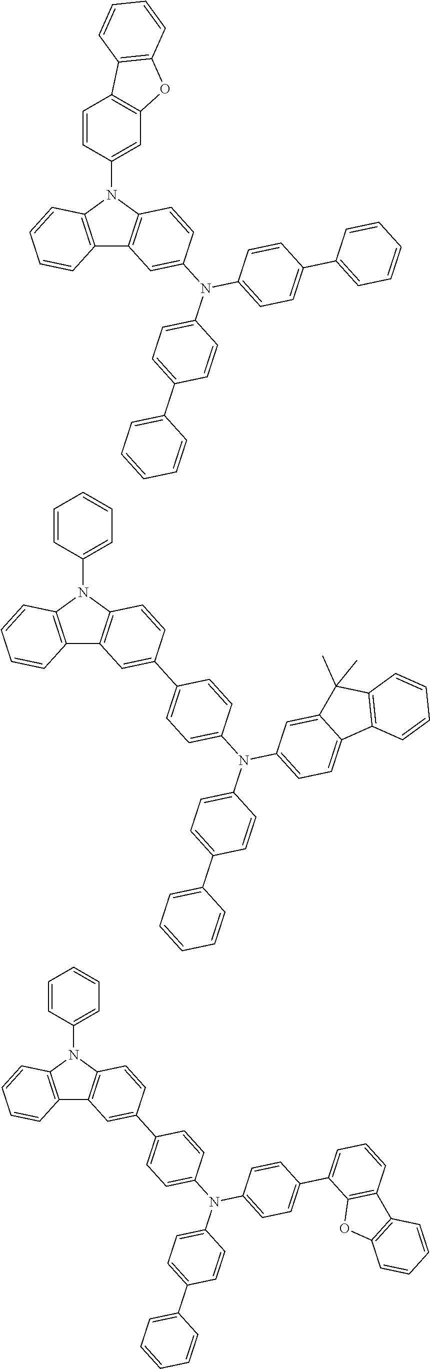

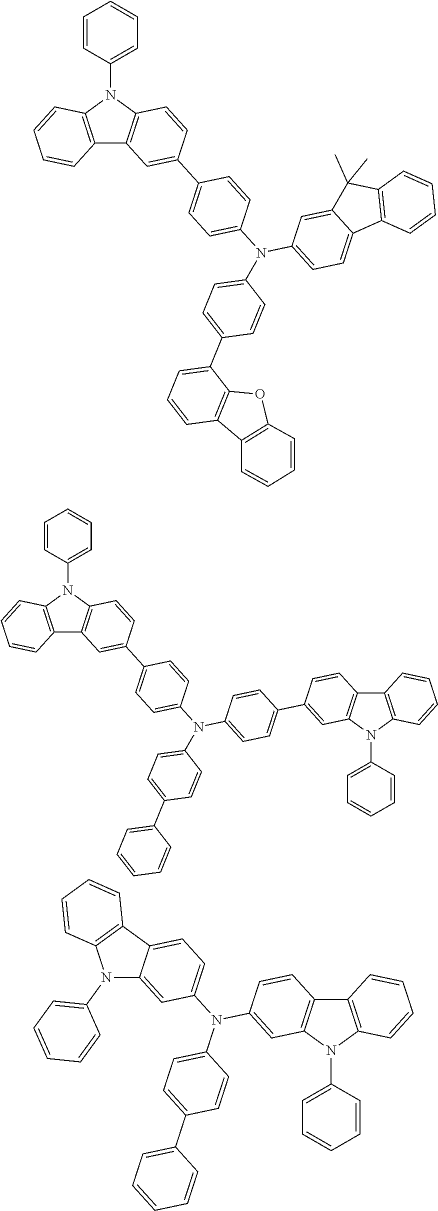

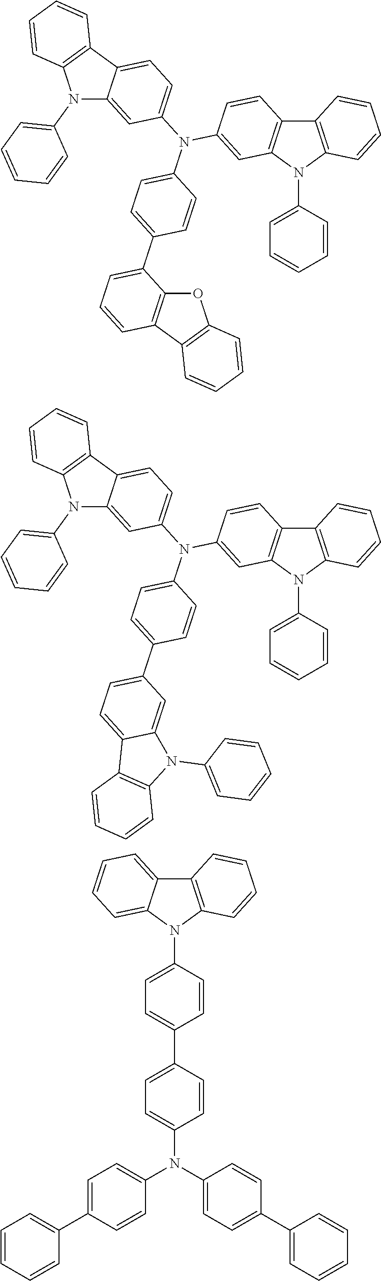

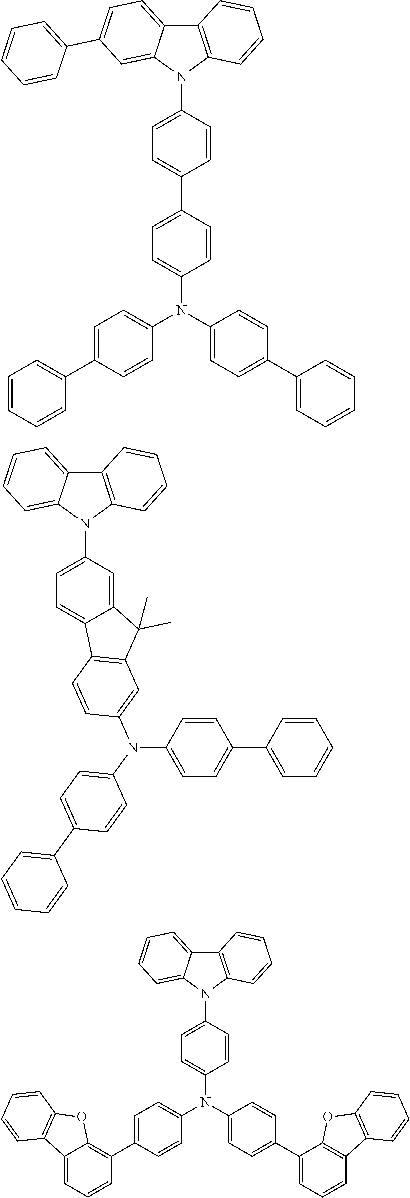

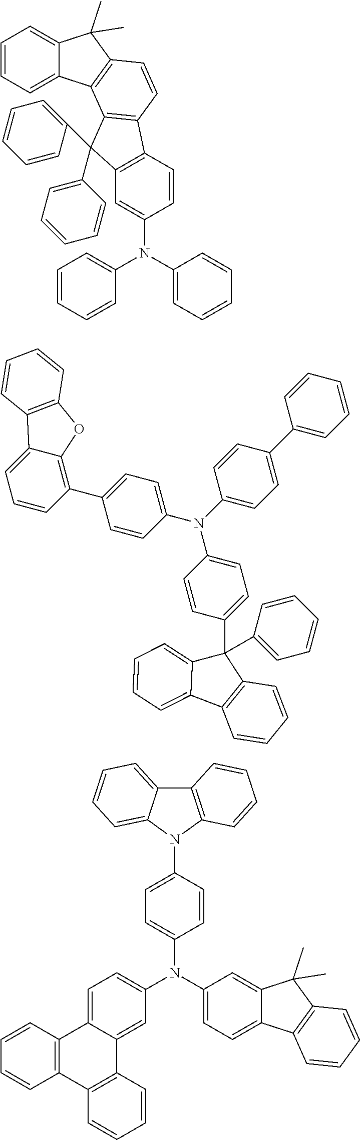

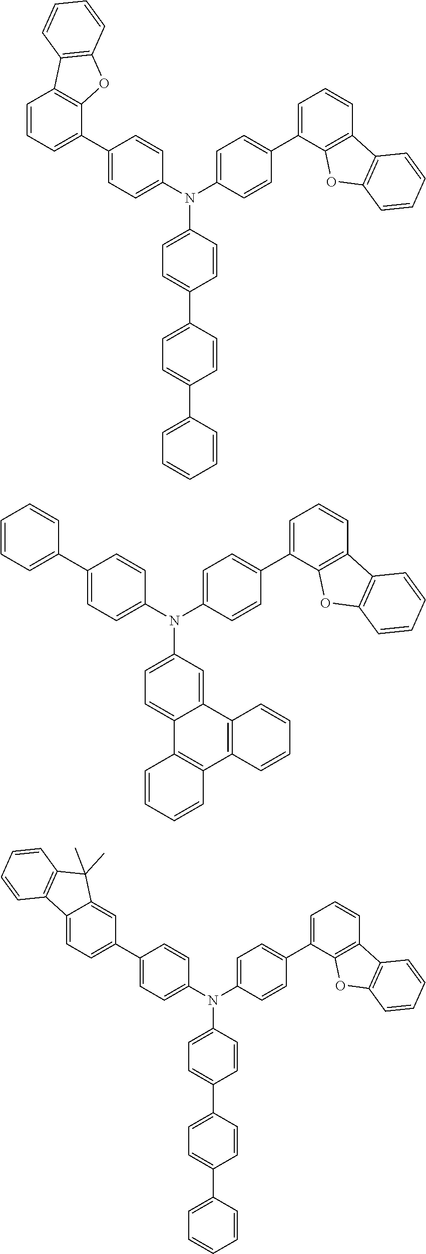 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2ccc(N(c3ccc(-c4cccc5c4oc4ccccc45)cc3)c3ccc4c5ccccc5c5ccccc5c4c3)cc2)cc1 ; CC1(C)c2ccccc2-c2ccc(-c3ccc(N(c4ccc(-c5ccc(-c6ccccc6)cc5)cc4)c4ccc(-c5cccc6c5oc5ccccc56)cc4)cc3)cc21]

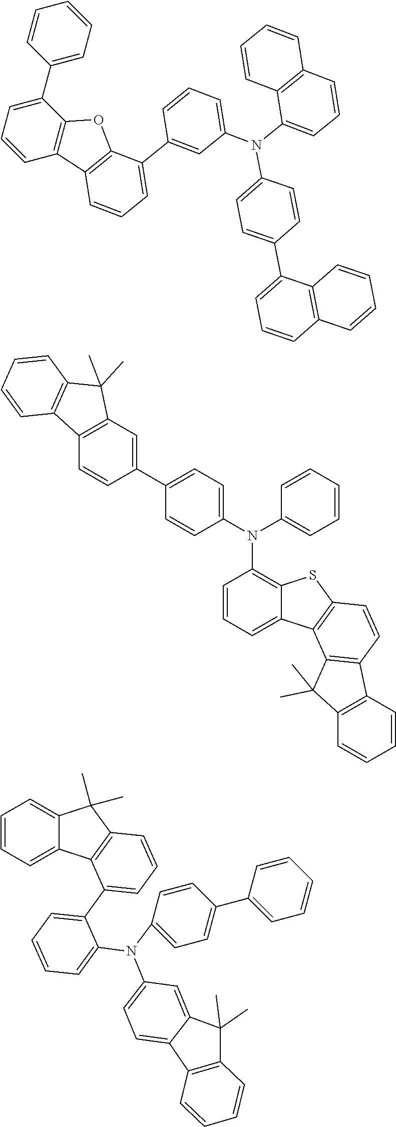 [SMILES: c1ccc(-c2cccc3c2oc2c(-c4cccc(N(c5ccc(-c6cccc7ccccc67)cc5)c5cccc6ccccc56)c4)cccc23)cc1 ; CC1(C)c2ccccc2-c2ccc(-c3ccc(N(c4ccccc4)c4cccc5c4sc4ccc6c(c45)C(C)(C)c4ccccc4-6)cc3)cc21 ; CC1(C)c2ccccc2-c2ccc(N(c3ccc(-c4ccccc4)cc3)c3ccccc3-c3cccc4c3-c3ccccc3C4(C)C)cc21]

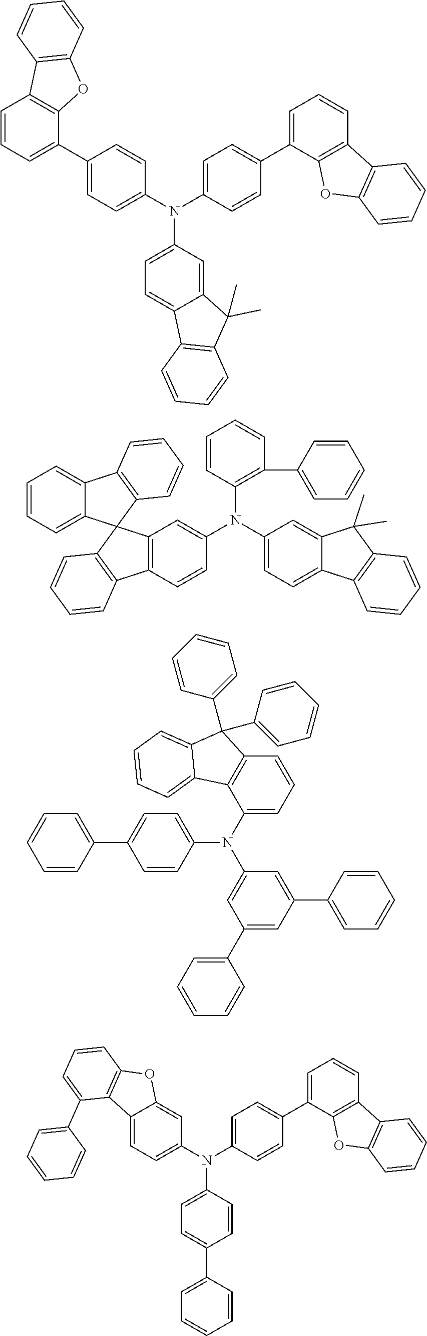

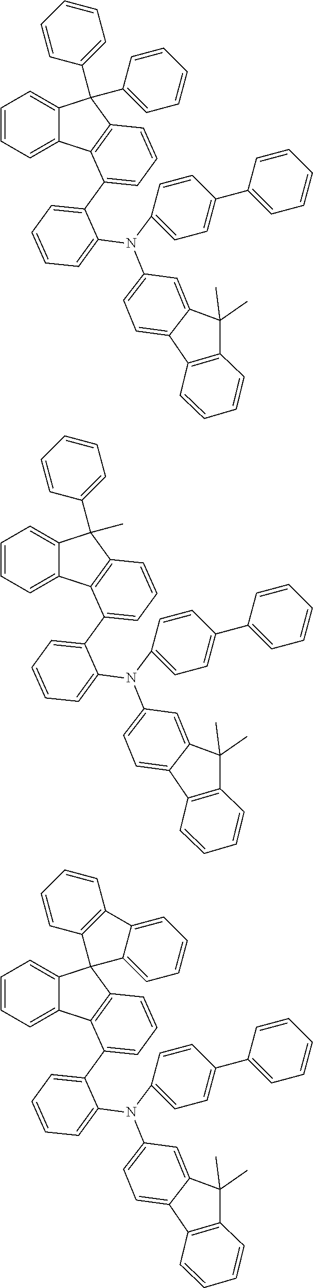 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21 ; CC1(C)c2ccccc2-c2ccc(N(c3ccc(-c4ccccc4)cc3)c3ccccc3-c3cccc4c3-c3ccccc3C4(C)c3ccccc3)cc21 ; CC1(C)c2ccccc2-c2ccc(N(c3ccc(-c4ccccc4)cc3)c3ccccc3-c3cccc4c3-c3ccccc3C43c4ccccc4-c4ccccc43)cc21]

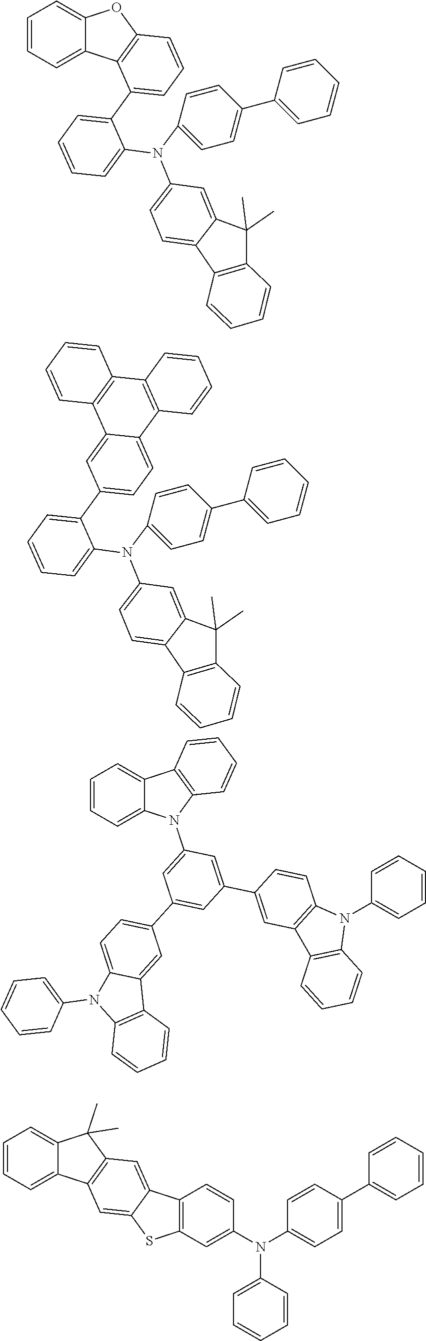

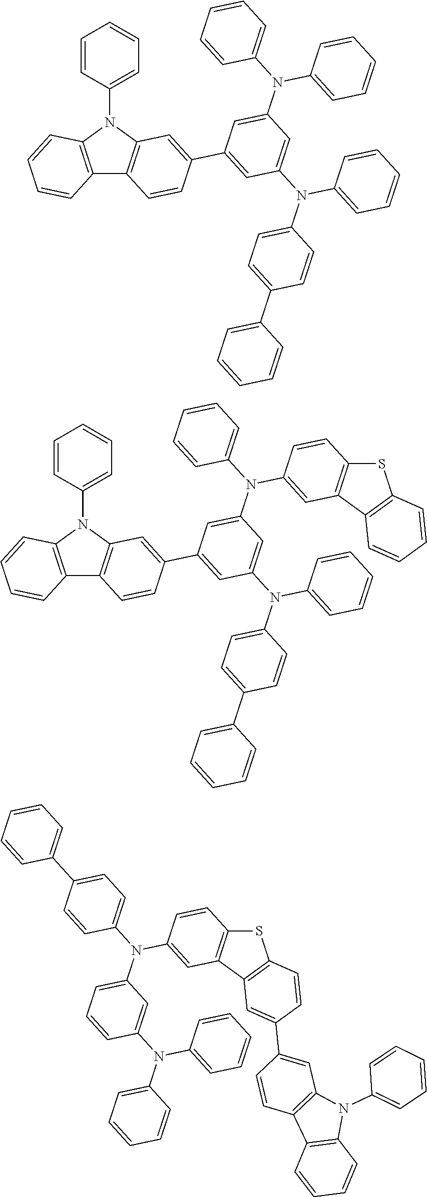

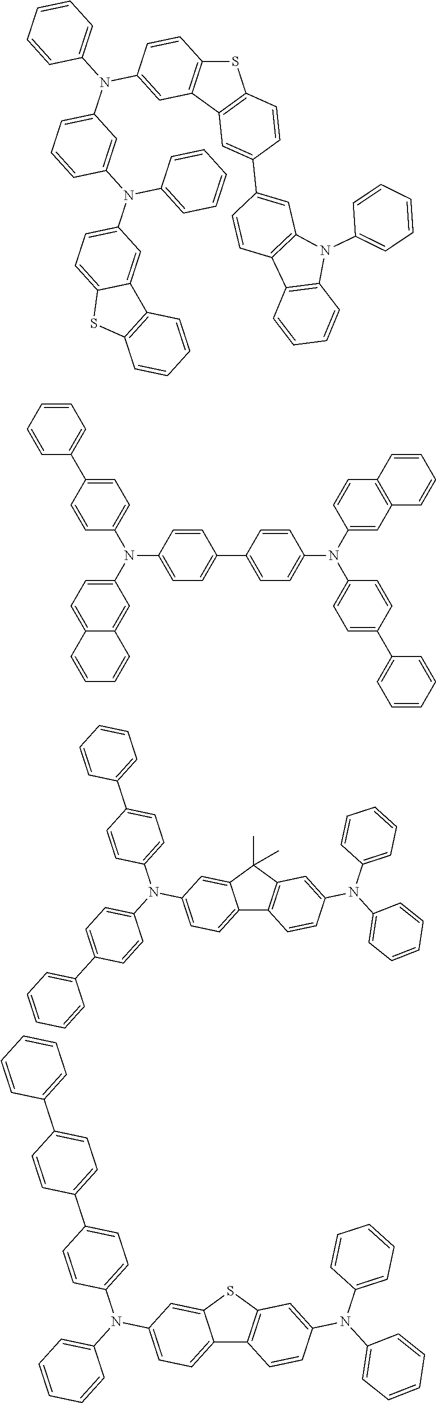

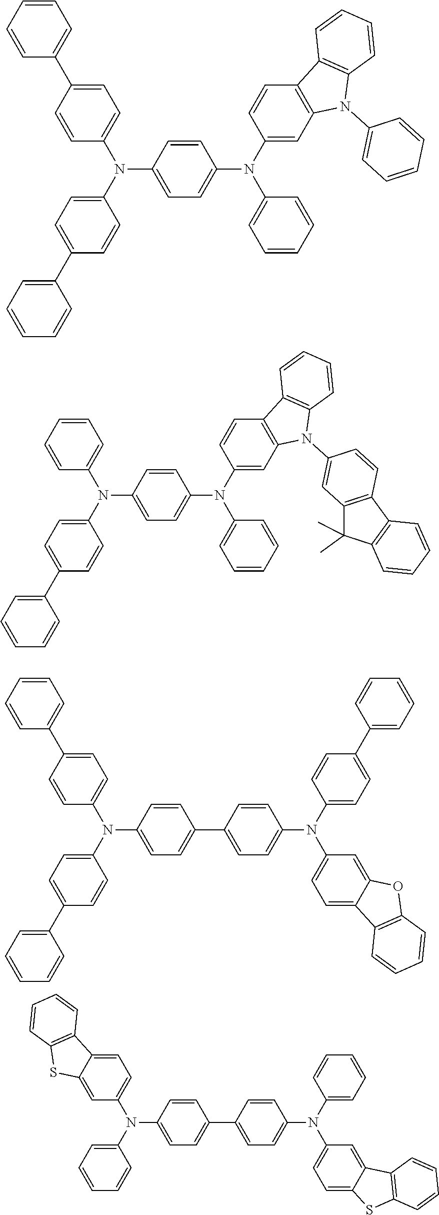

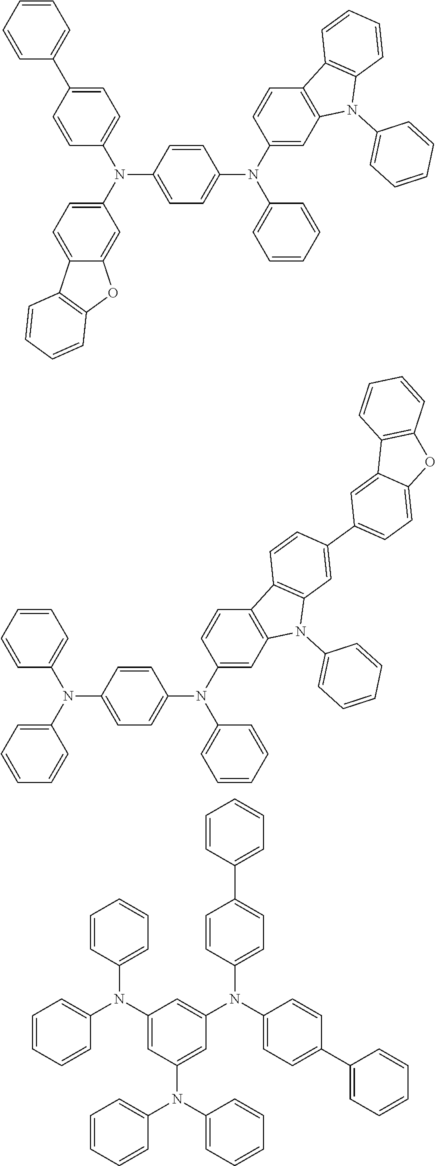

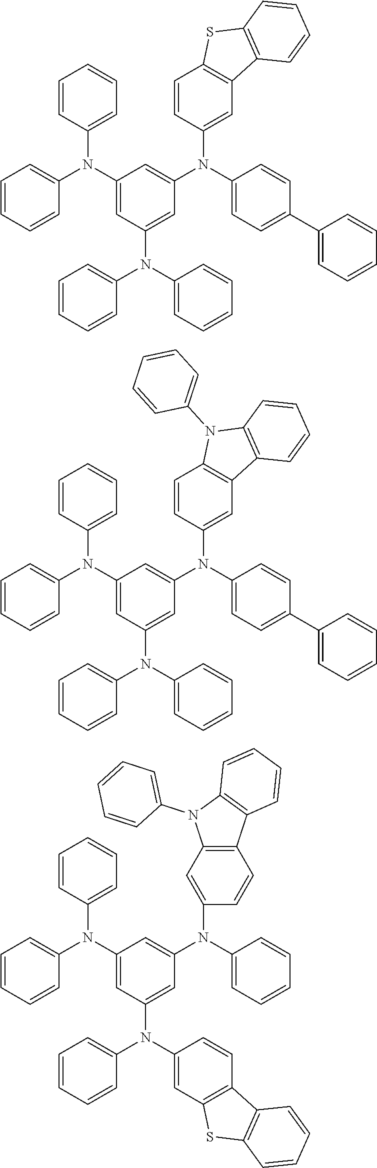

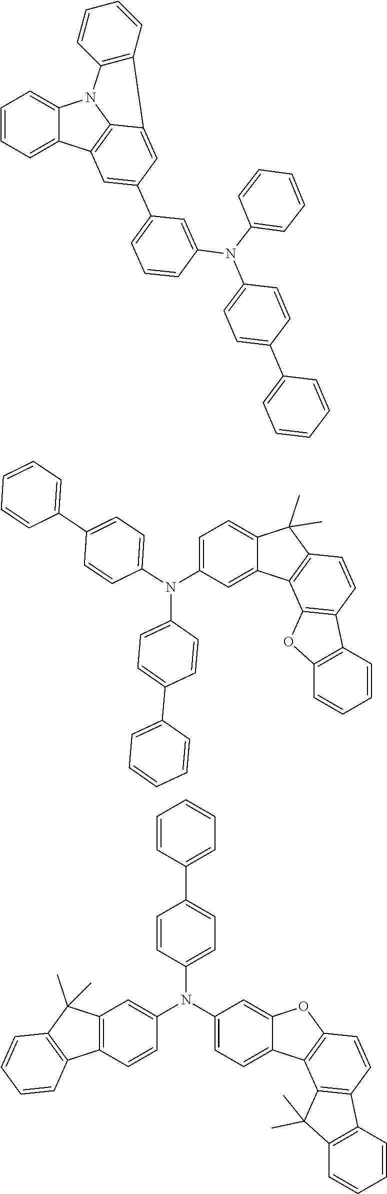

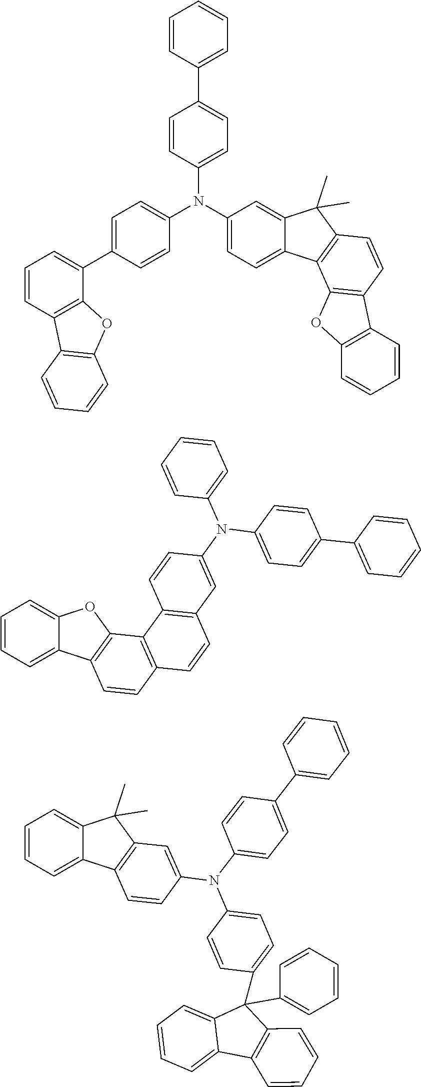 [SMILES: CC1(C)c2cc(N(c3ccc(-c4ccccc4)cc3)c3ccc(-c4cccc5c4oc4ccccc45)cc3)ccc2-c2c1ccc1c2oc2ccccc21 ; c1ccc(-c2ccc(N(c3ccccc3)c3ccc4c(ccc5ccc6c7ccccc7oc6c54)c3)cc2)cc1 ; CC1(C)c2ccccc2-c2ccc(N(c3ccc(-c4ccccc4)cc3)c3ccc(C4(c5ccccc5)c5ccccc5-c5ccccc54)cc3)cc21]

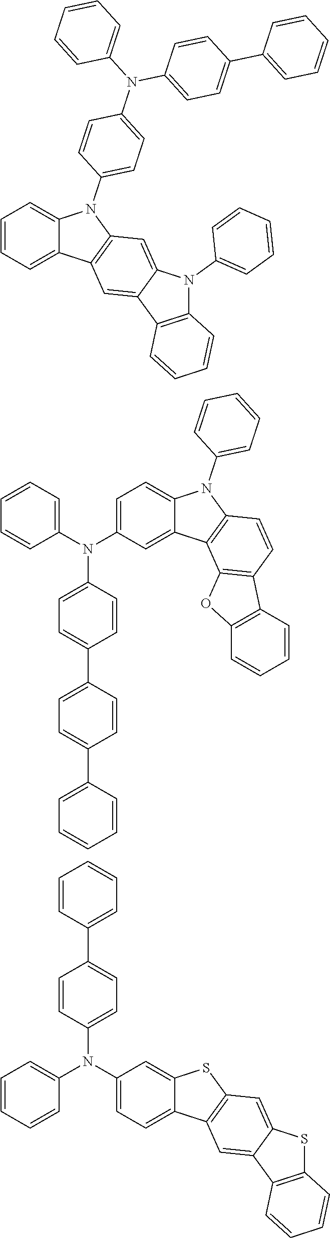

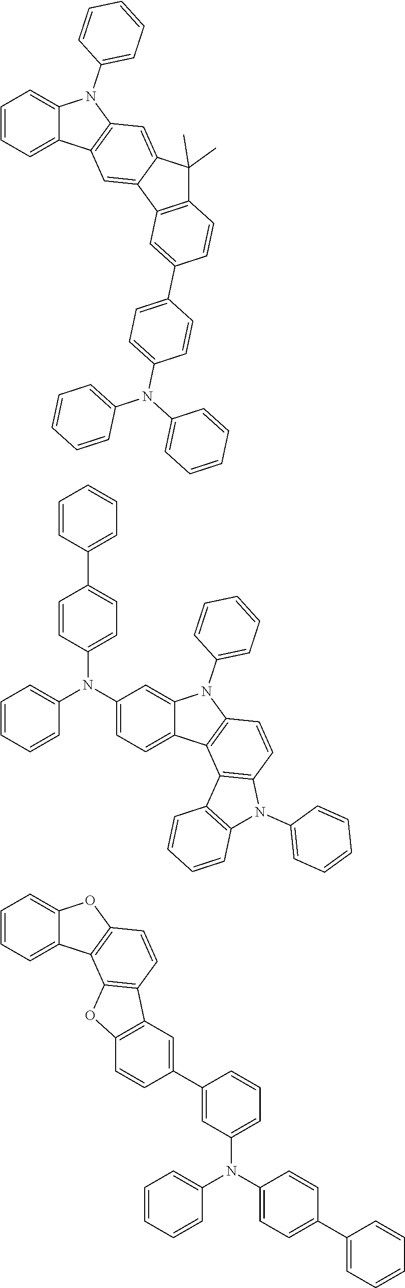

In the hole transport auxiliary layer other suitable compounds may be used in addition to the aforementioned compounds.

In an implementation, in FIG. 1 or 2, an organic light emitting diode may further include an electron transport layer, an electron injection layer, or a hole injection layer as a part of the organic layer 105.

The organic light emitting diodes 100 and 200 may be manufactured by, e.g., forming an anode or a cathode on a substrate, forming an organic layer using a dry film formation method such as a vacuum deposition method (evaporation), sputtering, plasma plating, and ion plating, and forming a cathode or an anode thereon.

The organic light emitting diode may be applied to an organic light emitting display device.

Hereinafter, starting materials and reactants used in Examples and Synthesis Examples were purchased from Sigma-Aldrich Co. Ltd., TCI Inc., Tokyo chemical industry or P&H tech or were synthesized by suitable methods.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

The following synthesis intermediates were synthesized according to suitable methods.

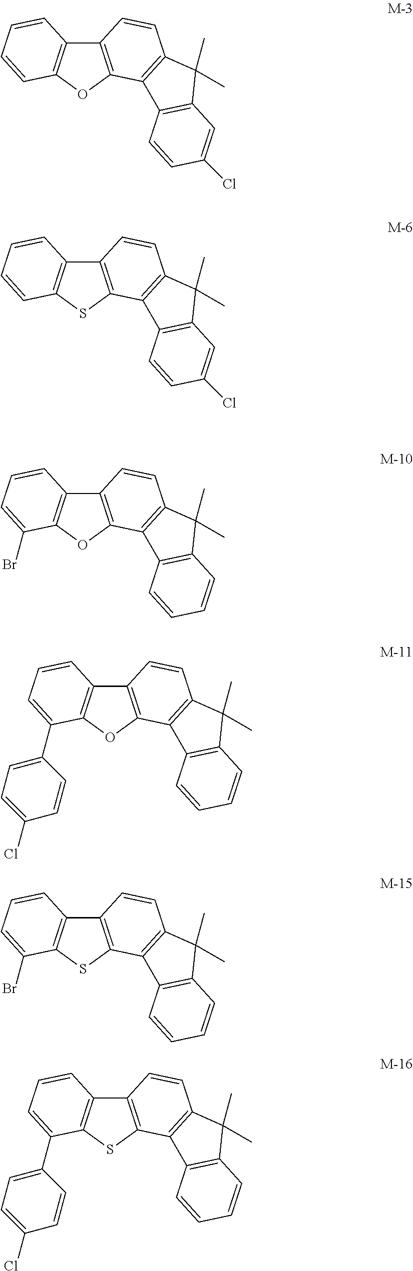

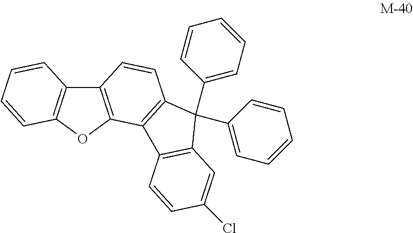 [SMILES: Clc1ccc2c(c1)C(c1ccccc1)(c1ccccc1)c1ccc3c(oc4ccccc43)c1-2]

(Preparation of First Compound)

### Synthesis Example 1: Synthesis of Compound A-51

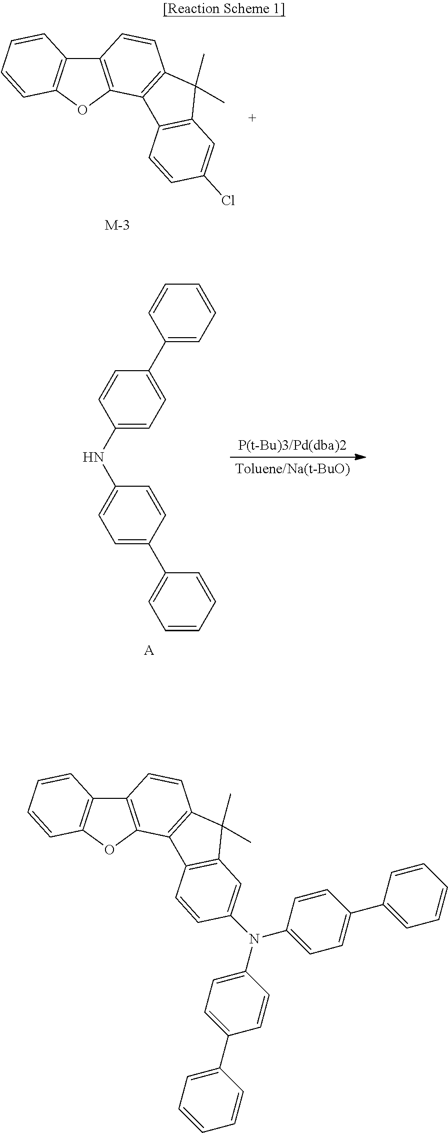

5.0 g (15.68 mmol) of Intermediate M-3, 5.04 g (15.68 mmol) of Intermediate A, 4.52 g (47.95 mmol) of sodium t-butoxide, and 0.1 g (0.47 mmol) of tri-tert-butylphosphine were dissolved in 200 ml of toluene, and 0.27 g (0.47 mmol) of Pd(dba)₂ was added thereto. The mixture was refluxed and stirred under a nitrogen atmosphere for 12 hours. When the reaction was complete, the reactant was extracted with toluene and distilled water. The obtained organic layer was dried with anhydrous magnesium sulfate, filtered, and concentrated under a reduced pressure. The resulting product was purified through silica gel column chromatography using n-hexane/dichloromethane mixed in a volume ratio of 2:1, obtaining a desired compound A-51 as a white solid of 7.8 g (Yield 82.3%).

Calculated value: C, 89.52; H, 5.51; N, 2.32; O, 2.65.

Analyzed value: C, 89.51; H, 5.52; N, 2.32; O, 2.65.

### Synthesis Example 2: Synthesis of Compound A-81

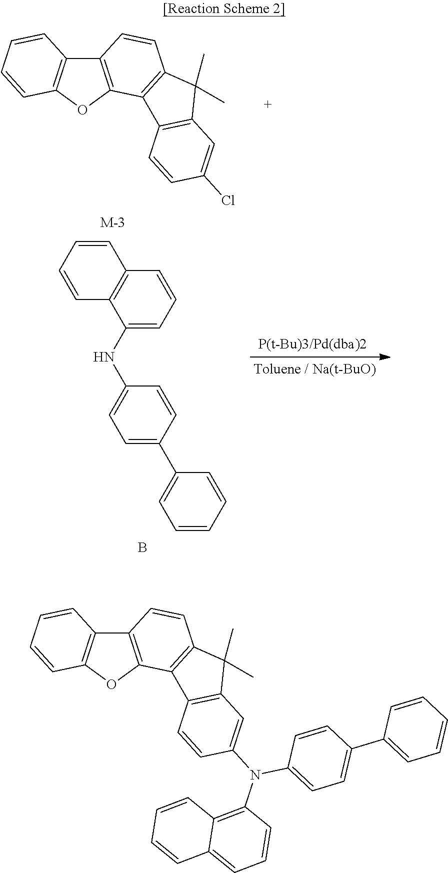

Compound A-81 was synthesized according to the same method as Synthesis Example 1 except that Intermediate M-3 and Intermediate B were used in an equivalence ratio of 1:1 (7.8 g, Yield: 80.5%).

Calculation value: C, 89.40; H, 5.41; N, 2.42; O, 2.77.

Analyzed value: C, 89.42; H, 5.39; N, 2.42; O, 2.77.

### Synthesis Example 3: Synthesis of Compound A-82

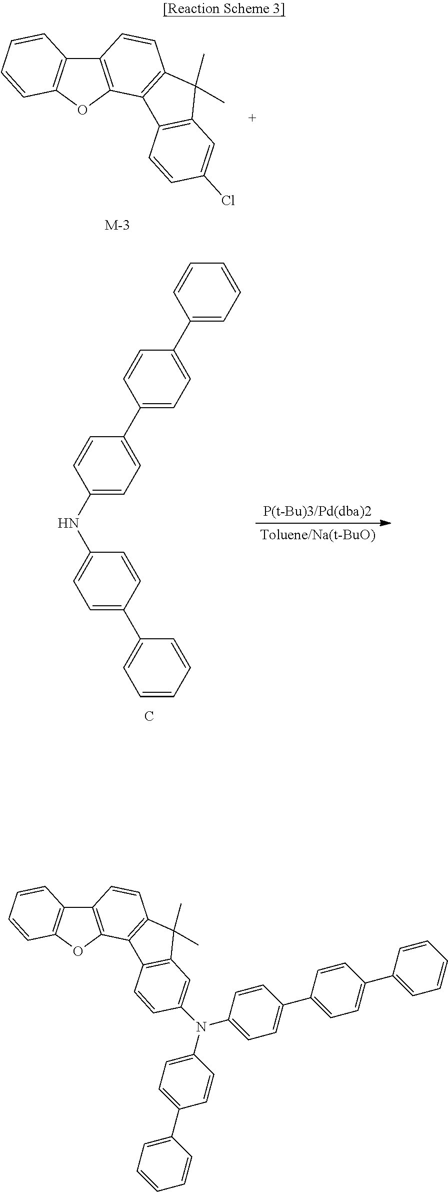

Compound A-82 was synthesized according to the same method as Synthesis Example 1 except that Intermediate M-3 and Intermediate C were used in an equivalence ratio of 1:1 (9.2 g, Yield: 86.2%).

Calculation value: C, 90.10; H, 5.49; N, 2.06; O, 2.35.

Analyzed value: C, 90.12; H, 5.47; N, 2.06; O, 2.35.

### Synthesis Example 4: Synthesis of Compound A-55

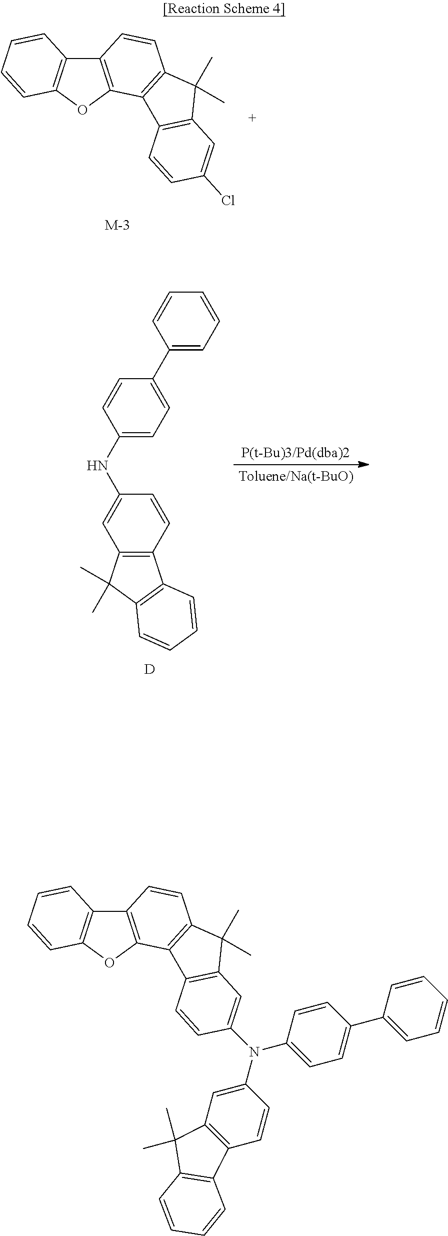

Compound A-55 was synthesized according to the same method as Synthesis Example 1 except that Intermediate M-3 and Intermediate D were used in an equivalence ratio of 1:1 (8.6 g, Yield: 85.1%).

Calculation value: C, 89.55; H, 5.79; N, 2.18; O, 2.49.

Analyzed value: C, 89.56; H, 5.78; N, 2.18; O, 2.49.

### Synthesis Example 5: Synthesis of Compound A-69

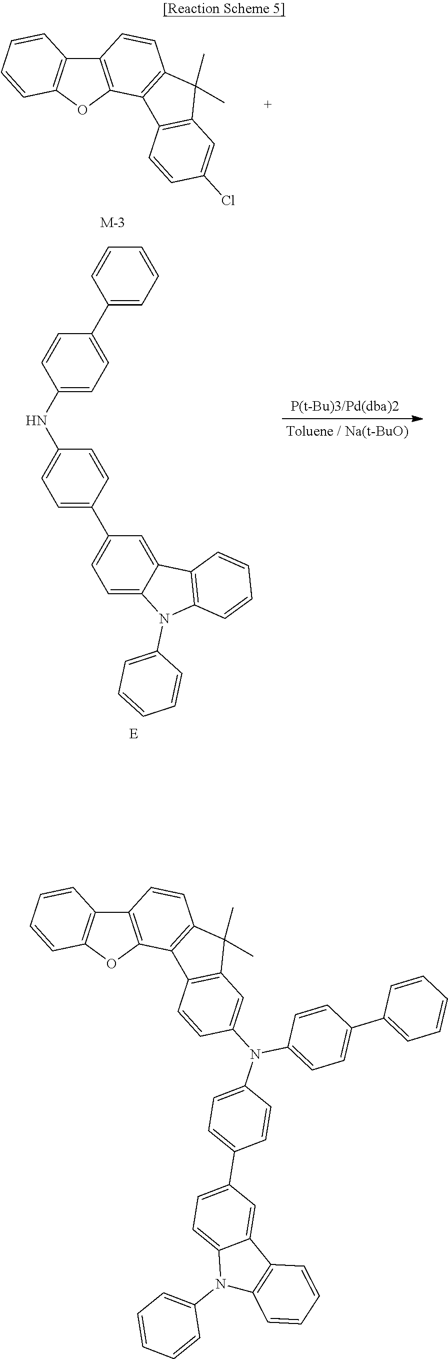

Compound A-69 was synthesized according to the same method as Synthesis Example 1 except that Intermediate M-3 and Intermediate E were used in an equivalence ratio of 1:1 (10.5 g, Yield: 87%).

Calculation value: C, 89.03; H, 5.24; N, 3.64; O, 2.08.

Analyzed value: C, 89.01; H, 5.26; N, 3.64; O, 2.08.

### Synthesis Example 6: Synthesis of Compound A-75

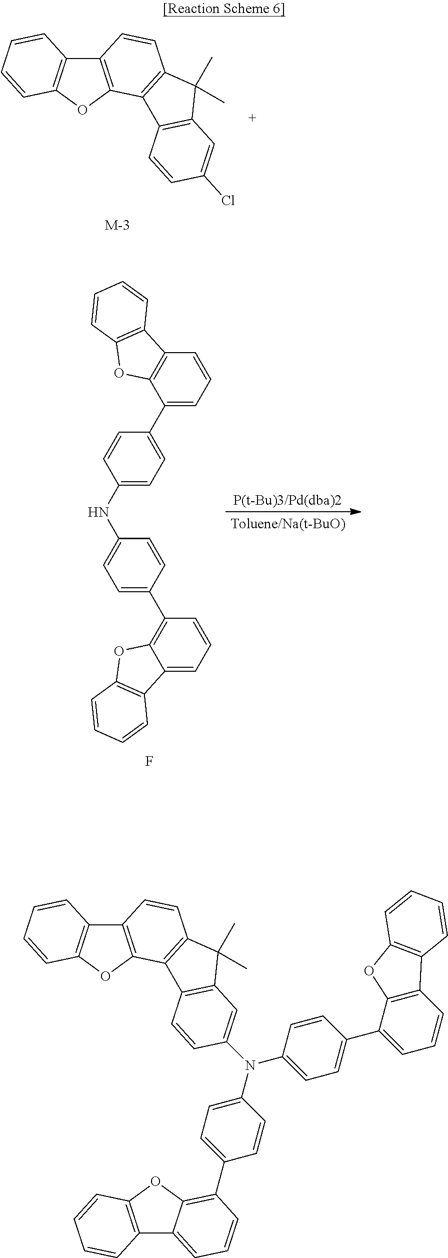

Compound A-75 was synthesized according to the same method as Synthesis Example 1 except that Intermediate M-3 and Intermediate F were used in an equivalence ratio of 1:1 (10.7 g, Yield: 87%).

Calculation value: C, 87.33; H, 4.76; N, 1.79; O, 6.12.

Analyzed value: C, 87.31; H, 4.78; N, 1.79; O, 6.12.

### Synthesis Example 7: Synthesis of Compound A-77

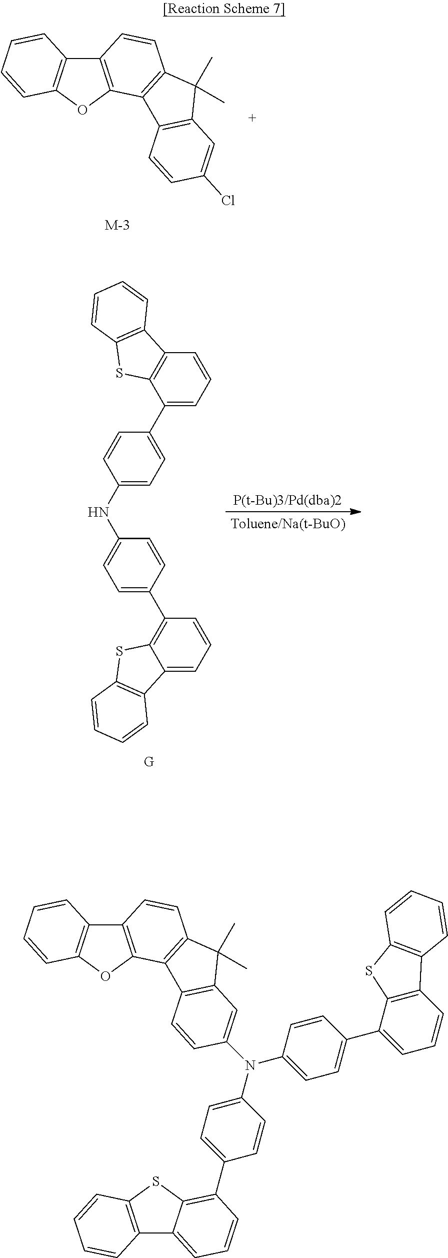

Compound A-77 was synthesized according to the same method as Synthesis Example 1 except that Intermediate M-3 and Intermediate G were used in an equivalence ratio of 1:1 (10.4 g, Yield: 81.2%).

Calculation value: C, 83.89; H, 4.57; N, 1.72; O, 1.96; S, 7.86.

Analyzed value: C, 83.86; H, 4.59; N, 1.72; O, 1.96; S, 7.86.

### Synthesis Example 8: Synthesis of Compound A-79

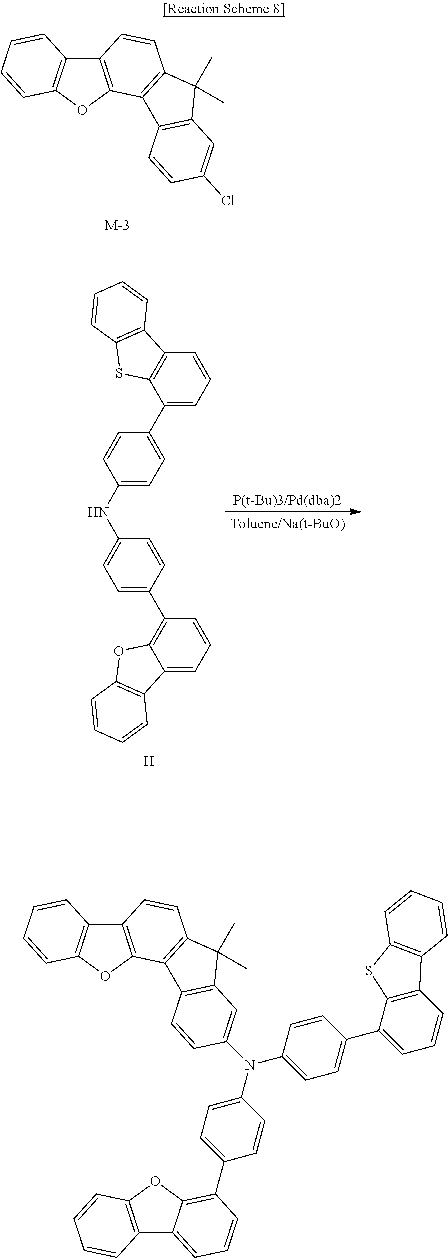

Compound A-79 was synthesized according to the same method as Synthesis Example 1 except that Intermediate M-3 and Intermediate H were used in an equivalence ratio of 1:1 (10.8 g, Yield: 86%).

Calculation value: C, 85.58; H, 4.66; N, 1.75; O, 4.00; S, 4.01.

Analyzed value: C, 85.59; H, 4.67; N, 1.75; O, 4.00; S, 4.01.

### Synthesis Example 9: Synthesis of Compound A-83

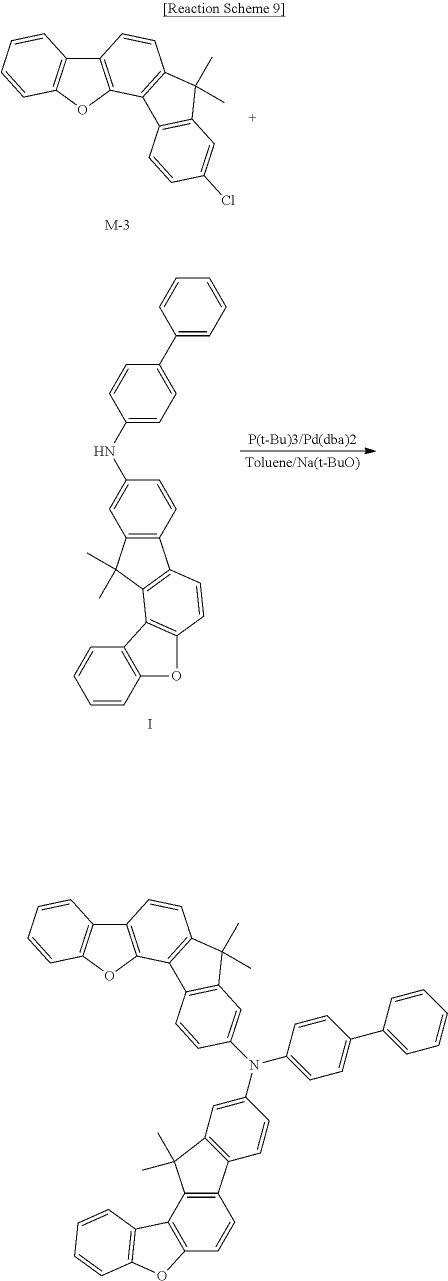

Compound A-83 was synthesized according to the same method as Synthesis Example 1 except that Intermediate M-3 and Intermediate I were used in an equivalence ratio of 1:1 (9.4 g, Yield: 81.6%).

Calculation value: C, 88.37; H, 5.36; N, 1.91; O, 4.36.

Analyzed value: C, 88.35; H, 5.38; N, 1.91; O, 4.36.

### Synthesis Example 10: Synthesis of Compound A-84

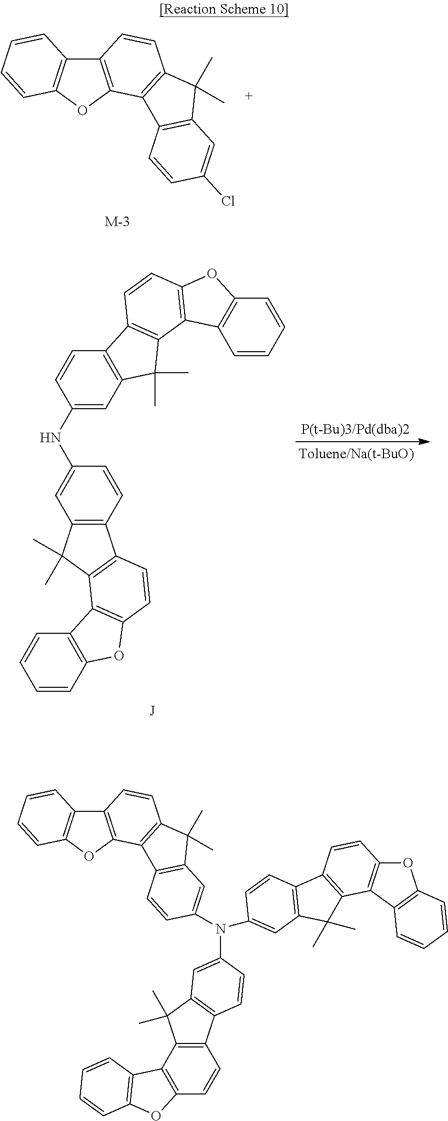 [SMILES: CC1(C)c2cc(Cl)ccc2-c2c1ccc1c2oc2ccccc21 ; "M-3" ; CC1(C)c2cc(Nc3ccc4c(c3)C(C)(C)c3c-4ccc4oc5ccccc5c34)ccc2-c2ccc3oc4ccccc4c3c21 ; CC1(C)c2cc(N(c3ccc4c(c3)C(C)(C)c3c-4ccc4oc5ccccc5c34)c3ccc4c(c3)C(C)(C)c3c-4ccc4oc5ccccc5c34)ccc2-c2c1ccc1c2oc2ccccc21]

Compound A-84 was synthesized according to the same method as Synthesis Example 1 except that Intermediate M-3 and Intermediate J were used in an equivalence ratio of 1:1 (10.4 g, Yield: 76.7%).

Calculation value: C, 87.57; H, 5.25; N, 1.62; O, 5.56.

Analyzed value: C, 87.59; H, 5.23; N, 1.62; O, 5.56.

### Synthesis Example 11: Synthesis of Compound A-52

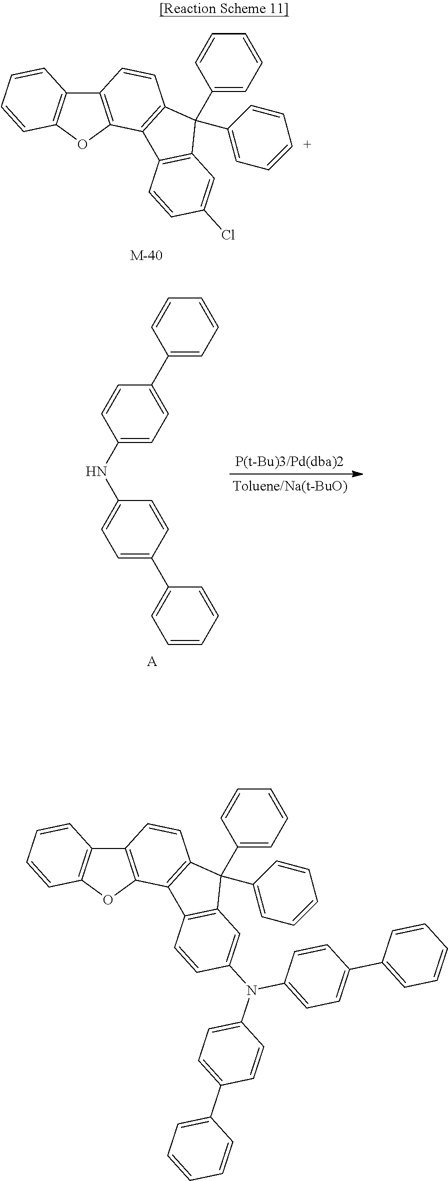

Compound A-52 was synthesized according to the same method as Synthesis Example 1 except that Intermediate M-40 and Intermediate A were used in an equivalence ratio of 1:1 (7.3 g, Yield: 88.8%).

Calculation value: C, 90.75; H, 5.12; N, 1.92; O, 2.20.

Analyzed value: C, 90.73; H, 5.14; N, 1.92; O, 2.20.

### Synthesis Example 12: Synthesis of Compound A-53

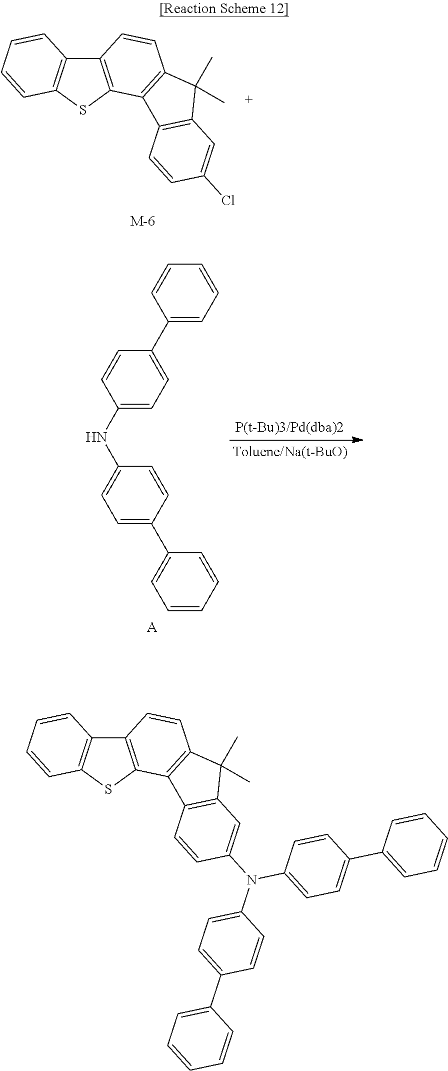

Compound A-53 was synthesized according to the same method as Synthesis Example 1 except that Intermediate M-6 and Intermediate A were used in an equivalence ratio of 1:1 (7.5 g, Yield: 81%).

Calculation value: C, 87.20; H, 5.37; N, 2.26; S, 5.17.

Analyzed value: C, 87.22; H, 5.35; N, 2.26; S, 5.17.

### Synthesis Example 13: Synthesis of Compound A-86

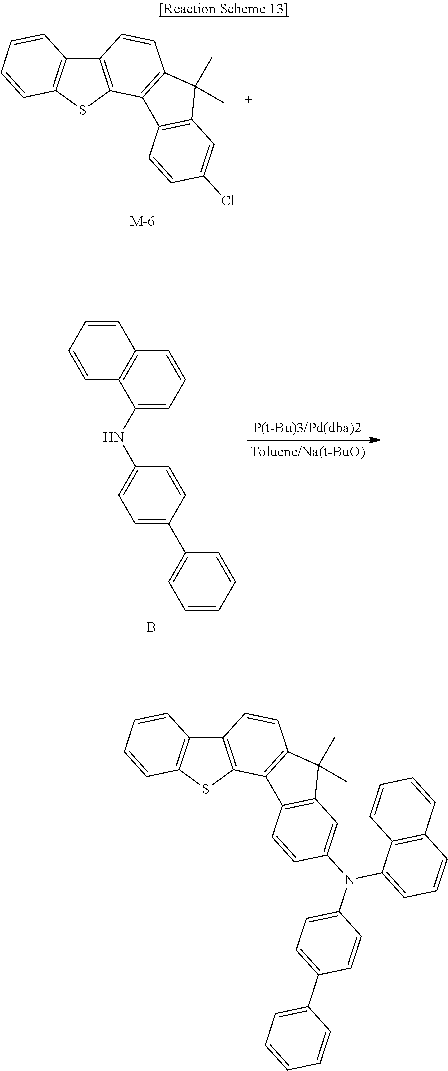

Compound A-86 was synthesized according to the same method as Synthesis Example 1 except that Intermediate M-6 and Intermediate B were used in an equivalence ratio of 1:1 (7.6 g, Yield: 85.7%).

Calculation value: C, 86.98; H, 5.26; N, 2.36; S, 5.40.

Analyzed value: C, 86.99; H, 5.25; N, 2.36; S, 5.40.

### Synthesis Example 14: Synthesis of Compound A-87

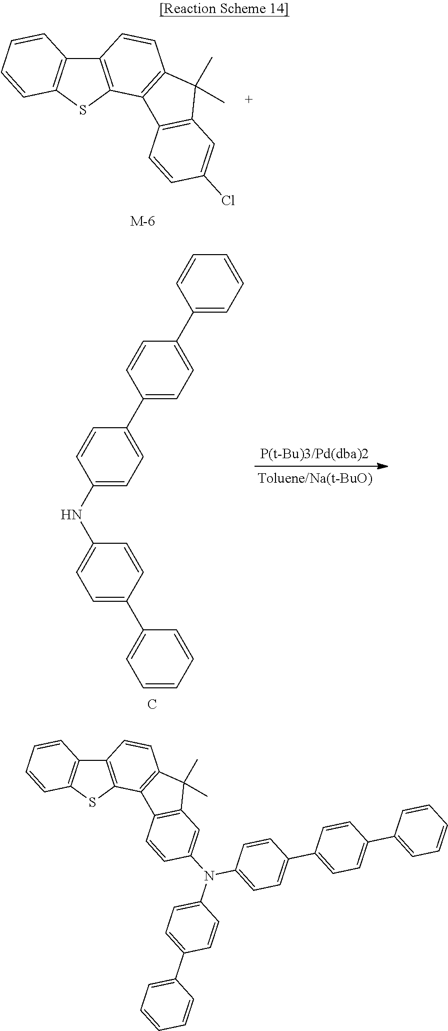

Compound A-87 was synthesized according to the same method as Synthesis Example 1 except that Intermediate M-6 and Intermediate C were used in an equivalence ratio of 1:1 (8.2 g, Yield: 78.9%).

Calculation value: C, 88.02; H, 5.36; N, 2.01; S, 4.61.

Analyzed value: C, 88.00; H, 5.38; N, 2.01; S, 4.61.

### Synthesis Example 15: Synthesis of Compound A-58

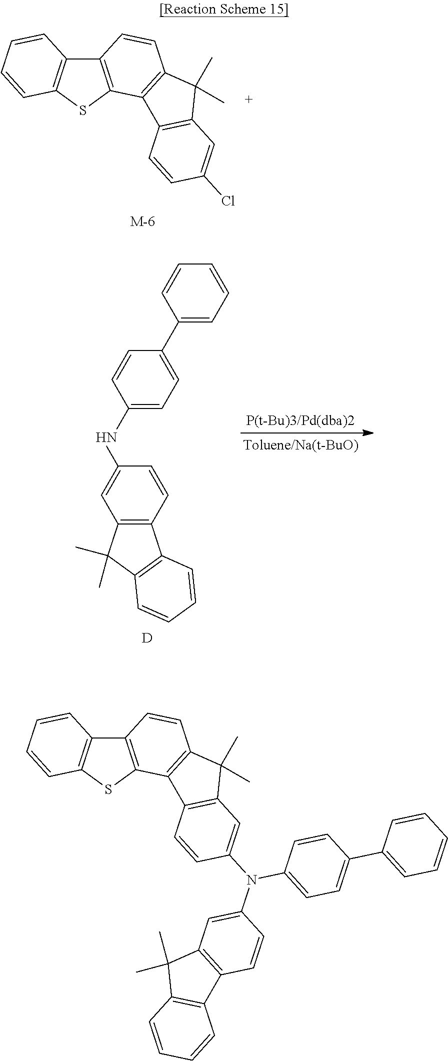

Compound A-58 was synthesized according to the same method as Synthesis Example 1 except that Intermediate M-6 and Intermediate D were used in an equivalence ratio of 1:1 (8.4 g, Yield: 85.2%).

Calculation value: C, 87.37; H, 5.65; N, 2.12; S, 4.86. P Analyzed value: C, 87.35; H, 5.67; N, 2.12; S, 4.86.

### Synthesis Example 16: Synthesis of Compound A-27

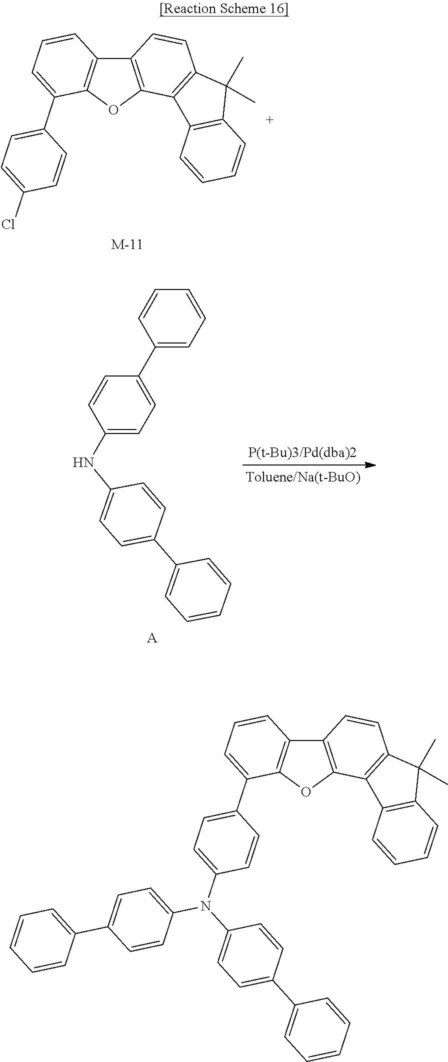

Compound A-27 was synthesized according to the same method as Synthesis Example 1 except that Intermediate M-11 and Intermediate A were used in an equivalence ratio of 1:1 (7.3 g, Yield: 84%).

Calculation value: C, 90.10; H, 5.49; N, 2.06; O, 2.35.

Analyzed value: C, 90.12; H, 5.47; N, 2.06; O, 2.35.

### Synthesis Example 17: Synthesis of Compound A-29

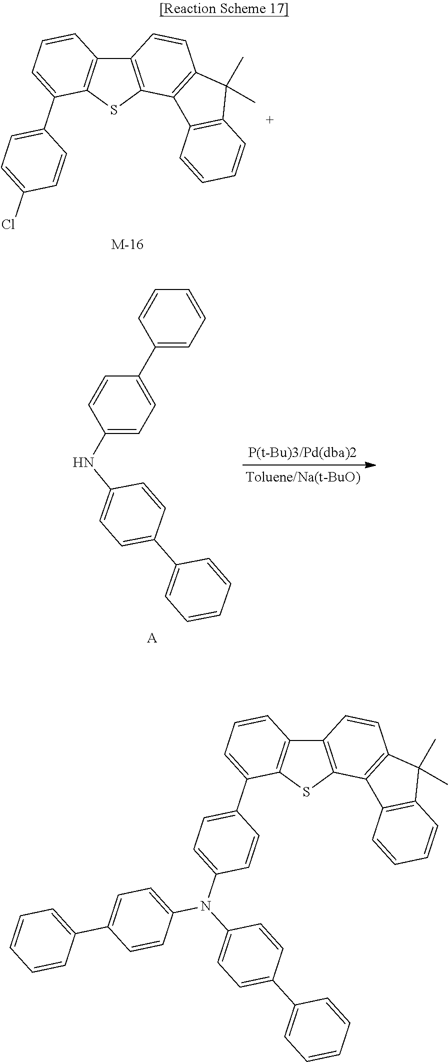

Compound A-29 was synthesized according to the same method as Synthesis Example 1 except that Intermediate M-16 and Intermediate A were used in an equivalence ratio of 1:1 (7.1 g, Yield: 83.8%).

Calculation value: C, 88.02; H, 5.36; N, 2.01; S, 4.61.

Analyzed value: C, 88.04; H, 5.34; N, 2.01; S, 4.61.

### Synthesis Example 18: Synthesis of Compound A-92

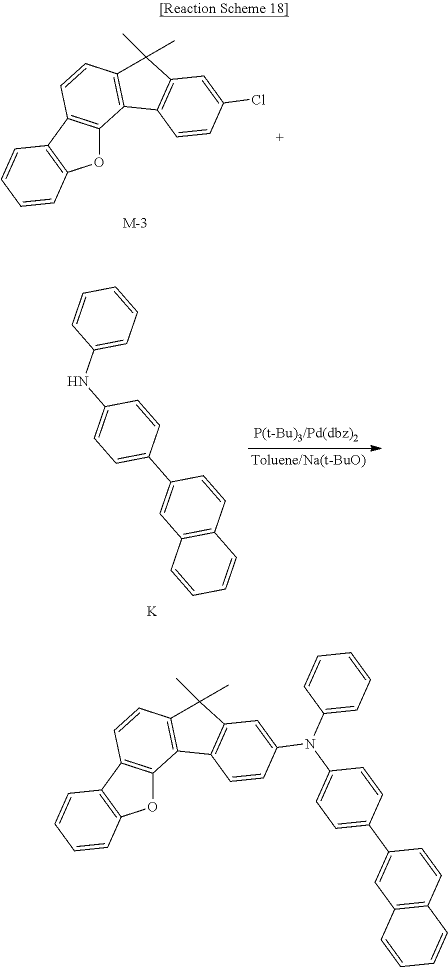

Compound A-92 was synthesized according to the same method as Synthesis Example 1 except that Intermediate M-3 and Intermediate K were used in an equivalence ratio of 1:1.

LC/MS calculated for: C43H31NO Exact Mass: 577.24 found for 577.77 [M+H].

### Synthesis Example 19: Synthesis of Compound A-93

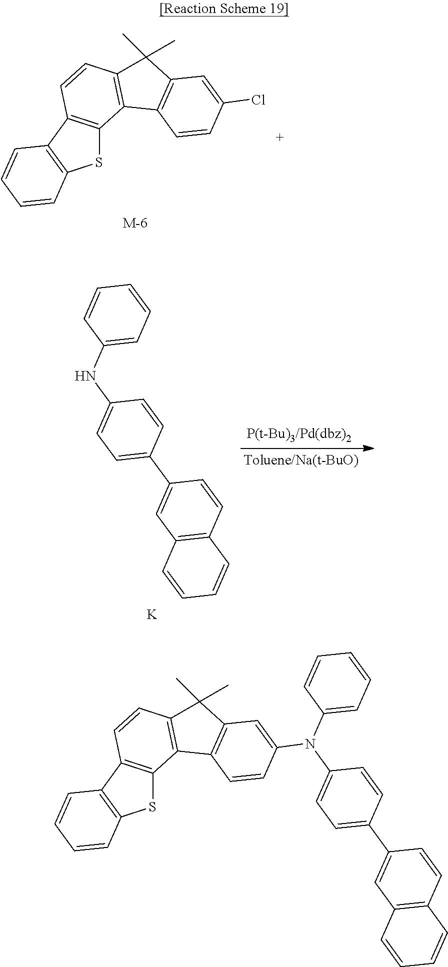

Compound A-93 was synthesized according to the same method as Synthesis Example 1 except that Intermediate M-6 and Intermediate K were used in an equivalence ratio of 1:1.

LC/MS calculated for: C43H31NS Exact Mass: 593.22 found for 593.78 [M+H]

Comparative Synthesis Example 1: Synthesis of Compound V-1

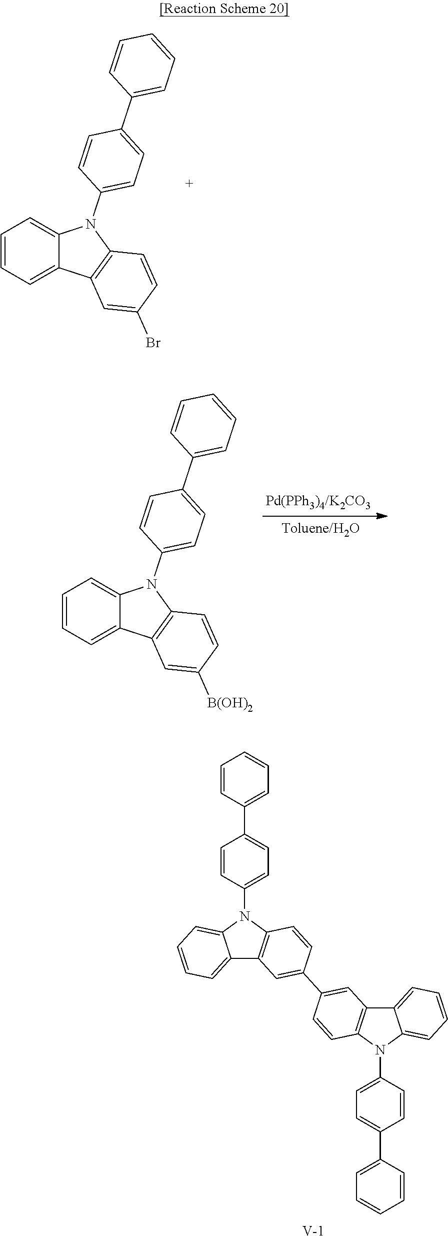

12.33 g (30.95 mmol) of biphenylcarbazolyl bromide was dissolved in 200 mL of toluene under a nitrogen atmosphere, and then 12.37 g (34.05 mmol) of biphenylcarbazolylboronic acid and 1.07 g (0.93 mmol) of tetrakis(triphenylphosphine)palladium were added thereto, and the obtained mixture was stirred. 12.83 g (92.86 mmol) of potassium carbonate saturated in water was added thereto, and the obtained mixture was heated and refluxed at 90° C. for 12 hours. When a reaction was complete, water was added to the reaction solution, and an extract was obtained by using dichloromethane (DCM), filtered after removing moisture therefrom by using anhydrous MgSO₄, and concentrated under a reduced pressure. A residue obtained therefrom was separated and purified through flash column chromatography to obtain Compound V-1 (18.7 g, 92%).

LC/MS calculated for: C48H32N2 Exact Mass: 636.26 found for 636.30 [M+H].

### Comparative Synthesis Example 2: Synthesis of Compound V-2

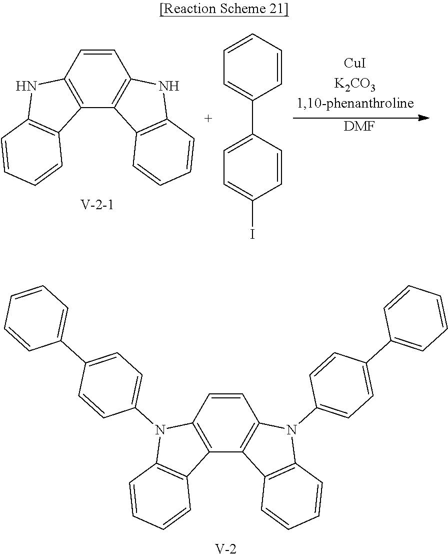

8 g (31.2 mmol) of Intermediate V-2-1 (5,8-dihydro-indolo[2,3-C]carbazole), 20.5 g (73.32 mmol) of 4-iodobiphenyl, 1.19 g (6.24 mmol) of CuI, 1.12 g (6.24 mmol) of 1,10-phenanthoroline, and 12.9 g (93.6 mmol) of K₂CO₃ were put in a round-bottomed flask, 50 ml of DMF was added thereto to dissolve them, and the solution was refluxed and stirred under a nitrogen atmosphere for 24 hours. When a reaction was complete, distilled water was added thereto, and a precipitate therefrom was filtered. The solid was dissolved in 250 ml of xylene, filtered with silica gel, and precipitated as a white solid to obtain 16.2 g of Compound V-2 (Yield: 93%).

LC/MS calculated for: C42H28N2 Exact Mass: 560.23 found for 560.27 [M+H]

(Preparation of Second Compound for Organic Optoelectronic Device)

### Synthesis Example 20 to 26

Compounds B-31, B-32, B-33, B-35, B-36, B-37, and B-56 were synthesized according to a suitable method using the following Starting material 1 and Starting material 2.

TABLE 1

| Syn- | | | | |
|---|---|---|---|---|
| thesis | | | | |
| Exam- | Starting | Starting | | |
| ples | material 1 | material 2 | Product | Yield |
| 20 | 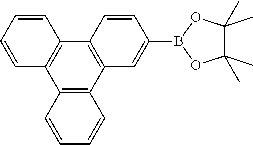 [SMILES: CC1(C)OB(c2ccc3c4ccccc4c4ccccc4c3c2)OC1(C)C] | 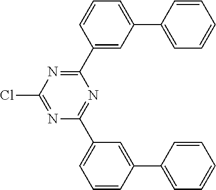 [SMILES: Clc1nc(-c2cccc(-c3ccccc3)c2)nc(-c2cccc(-c3ccccc3)c2)n1] | 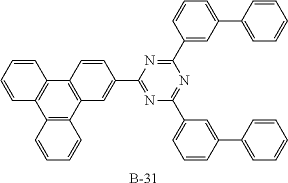 | 78% |
| 21 | 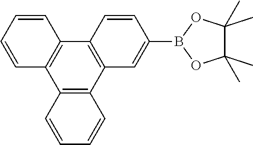 [SMILES: CC1(C)OB(c2ccc3c4ccccc4c4ccccc4c3c2)OC1(C)C] | 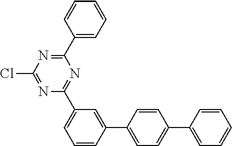 [SMILES: Clc1nc(-c2ccccc2)nc(-c2cccc(-c3ccc(-c4ccccc4)cc3)c2)n1] | 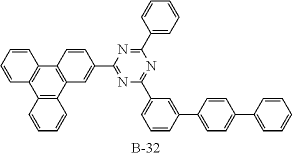 | 80% |
| 22 | 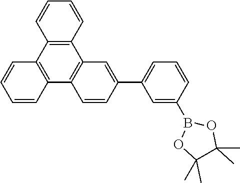 [SMILES: CC1(C)OB(c2cccc(-c3ccc4c5ccccc5c5ccccc5c4c3)c2)OC1(C)C] | 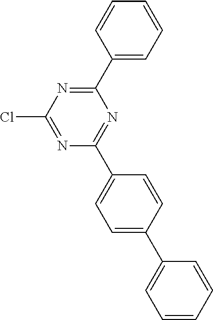 [SMILES: Clc1nc(-c2ccccc2)nc(-c2ccc(-c3ccccc3)cc2)n1] | 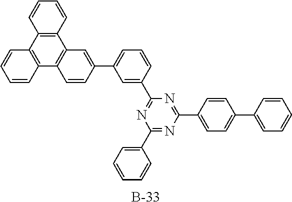 | 79% |
| 23 | 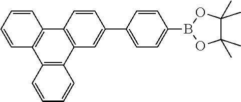 [SMILES: CC1(C)OB(c2ccc(-c3ccc4c5ccccc5c5ccccc5c4c3)cc2)OC1(C)C] | 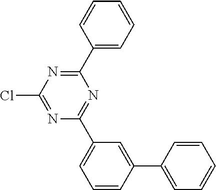 [SMILES: Clc1nc(-c2ccccc2)nc(-c2cccc(-c3ccccc3)c2)n1] | 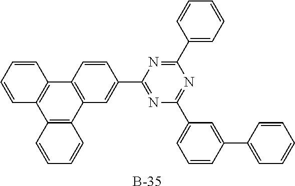 | 81% |
| 24 | 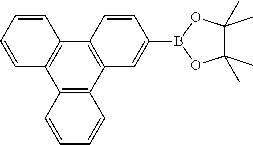 [SMILES: CC1(C)OB(c2ccc3c4ccccc4c4ccccc4c3c2)OC1(C)C] | 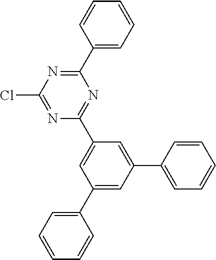 [SMILES: Clc1nc(-c2ccccc2)nc(-c2cc(-c3ccccc3)cc(-c3ccccc3)c2)n1] | 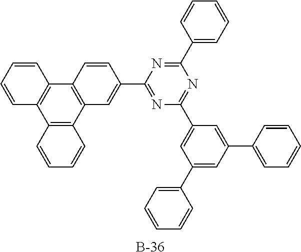 | 83% |
| 25 | 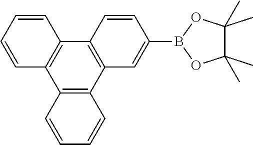 [SMILES: CC1(C)OB(c2ccc3c4ccccc4c4ccccc4c3c2)OC1(C)C] | 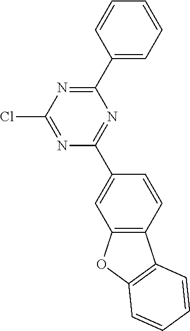 [SMILES: Clc1nc(-c2ccccc2)nc(-c2ccc3c(c2)oc2ccccc23)n1] | 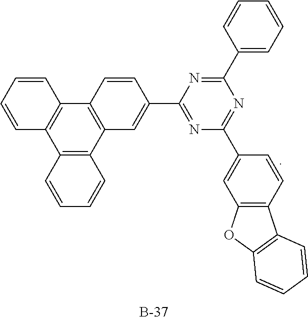 | 85% |
| 26 | 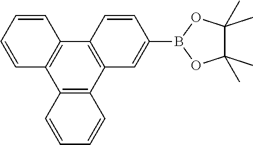 [SMILES: CC1(C)OB(c2ccc3c4ccccc4c4ccccc4c3c2)OC1(C)C] | 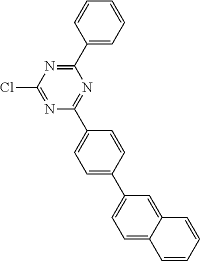 [SMILES: Clc1nc(-c2ccccc2)nc(-c2ccc(-c3ccc4ccccc4c3)cc2)n1] | 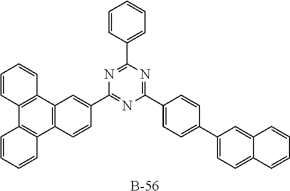 | 88% |

### Synthesis Example 27: Synthesis of Compound B-6

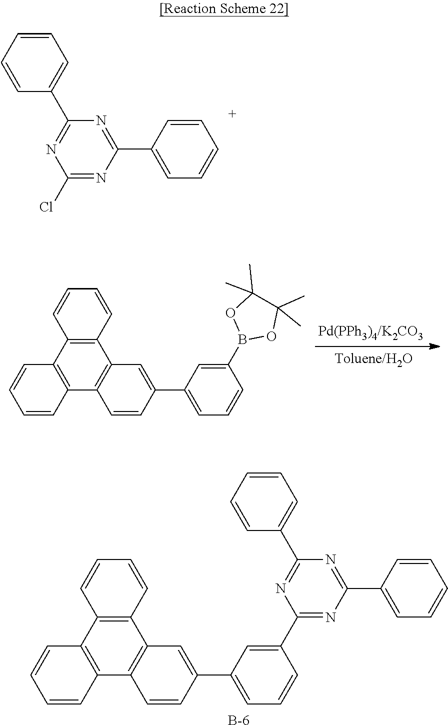

Compound B-6 was synthesized according to the same method as Synthesis Examples 20 to 26 using 2-chloro-4,6-diphenyl-1,3,5-triazine and 4,4,5,5-tetramethyl-2-[3-(2-triphenylenyl)phenyl]-1,3,2-dioxaborolane in an equivalence ratio of 1:1.1 (Yield: 85%)

LC/MS calculated for: C46H32N2 Exact Mass: 535.20 found for 536.18 [M+H].

(Manufacture of Organic Light Emitting Diode)

### Example 1

A glass substrate coated with ITO (indium tin oxide) as a 1,500 Å-thick thin film was washed with distilled water. After washing with the distilled water, the glass substrate was ultrasonic wave-washed with isopropyl alcohol, acetone, or methanol, and dried and then, moved to a plasma cleaner, cleaned by using oxygen plasma for 10 minutes, and moved to a vacuum depositor. This obtained ITO transparent electrode was used as an anode, Compound A was vacuum-deposited on the ITO substrate to form a 700 Å-thick hole injection layer, and Compound B was deposited to be 50 Å-thick on the injection layer, and then Compound C was deposited to be 700 Å-thick to form a hole transport layer. On the hole transport layer, a 400 Å-thick hole transport auxiliary layer was formed by vacuum-depositing Compound C-1. On the hole transport auxiliary layer, 400 Å-thick light emitting layer was formed by using Compounds A-51 and B-35 simultaneously as a host and doping 2 wt % of [Ir(piq)₂acac] as a dopant by a vacuum-deposition. Herein Compound A-51 and Compound B-35 were used in a weight ratio of 7:3 and their ratios in the following Examples were separately provided. Subsequently, on the light emitting layer, a 300 Å-thick electron transport layer was formed by simultaneously vacuum-depositing Compound D and Liq in a ratio of 1:1, and on the electron transport layer, Liq and Al were sequentially vacuum-deposited to be 15 Å-thick and 1,200 Å-thick, manufacturing an organic light emitting diode.

The organic light emitting diode had a five-layered organic thin layer, and specifically the following structure.

ITO/Compound A (700 Å)/Compound B (50 Å)/Compound C (700 Å)/Compound C-1 (400 Å)/EML [Compound A-51: B-35: [Ir(piq)₂acac] (2 wt %)] (400 Å)/Compound D: Liq (300 Å)/Liq (15 Å)/Al (1200 Å).

Compound A: N4,N4′-diphenyl-N4,N4′-bis(9-phenyl-9H-carbazol-3-yl)biphenyl-4,4′-diamine

Compound B: 1,4,5,8,9,11-hexaazatriphenylene-hexacarbonitrile (HAT-CN)

Compound C: N-(biphenyl-4-yl)-9,9-dimethyl-N-(4-(9-phenyl-9H-carbazol-3-yl)phenyl)-9H-fluoren-2-amine

Compound C-1: N,N-di([1,1′-biphenyl]-4-yl)-7,7-dimethyl-7H-fluoreno[4,3-b]benzofuran-10-amine

Compound D: 8-(4-(4,6-di(naphthalen-2-yl)-1,3,5-triazin-2-yl)phenyl)quinoline

### Example 2 to Example 15, Comparative Example 1, and Comparative Example 2

Each organic light emitting diode was manufactured according to the same method as Example 1 except for changing compositions as shown in Table 2.

Evaluation

Power efficiency of the organic light emitting diodes according to Examples 1 to 15 and Comparative Examples 1 and 2 were evaluated.

Specific measurement methods are as follows, and the results are shown in Table 2.

(1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

(2) Measurement of Luminance Change Depending on Voltage Change

Luminance was measured by using a luminance meter (Minolta Cs-1000A), while the voltage of the organic light emitting diodes was increased from 0 V to 10 V.

(3) Measurement of Power Efficiency

Power efficiency (cd/A) at the same current density (10 mA/cm²) were calculated by using the luminance, current density, and voltages (V) from the items (1) and (2).

(4) Measurement of Life-Span

The results were obtained by measuring a time when current efficiency (cd/A) was decreased down to 97%, while luminance (cd/m²) was maintained to be 9,000 cd/m².

(5) Measurement of Driving Voltage

A driving voltage of each diode was measured using a current-voltage meter (Keithley 2400) at 15 mA/cm².

TABLE 2

| | | | First host: | | Power | Driving | Life-span |
|---|---|---|---|---|---|---|---|
| | First | Second | Second host | | efficiency | voltage | T97 |
| | host | host | Ratio (wt:wt) | Color | (cd/A) | (V) | (h) |
| Example 1 | A-51 | B-35 | 7:3 | red | 21.9 | 3.95 | 105 |
| Example 2 | A-53 | B-35 | 6:4 | red | 22.0 | 3.93 | 95 |
| Example 3 | A-55 | B-35 | 6:4 | red | 22.1 | 3.90 | 95 |
| Example 4 | A-58 | B-35 | 7:3 | red | 22.0 | 3.91 | 110 |
| Example 5 | A-81 | B-35 | 7:3 | red | 23.1 | 3.95 | 70 |
| Example 6 | A-92 | B-35 | 7:3 | red | 23.0 | 3.89 | 140 |
| Example 7 | A-92 | B-35 | 6:4 | red | 23.2 | 3.85 | 130 |
| Example 8 | A-92 | B-56 | 7:3 | red | 22.8 | 3.90 | 140 |
| Example 9 | A-92 | B-56 | 6:4 | red | 22.9 | 3.87 | 125 |
| Example 10 | A-93 | B-31 | 7:3 | red | 22.0 | 3.94 | 100 |
| Example 11 | A-93 | B-33 | 7:3 | red | 22.2 | 4.00 | 80 |
| Example 12 | A-93 | B-35 | 7:3 | red | 22.6 | 3.86 | 150 |
| Example 13 | A-93 | B-35 | 6:4 | red | 22.9 | 3.80 | 140 |
| Example 14 | A-93 | B-56 | 7:3 | red | 22.4 | 3.85 | 145 |
| Example 15 | A-93 | B-56 | 6:4 | red | 22.6 | 3.79 | 125 |
| Comparative | V-1 | B-35 | 5:5 | red | 14.9 | 4.84 | 5 |
| Example 1 | | | | | | | |
| Comparative | V-2 | B-35 | 5:5 | red | 17.7 | 4.12 | 35 |
| Example 2 | | | | | | | |

Referring to Table 2, organic light emitting diodes according to Examples 1 to 15 exhibited greatly improved driving voltage, efficiency, and life-span, compared with the organic light emitting diodes according to Comparative Examples 1 and 2.

One or more embodiments may provide a composition for an organic optoelectronic device capable of realizing a high-efficiency and long life-span organic optoelectronic device.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A composition for an organic optoelectronic device, the composition comprising:
a first compound represented by a combination of Chemical Formula 1 and Chemical Formula 2, and
a second compound represented by Chemical Formula 3: 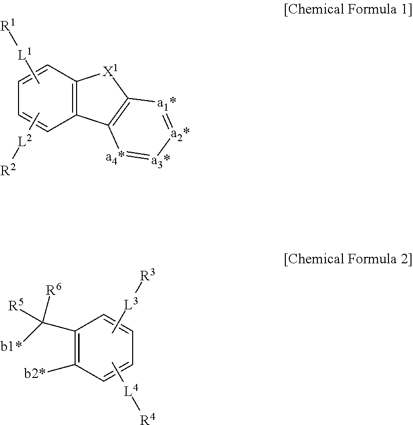
wherein, in Chemical Formula 1 and Chemical Formula 2,
X¹ is O or S,
adjacent two of a₁* to a₄* are C linked with b₁* and b₂* respectively,
the other two of a₁* to a₄* not linked with b₁* and b₂* are each independently C-L^{a}-R^{a},
L^{a} and L¹ to L⁴ are each independently a single bond, a substituted or unsubstituted C6 to C20 arylene group, a substituted or unsubstituted C2 to C20 heterocyclic group, or a combination thereof,
R^{a} and R¹ to R⁶ are each independently hydrogen, deuterium, a cyano group, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, and
at least one of R¹ to R⁴ is a substituted amine group represented by Chemical Formula a, 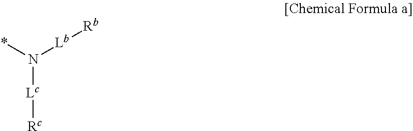
wherein, in Chemical Formula a,
L^{b} and L^{c} are each independently a single bond, a substituted or unsubstituted C6 to C20 arylene group, a substituted or unsubstituted C2 to C20 heterocyclic group, or a combination thereof,
R^{b} and R^{c} are each independently a substituted or unsubstituted C6 to C30 aryl group, a substituted or unsubstituted C2 to C30 heterocyclic group, or a combination thereof, and
* is a linking point with L¹, L², L³, or L⁴; 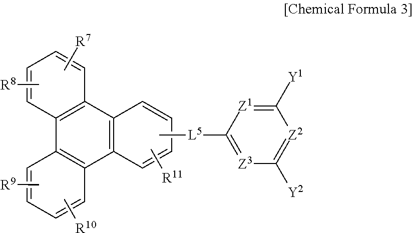
wherein, in Chemical Formula 3,
Z¹ to Z³ are each independently N or CR^{d},
at least two of Z¹ to Z³ are N,
Y¹ and Y² are each independently hydrogen, deuterium, a substituted or unsubstituted C1 to C30 alkyl group, a substituted or unsubstituted C6 to C30 aryl group, a substituted or unsubstituted C2 to C30 heterocyclic group, a halogen, a cyano group, or a combination thereof,
L⁵ is a single bond or a substituted or unsubstituted C6 to C20 arylene group, and
R⁷ to R¹¹ and R^{d} are each independently hydrogen, deuterium, a substituted or unsubstituted C1 to C30 alkyl group, a substituted or unsubstituted C6 to C30 aryl group, a halogen, a cyano group, or a combination thereof.

2. The composition as claimed in claim 1, wherein the first compound is represented by one of Chemical Formula 1A to Chemical Formula 1F: 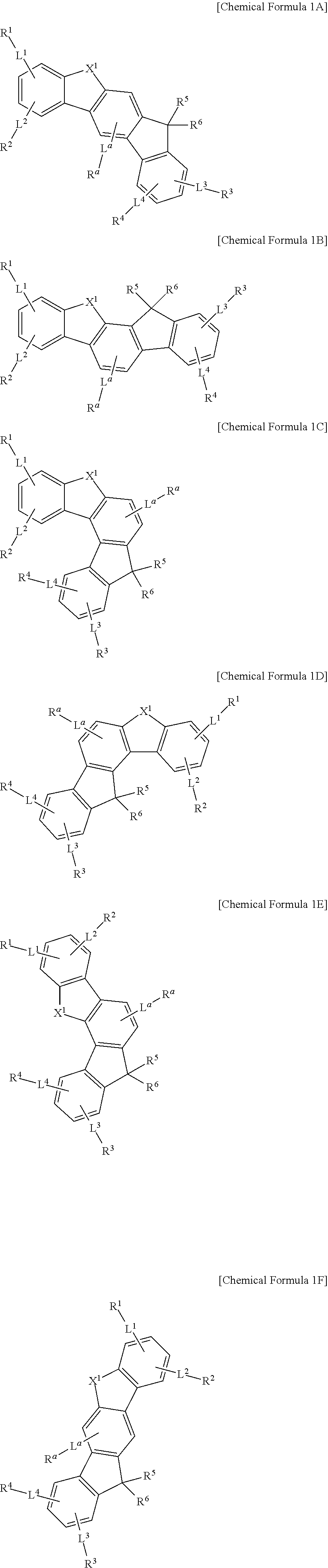
wherein, in Chemical Formula 1A to Chemical Formula 1F,
X¹ is O or S,
L^{a} and L¹ to L⁴ are each independently a single bond, a substituted or unsubstituted C6 to C20 arylene group, a substituted or unsubstituted C2 to C20 heterocyclic group, or a combination thereof,
R^{a} and R¹ to R⁶ are each independently hydrogen, deuterium, a cyano group, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, and
at least one of R¹ to R⁴ is a substituted amine group represented by Chemical Formula a, 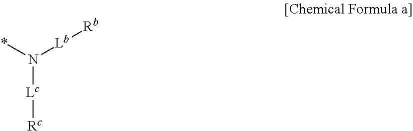
wherein, in Chemical Formula a,
L^{b} and L^{c} are each independently a single bond, a substituted or unsubstituted C6 to C20 arylene group, a substituted or unsubstituted C2 to C20 heterocyclic group, or a combination thereof,
R^{b} and R^{c} are each independently a substituted or unsubstituted C6 to C30 aryl group, a substituted or unsubstituted C2 to C30 heterocyclic group, or a combination thereof, and
* is a linking point with L¹, L², L³, or L⁴.

3. The composition as claimed in claim 1, wherein the first compound is represented by Chemical Formula 1E-1-1 or Chemical Formula 1E-2-2: 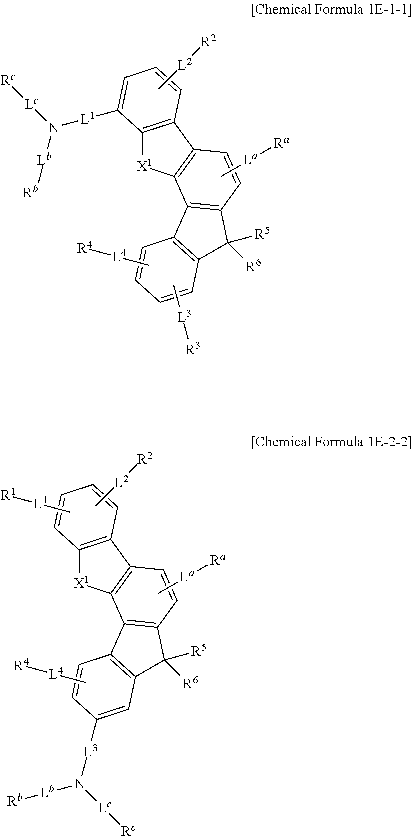
wherein, in Chemical Formula 1E-1-1 and Chemical Formula 1E-2-2,
X¹ is O or S,
L^{a} and L¹ to L⁴ are each independently a single bond, a substituted or unsubstituted C6 to C20 arylene group, a substituted or unsubstituted C2 to C20 heterocyclic group, or a combination thereof,
R^{a} and R¹ to R⁶ are each independently hydrogen, deuterium, a cyano group, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof,
L^{b} and L^{c} are each independently a single bond, a substituted or unsubstituted C6 to C20 arylene group, a substituted or unsubstituted C2 to C20 heterocyclic group, or a combination thereof, and
R^{b} and R^{c} are each independently a substituted or unsubstituted phenyl group, a substituted or unsubstituted biphenyl group, a substituted or unsubstituted terphenyl group, a substituted or unsubstituted anthracenyl group, a substituted or unsubstituted naphthyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted dibenzofuranyl group, a substituted or unsubstituted dibenzothiophenyl group, or a monovalent fused ring group of a compound represented by a combination of Chemical Formula 1 and Chemical Formula 2.

4. The composition as claimed in claim 1, wherein the second compound is represented by Chemical Formula 3A or Chemical Formula 3B: 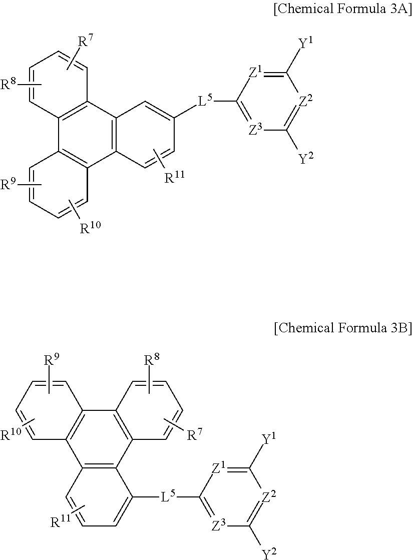
wherein, in Chemical Formulae 3A and 3B,
Z¹ to Z³ are each independently N or CR^{d},
at least two of Z¹ to Z³ are N,
Y¹ and Y² are each independently hydrogen, deuterium, a substituted or unsubstituted C1 to C30 alkyl group, a substituted or unsubstituted C6 to C30 aryl group, a substituted or unsubstituted C2 to C30 heterocyclic group, a halogen, a cyano group, or a combination thereof,
L⁵ is a single bond or a substituted or unsubstituted C6 to C20 arylene group, and
R⁷ to R¹¹ and R^{d} are each independently hydrogen, deuterium, a substituted or unsubstituted C1 to C30 alkyl group, a substituted or unsubstituted C6 to C30 aryl group, a halogen, a cyano group, or a combination thereof.

5. The composition as claimed in claim 1, wherein Y¹ and Y² are each independently a substituted or unsubstituted phenyl group, a substituted or unsubstituted biphenyl group, a substituted or unsubstituted terphenyl group, a substituted or unsubstituted naphthyl group, a substituted or unsubstituted triphenylene group, a substituted or unsubstituted pyridinyl group, a substituted or unsubstituted pyrimidinyl group, a substituted or unsubstituted triazinyl group, a substituted or unsubstituted dibenzofuranyl group, a substituted or unsubstituted dibenzothiophenyl group, or a combination thereof.

6. The composition as claimed in claim 1, wherein L⁵ is a single bond, a substituted or unsubstituted phenylene group, a substituted or unsubstituted biphenylene group, or a substituted or unsubstituted terphenylene group.

7. The composition as claimed in claim 1, wherein L⁵ is a single bond, an unsubstituted phenylene group, an unsubstituted biphenylene group, an unsubstituted terphenylene group, a phenylene group substituted with a phenyl group or a cyano group, a biphenylene group substituted with a phenyl group or a cyano group, or a terphenylene group substituted with a phenyl group or a cyano group.

8. The composition as claimed in claim 7, wherein L⁵ is a single bond or a linking group of the following Group I,
[Group I] 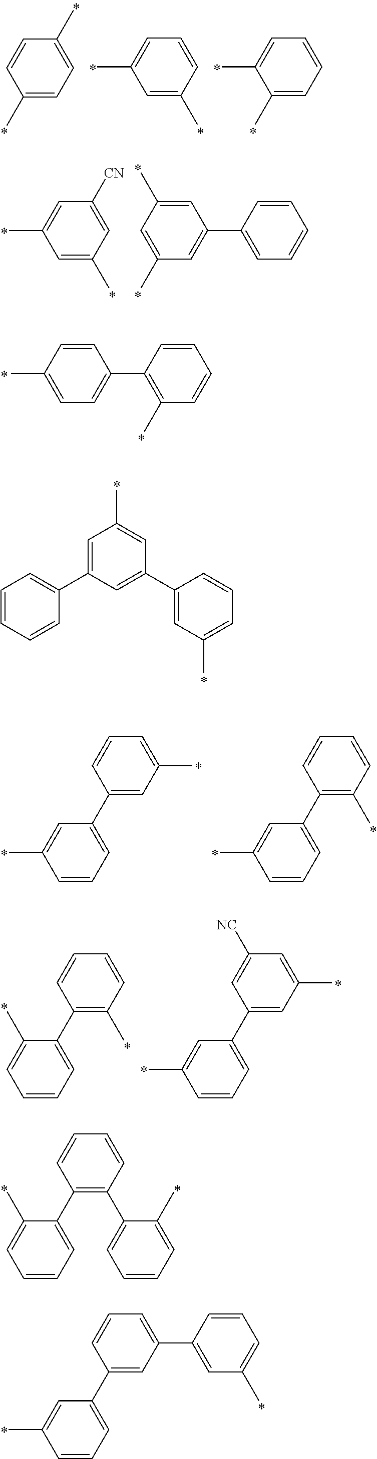
wherein, in Group I, each * is a linking point.

9. The composition as claimed in claim 1, wherein the moiety 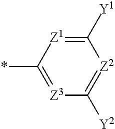
of Chemical Formula 3 is a moiety of the following Group II,
[Group II] 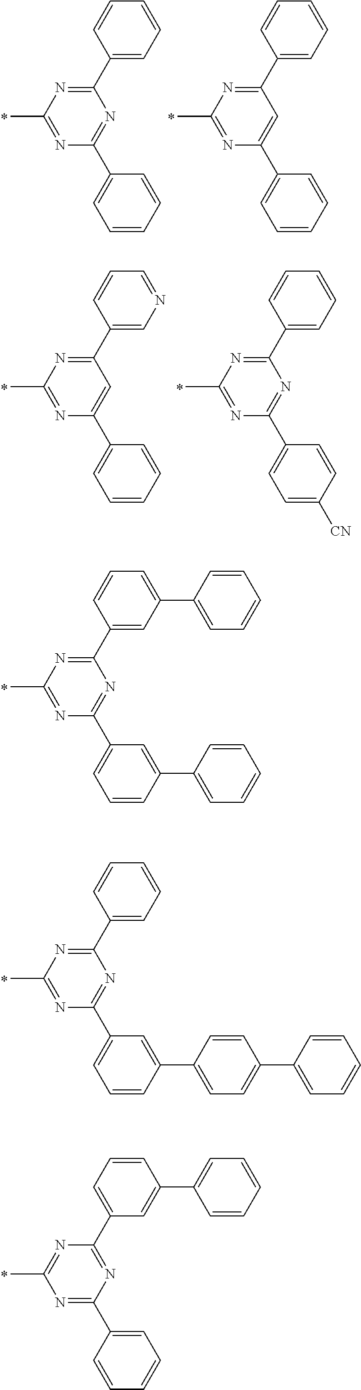 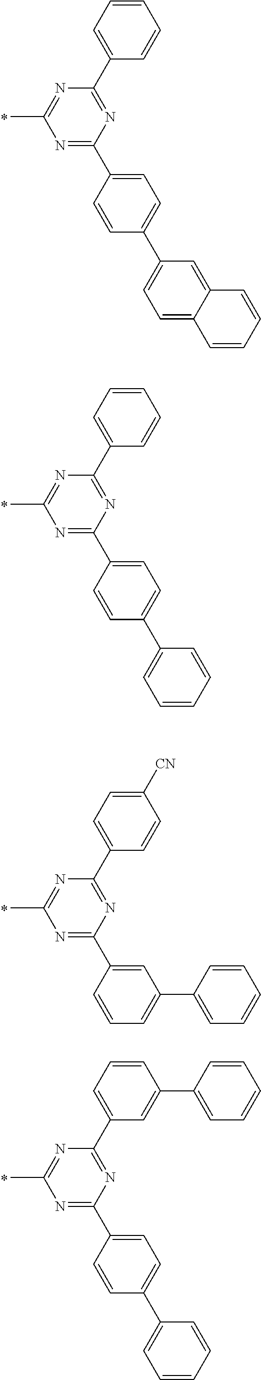 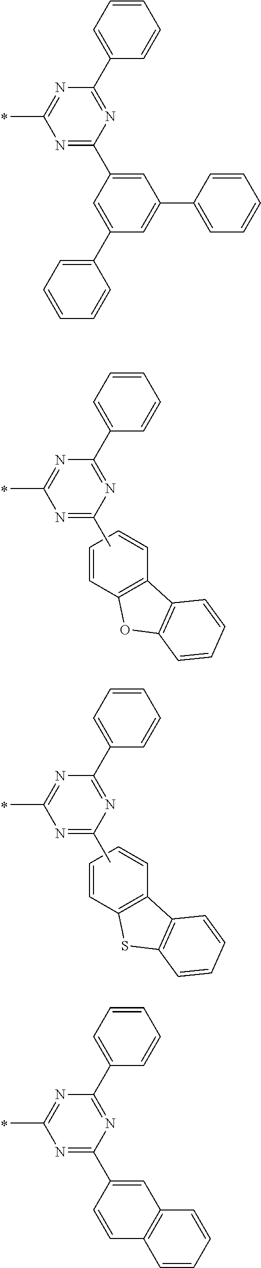
wherein, in Group II, * is a linking point.

10. The composition as claimed in claim 1, wherein:
the first compound is represented by Chemical Formula 1E-2-2, and
the second compound is represented by Chemical Formula 3A-1: 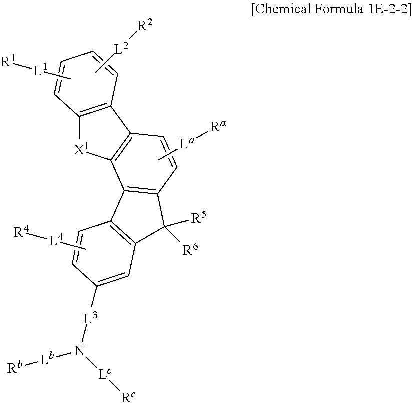
wherein, in Chemical Formula 1E-2-2,
X¹ is O or S,
L^{a}, L^{b}, L^{c}, and L¹ to L⁴ are each independently a single bond, a substituted or unsubstituted phenylene group, a substituted or unsubstituted biphenylene group, a substituted or unsubstituted terphenylene group, or a substituted or unsubstituted naphthylene group,
R^{a}, R¹, R², and R⁴ are each independently hydrogen, deuterium, a cyano group, a substituted or unsubstituted C1 to C30 alkyl group, a substituted or unsubstituted C6 to C30 aryl group, a substituted or unsubstituted C2 to C30 heterocyclic group, or a combination thereof,
R⁵ and R⁶ are each independently a substituted or unsubstituted C1 to C10 alkyl group or a substituted or unsubstituted C6 to C20 aryl group, and
R^{b} and R^{c} are each independently a substituted or unsubstituted phenyl group, a substituted or unsubstituted biphenyl group, a substituted or unsubstituted anthracenyl group, a substituted or unsubstituted naphthyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted dibenzofuranyl group, a substituted or unsubstituted dibenzothiophenyl group, or a monovalent fused ring group of a compound represented by a combination of Chemical Formula 1 and Chemical Formula 2; 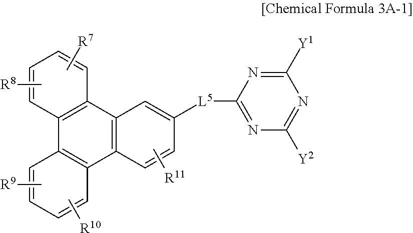
wherein, in Chemical Formula 3A-1,
Y¹ and Y² are each independently hydrogen, deuterium, a substituted or unsubstituted C1 to C30 alkyl group, a substituted or unsubstituted C6 to C30 aryl group, a substituted or unsubstituted C2 to C30 heterocyclic group, a halogen, a cyano group, or a combination thereof,
L⁵ is a single bond or a substituted or unsubstituted C6 to C20 arylene group, and
R⁷ to R¹¹ are each independently hydrogen, deuterium, a substituted or unsubstituted C1 to C30 alkyl group, a substituted or unsubstituted C6 to C30 aryl group, a halogen, a cyano group, or a combination thereof.

11. The composition as claimed in claim 1, further comprising a dopant.

12. An organic optoelectronic device, comprising:
an anode and a cathode facing each other,
at least one organic layer between the anode and the cathode,
wherein the organic layer includes the composition for an organic optoelectronic device as claimed in claim 1.

13. The organic optoelectronic device as claimed in claim 12, wherein:
the organic layer includes a light emitting layer, and
the light emitting layer includes the composition.

14. The organic optoelectronic device as claimed in claim 13, wherein the first compound and the second compound are phosphorescent hosts of the light emitting layer.

15. The organic optoelectronic device as claimed in claim 12, wherein the composition is a red light emitting composition.

16. A display device comprising the organic optoelectronic device as claimed in claim 12.
